(19) United States
(12) Patent Application Publication
(10) Pub. No.: US 2003/0180891 A1
(43) Pub. Date: Sep. 25, 2003

(54) Connective tissue growth factor-4

(75) Inventors: Paul E. Young, Gaithersburg, MD (US); Steven M. Ruben, Brookeville, MD (US)

Correspondence Address: HUMAN GENOME SCIENCES INC, 9410 KEY WEST AVENUE, ROCKVILLE, MD, 20850

(73) Assignee: Human Genome Sciences, Inc., Rockville, MD

(21) Appl. No.: 10/394,015

(22) Filed: Mar. 24, 2003

Related U.S. Application Data
(63) Continuation of application No. 09/325,019, filed on Jun. 3, 1999.
(60) Provisional application No. 60/088,320, filed on Jun. 5, 1998.

(51) Int. Cl. C12P 21/02; C07K 14/475; C12N 5/06; C07H 21/04

(52) U.S. Cl. 435/69.4; 435/320.1; 435/325; 530/399; 536/23.5

(57) ABSTRACT

The present invention relates to a novel human protein called Connective Tissue Growth Factor-4, and isolated polynucleotides encoding this protein. Also provided are vectors, host cells, antibodies, and recombinant methods for producing this human protein. The invention further relates to diagnostic and therapeutic methods useful for diagnosing and treating disorders related to this novel human protein.

This application is a continuation of U.S. application Ser. No. 09/325,019, filed on Jun. 3, 1999, which claims benefit under 35 U.S.C. §119(e) of U.S. Provisional Application No. 60/088,320, filed on Jun. 5, 1998. Each of the above referenced applications is hereby incorporated by reference in its entirety.

### FIELD OF THE INVENTION

The present invention relates to a novel human gene encoding a polypeptide which is a member of the CCN (connective tissue growth factor [CTGF], Cyr61/Cef10, neuroblastoma overexpressed gene [Nov]) family of proteins (which consists of secreted cysteine-rich proteins with growth regulatory functions). More specifically, the present invention relates to a polynucleotide encoding a novel human polypeptide named Connective Tissue Growth Factor-4, or “CTGF-4”. This invention also relates to CTGF-4 polypeptides, as well as vectors, host cells, antibodies directed to CTGF-4 polypeptides, and the recombinant methods for producing the same. Also provided are diagnostic methods for detecting disorders related to connective tissues (for example, cancer, arthritis, fibrosis, atherosclerosis, and osteoporosis), and therapeutic methods for treating such disorders. The invention further relates to screening methods for identifying agonists and antagonists of CTGF-4 activity.

### BACKGROUND OF THE INVENTION

Growth factors are a class of secreted cysteine-rich polypeptides that stimulate target cells to proliferate, differentiate, and organize in developing and mature tissues. The action of growth factors is dependent on their binding to specific receptors, which stimulate a signaling event within the cell. Examples of some well-studied growth factors include platelet-derived growth factor (PDGF), insulin-like growth factor (IGF-I), transforming growth factor (TGF)-α, epidermal growth factor (EGF), and fibroblast growth factor (FGF). This group of growth factors is important for normal growth, differentiation, morphogenesis of the cartilaginous skeleton of an embryo, and cell growth. Among some of the functions that have been reported for these growth factors are wound healing, tissue repair and regeneration, implant fixation, and stimulation of an increase in bone mass.

PDGF is a cationic, heat-stable protein found in the alpha-granules of circulating platelets and is known to be a mitogen and chemotactic agent for connective tissue cells such as fibroblasts and smooth muscle cells. Because of the activities of this molecule, PDGF is believed to be a major factor involved in the normal healing of wounds and pathologically contributes to such diseases as atherosclerosis and fibrotic diseases. PDGF is a dimeric molecule consisting of an A chain and a B chain. The chains form heterodimers or homodimers and all combinations isolated to date are biologically active.

Studies on the role of various growth factors in tissue regeneration and repair have led to the discovery of PDGF-like proteins. These proteins share both immunological and biological activities with PDGF and can be blocked with antibodies specific to PDGF.

U.S. Pat. No. 5,408,040 issued to Grotendorst, et al. (1995) discloses a PDGF-like protein called connective tissue growth factor (CTGF). CTGF reportedly plays a significant role in the normal development, growth, and repair of human tissue. Isolation of the CTGF protein and cloning of the corresponding cDNA was a significant discovery since CTGF was a previously unknown growth factor having mitogenic and chemotactic activities for connective tissue cells. Although the biological activity of CTGF is similar to that of PDGF, CTGF is the product of a gene unrelated to the A or B chain genes of PDGF.

Since CTGF is produced by endothelial and fibroblastic cells, both of which are present at the site of a wound, it is probable that CTGF functions as a growth factor in wound healing. Accordingly, it is believed that the CTGF polypeptide could be used as a therapeutic in cases in which there is impaired healing of skin wounds or where there is a need to augment the normal healing process.

Pathologically, CTGF may also be involved in diseases in which there is an overgrowth of connective tissue cells or an enhanced production of extracellular matrix components. Such diseases include cancers, fibrosis, and atherosclerosis. For example, CTGF gene expression is elevated in the skin of patients with systemic sclerosis (Igarashi, et al., J. Invest. Den atol. 105:280-284 (1995)). In addition, CTGF is also expressed in several fibrotic skin diseases, such as localized scleroderma, keloid scars, nodular fasciitis, and eosinophilic fascuitis, suggesting a pathogenic role for this molecule in skin fibrosis (Igarashi, et al., J. Invest. Dermatol. 106:729-733 (1996)). Oemar and colleagues (Circulation 92(8) Supplement 1, Abstract 0811 (October 1995)) report that human CTGF is expressed at 5-10 fold higher levels in the aorta. When compared to internal mammary arteries, the aorta is highly prone to develop atherosclerosis. Thus, Oemar and coworkers (supra) hypothesize that human CTGF plays an essential role in the development and progression of atherosclerosis. Therapeutically, CTGF antibodies or fragments thereof can neutralize the biological activity of CTGF in diseases where CTGF is inducing the overgrowth of tissue (Grotendorst, et al., supra). Additionally, antibodies to CTGF polypeptide or fragments thereof may be valuable diagnostic tools.

Thus, there is a need for polypeptides that can be used in the development of diagnostics and therapeutics for various connective tissue related disorders. Such factors may be involved in the development, progression and repair of human tissues, as well as in the development and progression of various connective tissue related disorders. Therefore, there is a need for identification and characterization of such human polypeptides which can play a role in detecting, preventing, ameliorating or correcting the above mentioned and other disorders.

### SUMMARY OF THE INVENTION

The present invention relates to a novel polynucleotide and the encoded polypeptide of CTGF-4. Moreover, the present invention relates to vectors, host cells, antibodies, and recombinant methods for producing the polypeptides and polynucleotides. Also provided are diagnostic methods for detecting disorders relates to the polypeptides, and therapeutic methods for treating such disorders. The invention further relates to screening methods for identifying binding partners of CTGF-4.

### BRIEF DESCRIPTION OF THE DRAWINGS

FIGS. 1A, 1B, 1C, and 1D show the nucleotide sequence (SEQ ID NO:1) and the deduced amino acid sequence (SEQ ID NO:2) of CTGF-4. Among other potentially less apparent regions of sequence identity and homology, CTGF-4 contains eleven polypeptide domains which are comprised of amino acid sequences which are highly conserved between CTGF-4 and other CCN family members. The eleven CCN family member Conserved Domains are double-underlined and labeled as “CD-I” through “CD-XI” in FIGS. 1A, 1B, 1C, and 1D. Four asparagine residues in the CTGF-4 polypeptide sequence conform to an accepted consensus sequence which indicates the potential for N-linked glycosylation (the consensus sequence is N-X-S or N-X-T, where N=asparagine, X=any amino acid residue, S=serine, and T=threonine). The potentially N-linked asparagine residues are presented in the sequence shown in FIGS. 1A, 1B, 1C, and 1D in boldface type (N) and are marked with a boldface pound sign (#) above the nucleotide sequence encoding the asparagine residue.

FIGS. 2A, 2B, 2C, 2D, and 2E show the regions of identity between the amino acid sequences of CTGF-4 protein and four CCN family members as determined by MegAlign analysis. In addition to CTGF-4, the CCN growth factor family members shown in FIGS. 2A, 2B, 2C, 2D, and 2E are mouse ELM-1 protein (ATCC Accession No.: AB004873; SEQ ID NO:3), human CTGF protein (ATCC Accession Nos.: M92934, M36965, and S56201; SEQ ID NO:4), human Cyr61 protein (ATCC Accession No.: U62015; SEQ ID NO:5), and human NOV protein (ATCC Accession No.: X96584; SEQ ID NO:6). In positions within the alignment where at least two proteins have an identical residue, the amino acid residues at that position are shaded. By examining the shaded regions of amino acid sequence, the skilled artisan can readily identify conserved domains between the five polypeptides.

FIG. 3 shows an analysis of the CTGF-4 amino acid sequence. Alpha, beta, turn and coil regions; hydrophilicity and hydrophobicity; amphipathic regions; flexible regions; antigenic index and surface probability are shown. In the “Antigenic Index or Jameson-Wolf” graph, the positive peaks indicate locations of the highly antigenic regions of the CTGF-4 protein, i.e., regions from which epitope-bearing peptides of the invention can be obtained. The domains defined by these graphs are contemplated by the present invention.

FIG. 4 shows an RNA blot hybridization (Northern blot) analyzing the expression pattern of CTGF-4 in a number of cell and tissue types. Markers on the blot include (from top to bottom; position indicated by a small horizontal bar on the right-hand side of the gel) 9.5 kb, 7.5 kb, 4.4 kb, 2.4 kb, and 1.35 kb. Tissues analyzed on the gel include (from left to right; each sample lane is indicated by a dot at the top of the lane) pancreas, kidney, smooth muscle, lung, liver, placenta, brain, and heart.

### DETAILED DESCRIPTION

Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. However, a nucleic acid contained in a clone that is a member of a library (e.g., a genomic or cDNA library) that has not been isolated from other members of the library (e.g., in the form of a homogeneous solution containing the clone and other members of the library) or which is contained on a chromosome preparation (e.g., a chromosome spread), is not “isolated” for the purposes of this invention.

In the present invention, a “secreted” CTGF-4 protein refers to a protein capable of being directed to the ER, secretory vesicles, or the extracellular space as a result of a signal sequence, as well as a CTGF-4 protein released into the extracellular space without necessarily containing a signal sequence. If the CTGF-4 secreted protein is released into the extracellular space, the CTGF-4 secreted protein can undergo extracellular processing to produce a “mature” CTGF-4 protein. Release into the extracellular space can occur by many mechanisms, including exocytosis and proteolytic cleavage.

Although a “mature” CTGF-4 protein refers to a CTGF-4 polypeptide lacking a secretory signal peptide, a CTGF-4 protein may be further biologically processed to a mature form which lacks additional N- or C-terminal or central or a combination of N- or C-terminal or central amino acid residues. Such a “biologically mature” form of CTGF-4 may consist of a biologically processed monomer, homodimer, heterodimer, trimer (composed of three identical subunits, two identical and one unique subunits or three unique subunits) or a polymer consisting of four or more subunits (such a polymer may consist of any combination of identical or unique subunits). Moreover, any subunit of any biologically mature form of CTGF-4 may associate in a parallel or in an anti-parallel conformation with regard to any other biologically mature CTGF-4 subunit.

It is well-known in the art that many secreted proteins are secreted from the cell as a mature form which may have a highly reduced, a slightly reduced or an equal amount of a particular biological activity when compared to a further processed biologically mature form. In the case of CTGF-4 of the present invention, CTGF-4 may be secreted as a mature form which may have a reduced level of a particular biological activity when compared to a biologically mature form of CTGF-4, while at the same time having the same level of a second particular biological activity. Further, CTGF-4 may be secreted as a mature form which may have an identical or nearly identical particular biological activity when compared to a biologically mature form of CTGF-4, in which case, although further processing of mature CTGF-4 may occur in vivo or in vitro or both, it does not substantially affect the particular biological activity. It is routine in the art to empirically determine the relationship between biological processing of CTGF-4 and the level of a particular biological activity.

As used herein, a CTGF-4 “polynucleotide” refers to a molecule having a nucleic acid sequence contained in SEQ ID NO:1 or the cDNA contained within the clone deposited with the ATCC. For example, the CTGF-4 polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, a CTGF-4 “polypeptide” refers to a molecule having the translated amino acid sequence generated from the polynucleotide as broadly defined.

In the present invention, the full length CTGF-4 sequence identified as SEQ ID NO:1 was generated by overlapping sequences contained in multiple clones (a process termed “contig analysis”). Two representative clones containing all or most of the sequence for SEQ ID NO:1 were deposited with the American Type Culture Collection (“ATCC”) on Apr. 29, 1998, and the deposit was given the ATCC Deposit Number 209816. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure. The deposit contains an equal amount of two independent cDNA clones encoding CTGF-4. The clones are designated HWHGU74 and HWHGU74S15. The cDNA clone designated HWHGU74S15 contains an 5′ fragment of the CTGF-4 open reading frame which overlaps with the 5′ end of the HWHGU74 cDNA clone and extends the known CTGF-4 sequence approximately 700 nucleotides in the 5′ direction. It would be routine for one of skill in the art to use the two clones in the deposit (for example, by using an overlapping PCR approach) to generate a single cDNA clone which contains all of the nucleotide sequence shown as SEQ ID NO:1.

A CTGF-4 “polynucleotide” also includes those polynucleotides capable of hybridizing, under stringent hybridization conditions, to sequences contained in SEQ ID NO:1, the complement thereof, or the cDNA within the deposited clone. “Stringent hybridization conditions” refers to an overnight incubation at 42° C. in a solution comprising 50% formamide, 5×SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

Also contemplated are nucleic acid molecules that hybridize to the CTGF-4 polynucleotides at lower stringency hybridization conditions. Changes in the stringency of hybridization and signal detection are primarily accomplished through the manipulation of formamide concentration (lower percentages of formamide result in lowered stringency), salt conditions, or temperature. For example, lower stringency conditions include an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide”, since such a polynucleotide would hybridize to any nucleic acid molecule containing a poly (A) stretch or the complement thereof (e.g., practically any double-stranded cDNA clone).

The CTGF-4 polynucleotide can be composed of any polyribonucleotide or polydeoxribonucleotide, which may be unmodified RNA or DNA or modified RNA or DNA. For example, CTGF-4 polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the CTGF-4 polynucleotides can be composed of triple-stranded regions comprising RNA or DNA or both RNA and DNA. CTGF-4 polynucleotides may also contain one or more modified bases or DNA or RNA backbones modified for stability or for other reasons. “Modified” bases include, for example, tritylated bases and unusual bases such as inosine. A variety of modifications can be made to DNA and RNA, thus, “polynucleotide” embraces chemically, enzymatically, or metabolically modified forms.

CTGF-4 polypeptides can be composed of amino acids joined to each other by peptide bonds or modified peptide bonds, i.e., peptide isosteres, and may contain amino acids other than the 20 gene-encoded amino acids. The CTGF-4 polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the CTGF-4 polypeptide, including the peptide backbone, the amino acid side-chains and the amino or carboxyl termini. It will be appreciated that the same type of modification may be present in the same or varying degrees at several sites in a given CTGF-4 polypeptide. Also, a given CTGF-4 polypeptide may contain many types of modifications. CTGF-4 polypeptides may be branched, for example, as a result of ubiquitination, and they may be cyclic, with or without branching. Cyclic, branched, and branched cyclic CTGF-4 polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, lodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pp. 1-12 (1983); Seifter, et al., Meth. Enzymol. 182:626-646 (1990); Rattan, et al., Ann. NY Acad. Sci. 663:48-62 (1992)).

“SEQ ID NO:1” refers to a CTGF-4 polynucleotide sequence while “SEQ ID NO:2” refers to a CTGF-4 polypeptide sequence.

A CTGF-4 polypeptide “having biological activity” refers to polypeptides exhibiting activity similar, but not necessarily identical to, an activity of a CTGF-4 polypeptide, including mature forms, as measured in a particular biological assay, with or without dose dependency. In the case where dose dependency does exist, it need not be identical to that of the CTGF-4 polypeptide, but rather substantially similar to the dose-dependence in a given activity as compared to the CTGF-4 polypeptide (i.e., the candidate polypeptide will exhibit greater activity or not more than about 25-fold less and, preferably, not more than about tenfold less activity, and most preferably, not more than about three-fold less activity relative to the CTGF-4 polypeptide).

CTGF-4 Polynucleotides and Polypeptides

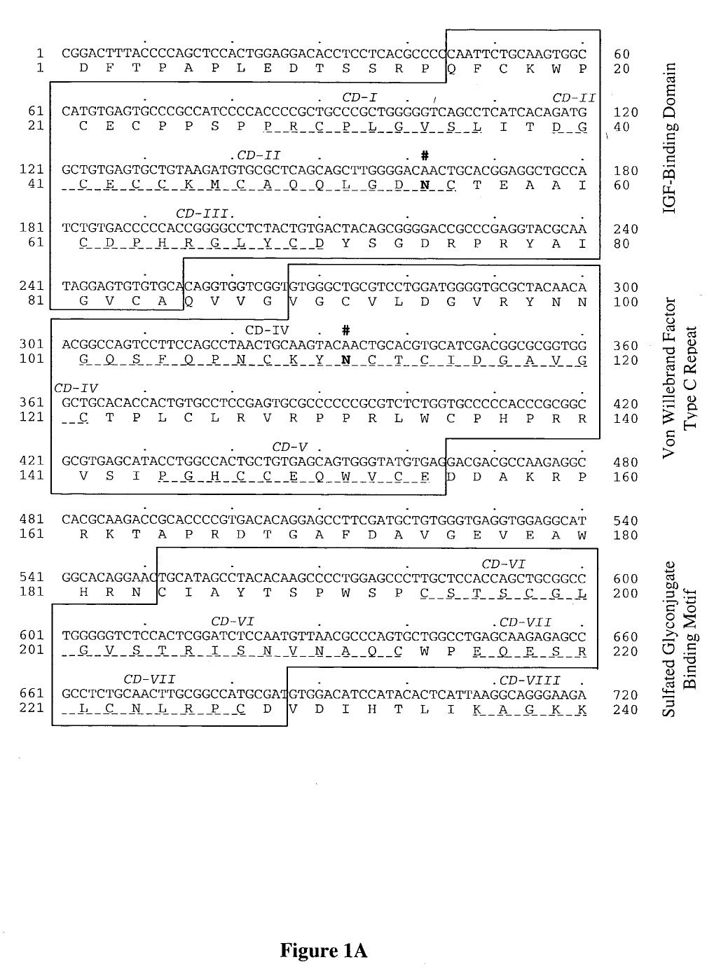

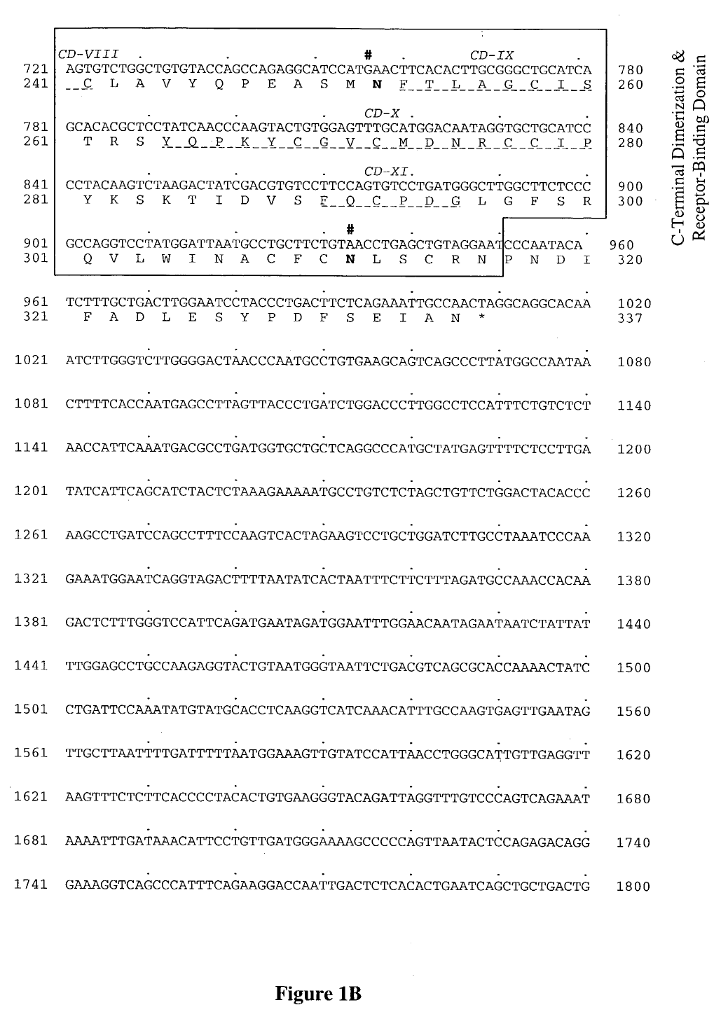

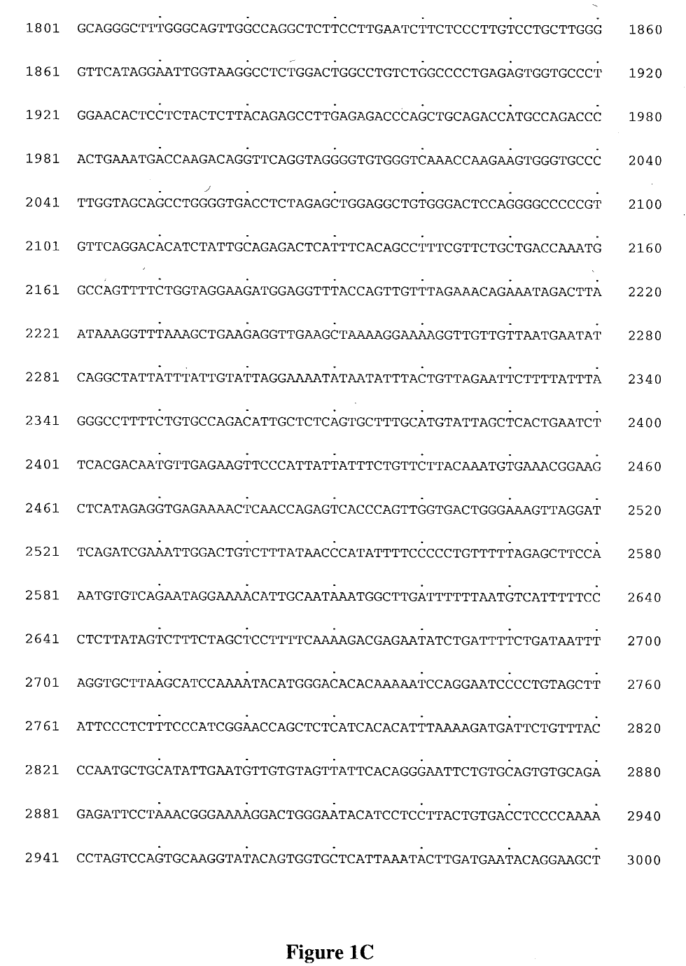

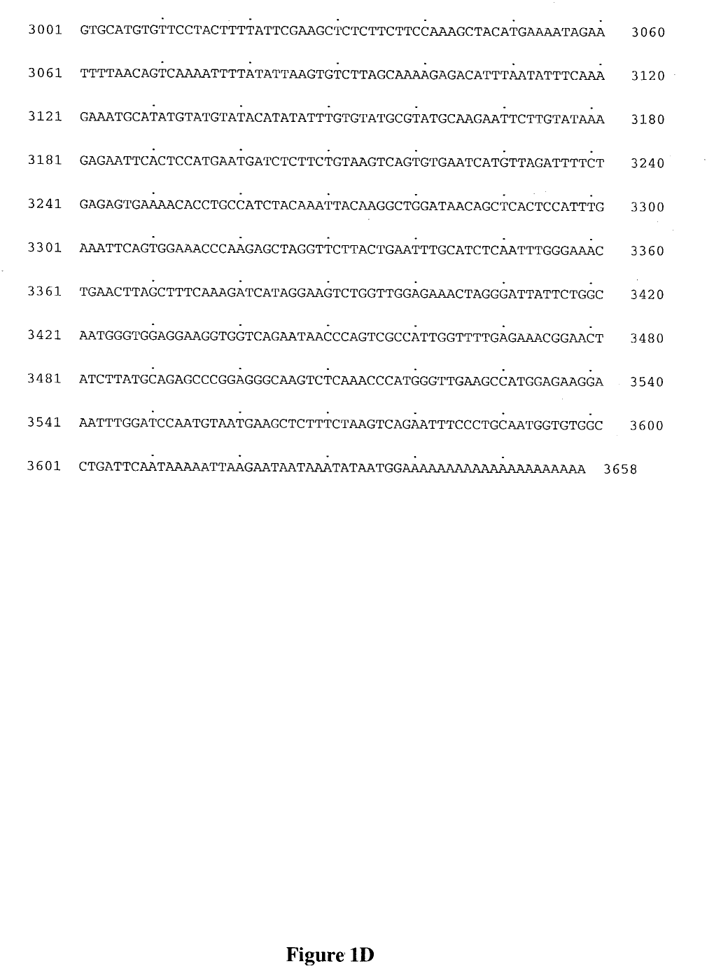

Clone HWHGU74 was isolated from a serum-treated smooth muscle cDNA library. This clone contains the entire coding region identified as SEQ ID NO:2. The deposited clone contains a cDNA having a total of 3,658 nucleotides, which encodes 335 amino acid residues of a predicted open reading frame. (See FIGS. 1A, 1B, 1C, and 1D.) The open reading frame begins in frame at a N-terminal aspartic acid residue located at nucleotide position 3, and ends at a stop codon at nucleotide position 1011. The predicted molecular weight of the CTGF-4 protein is approximately 37 kDa.

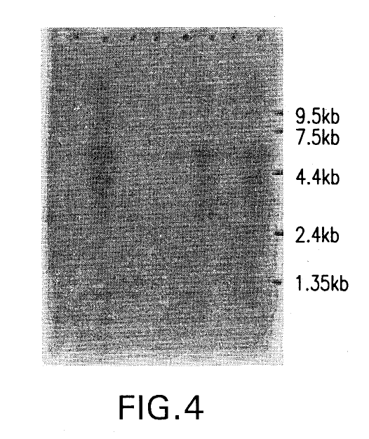

Subsequent Northern analysis also showed high levels of CTGF-4 expression in fetal liver, lymph node, kidney, and ovary, and lower levels of expression in spleen, bone marrow, heart, placenta, lung, liver, and prostate. Such an expression pattern is consistent with the Northern blot shown as FIG. 4.

The Northern blot shown as FIG. 4 provides evidence that in the case where the insert of the cDNA clone designated HWHGU74 is used as a labeled probe in an RNA blot (i.e. a Northern blot) hybridization analysis, a truly full-length CTGF-4 molecule may be isolated. The blot shown in FIG. 4 shows hybridization of the CTGF-4 probe to three species. The predominant species has a mobility of 5.5-6 kb (approximately 5.75 kb) and the two lesser species have mobilities of 4-4.4 and 2.8-3.5 kb (approximately 4.2 and 3.15 kb, respectively). It is believed that each of the three species encodes the full-length CTGF-4 open reading frame and differs only in the site of polyadenylation. Three different polyadenylation sites have been identified in the 3′ untranslated region of the CTGF-4 nucleotide sequence shown as SEQ ID NO: 1 and differential usage of the three polyadenylation sites may result in the three different species of transcripts detected in FIG. 4. Although the three species differ in size, it is believed that each contains the complete CTGF-4 open reading frame. Furthermore, since the blot in FIG. 4 identifies three CTGF-4 species, it is appreciated that when the insert of the cDNA clone designated HWHGU74 is used as a labeled probe in an RNA blot (i.e. a Northern blot) hybridization analysis, each of the three potential splice variants of the CTGF-4 molecule may be isolated.

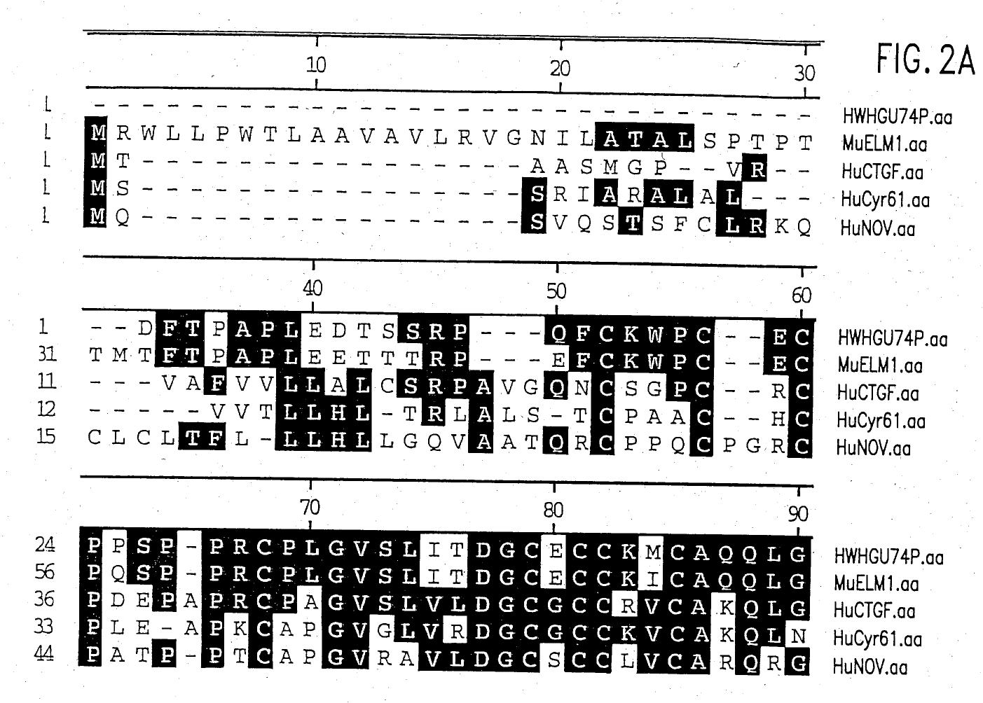

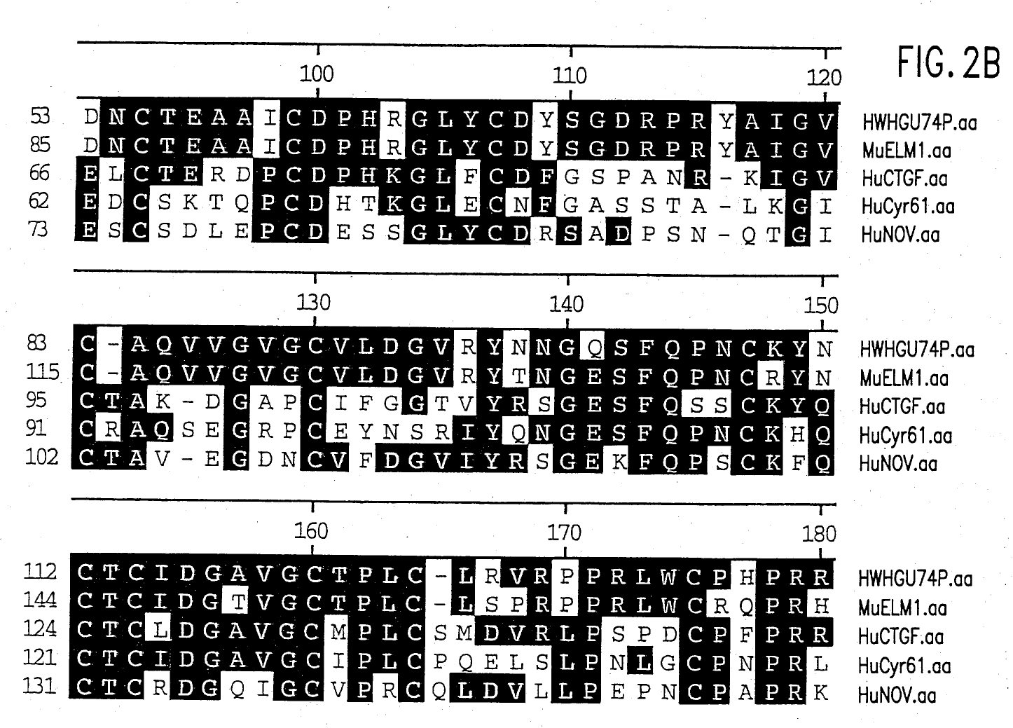

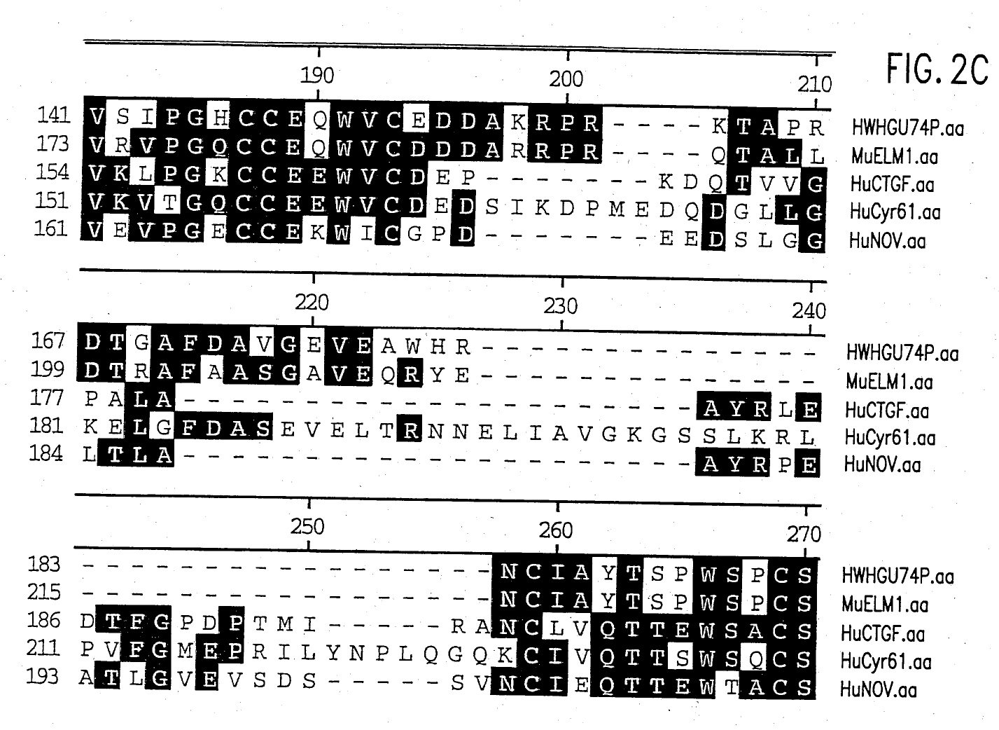

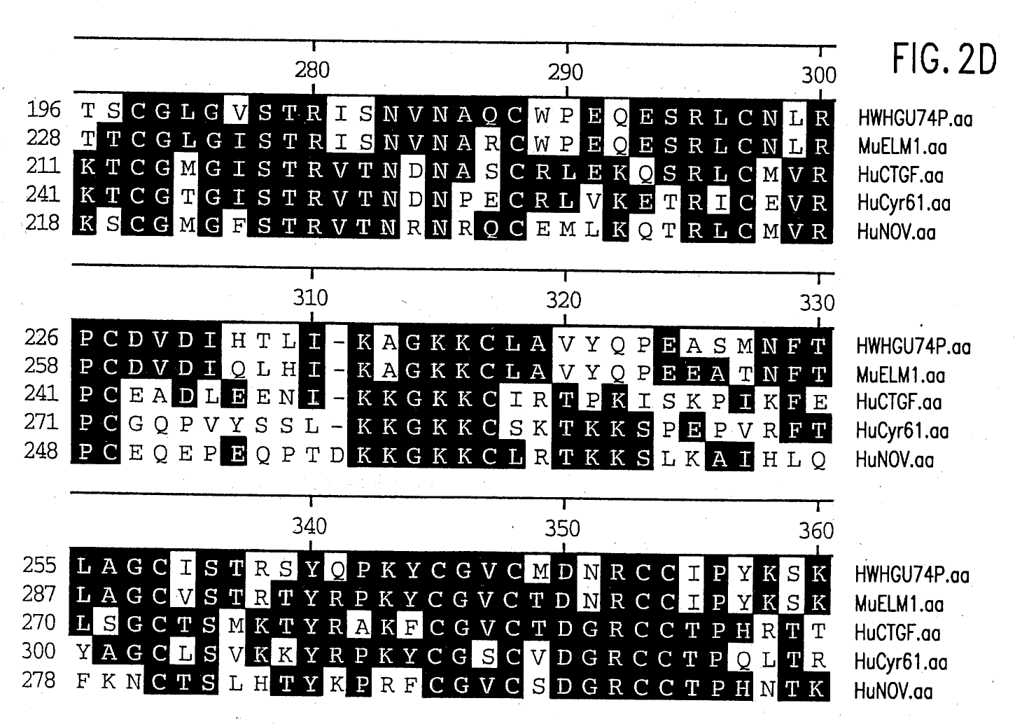

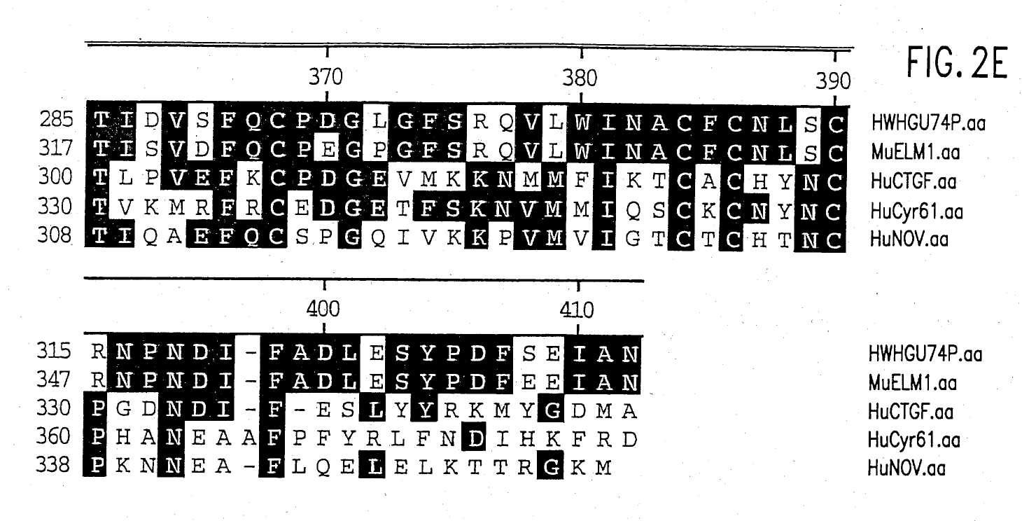

Using BLAST analysis, SEQ ID NO:2 was found to be homologous to members of the CCN family of growth factors. Particularly, SEQ ID NO:2 contains domains homologous to the translation product of the mouse mRNA for ELM-1 (SEQ ID NO:3) and to human CTGF (SEQ ID NO:4), Cyr6l (SEQ ID NO:5), and NOV (SEQ ID NO:6), including the following highly conserved domains: (a) an IGF-binding homology domain located at about amino acids 15-84; (b) a von Willebrand factor type C repeat located at about amino acids 89-154; (c) a sulfated glycoconjugate-binding motif located at about amino acids 184-228; (d) a C-terminal dimerization and receptor-binding domain located at about amino acids 241-316; (e) a predicted Conserved Domain I (CD-I) domain located at about amino acids 28-36; (f) a predicted Conserved Domain II (CD-II) domain located at about amino acids 39-55; (g) a predicted Conserved Domain III (CD-III) domain located at about amino acids 61-70; (h) a predicted Conserved Domain IV (CD-IV) domain located at about amino acids 101-121; (i) a predicted Conserved Domain V (CD-V) domain located at about amino acids 144-154; (O) a predicted Conserved Domain VI (CD-VI) domain located at about amino acids 194-213; (k) a predicted Conserved Domain VII (CD-VII) domain located at about amino acids 216-227; (1) a predicted Conserved Domain VII (CD-VII) domain located at about amino acids 236-241; (m) a predicted Conserved Domain IX (CD-IX) domain located at about amino acids 253-260; (n) a predicted Conserved Domain X (CD-X) domain located at about amino acids 264-280; and (O) a predicted Conserved Domain XI (CD-XI) domain located at about amino acids 290-295. These polypeptide fragments of CTGF-4 are specifically contemplated in the present invention. Because murine ELM-1 and the other CCN family members shown in FIGS. 2A, 2B, 2C, 2D, and 2E are thought to be important in the regulation of growth of cells comprising connective tissues, the homology between murine ELM-1 and the other CCN family members shown in FIGS. 2A, 2B, 2C, 2D, and 2E and CTGF-4 suggests that CTGF-4 may also be involved in the regulation of growth of cells comprising connective tissues.

Based on the alignment of CTGF-4 with several CCN family members, presented as FIGS. 2A, 2B, 2C, 2D, and 2E, it is likely that the CTGF-4 cDNA clone disclosed in this application is slightly less than a full-length cDNA of a naturally occurring CTGF-4 mRNA. As deduced from FIGS. 2A, 2B, 2C, 2D, and 2E, it is likely that a full-length CTGF-4 cDNA will encode approximately an additional 9, 16, or 32 additional amino acid residues at its N-terminus. Furthermore, since the integers 9, 16, and 32 are approximations based on alignments of CTGF-4 with independent polypeptides, it is also contemplated that a full-length CTGF-4 cDNA is just as likely to encode any integer in the range of 1-50 additional N-terminal amino acid residues as it is to encode 9, 16 or 32 additional residues. For this reason, CTGF-4 cDNAs of the present invention also include those which encode additional N-terminal amino acid residues, particularly amino acid residues which are encoded by a naturally occurring CTGF-4 mRNA. Further, CTGF-4 polypeptides of the present invention include those which possess additional N-terminal amino acid residues, particularly amino acid residues which are encoded by a naturally occurring CTGF-4 mRNA. Moreover, polynucleotides of the present invention also include those which encode additional amino acid residues within the CTGF-4 nucleotide sequence shown as SEQ ID NO:1 of the present invention such that the N-terminus of CTGF-4 polypeptide shown as SEQ ID NO:2 aligns more closely with the N-termini of murine ELM-1, human CTGF, human Cyr61, human NOV or other CCN family members. Likewise, polypeptides of the present invention also include those which possess additional amino acid residues within the CTGF-4 polypeptide sequence shown as SEQ ID NO:2 of the present invention such that the N-terminus of CTGF-4 polypeptide shown as SEQ ID NO:2 aligns more closely with the N-termini of murine ELM-1, human CTGF, human Cyr61, human NOV or other CCN family members.

Moreover, the N-termini of murine ELM-1, human CTGF, human Cyr61, human NOV, and other CCN family members encode a signal peptide which directs secretion of mature forms of the respective molecules from the cell. As a result, it is also highly likely that full-length CTGF-4 molecule of the invention also encodes an N-terminal signal peptide such that the full-length CTGF-4 polypeptide is processed to a mature form and secreted from the cell. The CTGF-4 polynucleotide shown as SEQ ID NO: 1 does not encode a predicted signal peptide, nor does the CTGF-4 polypeptide shown as SEQ ID NO:2 possess an N-terminal signal peptide. However, as mentioned above, since the full-length CTGF-4 polynucleotide likely encodes and the full-length CTGF-4 polypeptide likely possesses a secretory signal peptide, and since it is well-known in the art that secretory signal peptides are largely modular in nature and that they are typically known to direct secretion of any protein to which they are molecularly fused, it is also contemplated herein that the CTGF-4 polypeptides of the present invention may be directed to the cellular secretory pathway by fusion to any one of the secretory signal peptides of murine ELM-1, human CTGF, human Cyr61, human NOV, other CCN family members, any previously described secretory signal peptide or any yet to be described secretory signal peptide.

The CTGF-4 nucleotide sequence identified as SEQ If) NO:1 was assembled from partially homologous (“overlapping”) sequences obtained from the deposited clone, and in some cases, from additional related DNA clones. The overlapping sequences were assembled into a single contiguous sequence of high redundancy (usually three to five overlapping sequences at each nucleotide position), resulting in a final sequence identified as SEQ ID NO: 1.

Therefore, SEQ ID NO:1 and the translated SEQ ID NO:2 are sufficiently accurate and otherwise suitable for a variety of uses well known in the art and described further below. For instance, SEQ ID NO:1 is useful for designing nucleic acid hybridization probes that will detect nucleic acid sequences contained in SEQ ID NO:1 or the cDNA contained in the deposited clone. These probes will also hybridize to nucleic acid molecules in biological samples, thereby enabling a variety of forensic and diagnostic methods of the invention. Similarly, polypeptides identified from SEQ ID NO:2 may be used to generate antibodies which bind specifically to CTGF-4.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly,(for those applications requiring precision in the nucleotide sequence or the amino acid sequence, the present invention provides not only the generated nucleotide sequence identified as SEQ ID NO:1 and the predicted translated amino acid sequence identified as SEQ ID NO:2, but also a sample of two overlapping plasmid DNAs each containing a human cDNA of CTGF-4 deposited with the ATCC. The nucleotide sequence of the deposited CTGF-4 clone can readily be determined by generating a single cDNA clone from the two in the deposit (for example, by overlap PCR) and then sequencing the deposited clone in accordance with known methods. More simply, each of the two clones in the deposit can be sequenced individually and a single CTGF-4 contig can be generated from the sequence information. The predicted CTGF-4 amino acid sequence can then be verified from such deposits. Moreover, the amino acid sequence of the protein encoded by the deposited clones can also be directly determined by peptide sequencing or by expressing the protein in a suitable host cell containing the deposited human CTGF-4 cDNAs, collecting the protein, and determining its sequence.

The present invention also relates to the CTGF-4 gene corresponding to SEQ ID NO:1, SEQ ID NO:2, or the deposited clones. The CTGF-4 gene can be isolated in accordance with known methods using the sequence information disclosed herein. Such methods include preparing probes or primers from the disclosed sequence and identifying or amplifying the CTGF-4 gene from appropriate sources of genomic material.

Also provided in the present invention are species homologs of CTGF-4. Species homologs may be isolated and identified by making suitable probes or primers from the sequences provided herein and screening a suitable nucleic acid source for the desired homolog.

The CTGF-4 polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The CTGF-4 polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

CTGF-4 polypeptides are preferably provided in an isolated form, and preferably are substantially purified. A recombinantly produced version of a CTGF-4 polypeptide, including the secreted polypeptide, can be substantially purified by the one-step method described in the publication by Smith and Johnson (Gene 67:31-40 (1988)). CTGF-4 polypeptides also can be purified from natural or recombinant sources using antibodies of the invention raised against the CTGF-4 protein in methods which are well known in the art.

Polynucleotide and Polypeptide Variants

“Variant” refers to a polynucleotide or polypeptide differing from the CTGF-4 polynucleotide or polypeptide, but retaining essential properties thereof. Generally, variants are overall closely similar, and, in many regions, identical to the CTGF-4 polynucleotide or polypeptide.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the CTGF-4 polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown of SEQ ID NO: 1, the ORF (open reading frame), or any fragment specified as described herein.

As a practical matter, whether any particular nucleic acid molecule or polypeptide is at least 90%, 95%, 96%, 97%, 98% or 99% identical to (or, as expressed in another way, at most 10%, 5%, 4%, 3%, 2% or 1% different from) a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag and colleagues (Comp. App. Biosci. 6:237-245 (1990)). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched or aligned, as a percent of the total bases of the query sequence. Whether a nucleotide is matched or aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched or aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

The CTGF-4 variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. CTGF-4 polynucleotide variants can be produced for a variety of reasons, e.g., to optimize codon expression for a particular host (change codons in the human mRNA to those preferred by a bacterial host such as E. coli).

Naturally occurring CTGF-4 variants are called “allelic variants”, and refer to one of several alternate forms of a gene occupying a given locus on a chromosome of an organism (Genes II, Lewin, B., ed., John Wiley & Sons, New York (1985)). These allelic variants can vary at either the polynucleotide or polypeptide level or at both the polynucleotide and polypeptide levels. Alternatively, non-naturally occurring variants may be produced by mutagenesis techniques or by direct synthesis.

Using known methods of protein engineering and recombinant DNA technology, variants may be generated to improve or alter the characteristics of the CTGF-4 polypeptides. For instance, one or more amino acids can be deleted from the N-terminus or C-terminus of the secreted protein without substantial loss of biological function. Ron and colleagues (J. Biol. Chem. 268:2984-2988 (1993)), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli, et al., J. Biotechnology 7:199-216 (1988)).

Moreover, ample evidence demonstrates that variants often retain a biological activity similar to that of the naturally occurring protein. For example, Gayle and coworkers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity]” (See, Abstract). In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes CTGF-4 polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided by Bowie and coworkers (Science 247:1306-1310 (1990)), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used (Cunningham and Wells, Science 244:1081-1085 (1989)). The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of CTGF-4 include: (i) substitutions with one or more of the non-conserved amino acid residues, where the substituted amino acid residues may or may not be one encoded by the genetic code, (ii) substitution with one or more of amino acid residues having a substituent group, (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretory sequence, or a sequence facilitating purification. Such variant polypeptides are deemed to be within the scope of those skilled in the art from the teachings herein.

For example, CTGF-4 polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity (Pinckard, et al., Clin. Exp. Immunol. 2:331-340 (1967); Robbins, et al., Diabetes 36:838-845 (1987); Cleland, et al., Crit. Rev. Therapeutic Drug Carrier Systems 10:307-377 (1993)).

As shown in FIGS. 2A, 2B, 2C, 2D, and 2E, CTGF-4 contains a number of highly conserved amino acid residues when compared to other members of the CCN family of growth factors. In addition to the highly conserved amino acid residues are shown as Conserved Domains I-XI in FIGS. 1A, 1B, 1C, and 1D, there are a number of specific amino acid residues which are highly conserved between many, if not all, CCN family members. As is well-known by those skilled in the art, such highly conserved amino acid residues are prime candidates for mutagenesis for the purposes of altering CTGF-4 function, producing a CTGF-4 protein with altered characteristics, and the like. A partial list of such highly conserved residues includes amino acids Leu-7, Cys-17, Cys-21, Cys-23, Pro-24, Pro-27, Pro-28, Cys-30, Gly-33, Val-34, Leu-36, Asp-39, Gly-40, Cys-41, Cys-43, Cys-44, Cys-47, Ala-48, Gln-50, Leu-51, Gly-52, Cys-55, Cys-60, Asp-61, Gly-65, Leu-66, Cys-69, Asp-70, Gly-81, Cys-83, Ala-85, Gly-89, Cys-91, Tyr-98, Gly-101, Ser-103, Phe-104, Gln-105, Cys-108, Lys-109, Cys-112, Thr-113, Cys-114, Asp-116, Gly-117, Val-119, Gly-120, Cys-121, Pro-123, Leu-124, Cys-125, Pro-131, Cys-135, Pro-136, Pro-138, Arg-139, Val-141, Pro-144, Gly-145, Cys-147, Cys-148, Glu-149, Trp-151, Val-152, Cys-153, Ala-170, Asn-183, Cys-184, Ile-185, Thr-188, Trp-191, Ser-192, Cys-194, Ser-195, Cys-198, Gly-199, Gly-201, Ser-203, Thr-204, Arg-205, Asn-208, Asn-210, Cys-213, Arg-220, Cys-222, Arg-225, Pro-226, Cys-227, Lys-236, Gly-238, Lys-239, Lys-240, Cys-241, Phe-253, Gly-257, Cys-258, Ser-260, Tyr-264, Pro-266, Lys-267, Cys-269, Gly-270, Val-271, Cys-272, Asp-274, Arg-276, Cys-277, Cys-278, Pro-280, Thr-285, Phe-290, Cys-292, Gly-295, Val-302, Ile-305, Cys-308, Cys-310, Cys-314, Asn-318, and Phe-321 of SEQ ID NO:2.

In addition, as is also well-known by those skilled in the art, amino acid residues which are potential targets for N-linked glycosylation are also prime candidates for mutagenesis for the purposes of altering CTGF-4 function, producing a CTGF-4 protein with altered characteristics, and the like. A partial list of amino acid residues which comprise potential N-linked glycosylation targets of a CTGF-4 polypeptide includes amino acids Asn-54, Cys-55, Thr-56, Asn-111, Cys-112, Thr-113, Asn-252, Phe-253, Thr-254, Asn-311, Leu-312, and Ser-313 of SEQ ID NO:2.

Polynucleotide and Polypeptide Fragments

In the present invention, a “polynucleotide fragment” refers to a short polynucleotide having a nucleic acid sequence contained in the deposited clone or shown in SEQ ID NO: 1. The short nucleotide fragments are preferably at least about 15 nt, and more preferably at least about 20 nt, still more preferably at least about 30 nt, and even more preferably, at least about 40 nt in length. A fragment “at least 20 nt in length”, for example, is intended to include 20 or more contiguous bases from the cDNA sequence contained in the deposited clone or the nucleotide sequence shown in SEQ ID NO:1. These nucleotide fragments are useful as diagnostic probes and primers as discussed herein. Of course, larger fragments (e.g., 50, 150, 500, 600, 2000 nucleotides) are preferred.

Moreover, representative examples of CTGF-4 polynucleotide fragments include, for example, fragments having a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of SEQ ID NO:1 or the cDNA contained in the deposited clone. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

The invention also provides nucleic acid molecules having nucleotide sequences related to extensive portions of SEQ ID NO:1 which have been determined from the following related cDNA clones: HSKXM68R (SEQ ID NO:7), HSKXM67R (SEQ ID NO:8), HAPAO05R (SEQ ID NO:9), HGBAV43R (SEQ ID NO:10), HCDAN77R (SEQ ID NO:ll), and HSKDP76R (SEQ ID NO:12).

Further, the invention includes a polynucleotide comprising any portion of at least about 25 nucleotides, preferably at least about 30 nucleotides, and even more preferably about 50 nucleotides, of SEQ ID NO:1 from residue 1 to 1600, 50 to 1600, 100 to 1600, 150 to 1600, 200 to 1600, 250 to 1600, 300 to 1600, 350 to 1600, 400 to 1600, 450 to 1600, 500 to 1600, 550 to 1600, 600 to 1600, 650 to 1600, 700 to 1600, 750 to 1600, 800 to 1600, 850 to 1600, 900 to 1600, 950 to 1600, 1000 to 1600, 1050 to 1600, 1100 to 1600, 1150 to 1600, 1200 to 1600, 1250 to 1600, 1300 to 1600, 1350 to 1600, 1400 to 1600, 1450 to 1600, 1500 to 1600, 1550 to 1600, 1 to 1550, 50 to 1550, 100 to 1550, 150 to 1550, 200 to 1550, 250 to 1550, 300 to 1550, 350 to 1550, 400 to 1550, 450 to 1550, 500 to 1550, 550 to 1550, 600 to 1550, 650 to 1550, 700 to 1550, 750 to 1550, 800 to 1550, 850 to 1550, 900 to 1550, 950 to 1550, 1000 to 1550, 1050 to 1550, 1100 to 1550, 1150 to 1550, 1200 to 1550, 1250 to 1550, 1300 to 1550, 1350 to 1550, 1400 to 1550, 1450 to 1550, 1500 to 1550, 1 to 1500, 50 to 1500, 100 to 1500, 150 to 1500, 200 to 1500, 250 to 1500, 300 to 1500, 350 to 1500, 400 to 1500, 450 to 1500, 500 to 1500, 550 to 1500, 600 to 1500, 650 to 1500, 700 to 1500, 750 to 1500, 800 to 1500, 850 to 1500, 900 to 1500, 950 to 1500, 1000 to 1500, 1050 to 1500, 1100 to 1500, 1150 to 1500, 1200 to 1500, 1250 to 1500, 1300 to 1500, 1350 to 1500, 1400 to 1500, 1450 to 1500, 1 to 1450, 50 to 1450, 100 to 1450, 150 to 1450, 200 to 1450, 250 to 1450, 300 to 1450, 350 to 1450, 400 to 1450, 450 to 1450, 500 to 1450, 550 to 1450, 600 to 1450, 650 to 1450, 700 to 1450, 750 to 1450, 800 to 1450, 850 to 1450, 900 to 1450, 950 to 1450, 1000 to 1450, 1050 to 1450, 1100 to 1450, 1150 to 1450, 1200 to 1450, 1250 to 1450, 1300 to 1450, 1350 to 1450, 1400 to 1450, 1 to 1400, 50 to 1400, 100 to 1400, 150 to 1400, 200 to 1400, 250 to 1400, 300 to 1400, 350 to 1400, 400 to 1400, 450 to 1400, 500 to 1400, 550 to 1400, 600 to 1400, 650 to 1400, 700 to 1400, 750 to 1400, 800 to 1400, 850 to 1400, 900 to 1400, 950 to 1400, 1000 to 1400, 1050 to 1400, 1100 to 1400, 1150 to 1400, 1200 to 1400, 1250 to 1400, 1300 to 1400, 1350 to 1400, 1 to 1350, 50 to 1350, 100 to 1350, 150 to 1350, 200 to 1350, 250 to 1350, 300 to 1350, 350 to 1350, 400 to 1350, 450 to 1350, 500 to 1350, 550 to 1350, 600 to 1350, 650 to 1350, 700 to 1350, 750 to 1350, 800 to 1350, 850 to 1350, 900 to 1350, 950 to 1350, 1000 to 1350, 1050 to 1350, 1100 to 1350, 1150 to 1350, 1200 to 1350, 1250 to 1350, 1300 to 1350, 1 to 1300, 50 to 1300, 100 to 1300, 150 to 1300, 200 to 1300, 250 to 1300, 300 to 1300, 350 to 1300, 400 to 1300, 450 to 1300, 500 to 1300, 550 to 1300, 600 to 1300, 650 to 1300, 700 to 1300, 750 to 1300, 800 to 1300, 850 to 1300, 900 to 1300, 950 to 1300, 1000 to 1300, 1050 to 1300, 1100 to 1300, 1150 to 1300, 1200 to 1300, 1250 to 1300, 1 to 1250, 50 to 1250, 100 to 1250, 150 to 1250, 200 to 1250, 250 to 1250, 300 to 1250, 350 to 1250, 400 to 1250, 450 to 1250, 500 to 1250, 550 to 1250, 600 to 1250, 650 to 1250, 700 to 1250, 750 to 1250, 800 to 1250, 850 to 1250, 900 to 1250, 950 to 1250, 1000 to 1250, 1050 to 1250, 1100 to 1250, 1150 to 1250, 1200 to 1250, 1 to 1200, 50 to 1200, 100 to 1200, 150 to 1200, 200 to 1200, 250 to 1200, 300 to 1200, 350 to 1200, 400 to 1200, 450 to 1200, 500 to 1200, 550 to 1200, 600 to 1200, 650 to 1200, 700 to 1200, 750 to 1200, 800 to 1200, 850 to 1200, 900 to 1200, 950 to 1200, 1000 to 1200, 1050 to 1200, 1100 to 1200, 1150 to 1200, 1 to 1150, 50 to 1150, 100 to 1150, 150 to 1150, 200 to 1150, 250 to 1150, 300 to 1150, 350 to 1150, 400 to 1150, 450 to 1150, 500 to 1150, 550 to 1150, 600 to 1150, 650 to 1150, 700 to 1150, 750 to 1150, 800 to 1150, 850 to 1150, 900 to 1150, 950 to 1150, 1000 to 1150, 1050 to 1150, 1100 to 1150, 1 to 1100, 50 to 1100, 100 to 1100, 150 to 1100, 200 to 1100, 250 to 1100, 300 to 1100, 350 to 1100, 400 to 1100, 450 to 1100, 500 to 1100, 550 to 1100, 600 to 1100, 650 to 1100, 700 to 1100, 750 to 1100, 800 to 1100, 850 to 1100, 900 to 1100, 950 to 1100, 1000 to 1100, 1050 to 1100, 1 to 1050, 50 to 1050, 100 to 1050, 150 to 1050, 200 to 1050, 250 to 1050, 300 to 1050, 350 to 1050, 400 to 1050, 450 to 1050, 500 to 1050, 550 to 1050, 600 to 1050, 650 to 1050, 700 to 1050, 750 to 1050, 800 to 1050, 850 to 1050, 900 to 1050, 950 to 1050, 1000 to 1050, 1 to 1000, 50 to 1000, 100 to 1000, 150 to 1000, 200 to 1000, 250 to 1000, 300 to 1000, 350 to 1000, 400 to 1000, 450 to 1000, 500 to 1000, 550 to 1000, 600 to 1000, 650 to 1000, 700 to 1000, 750 to 1000, 800 to 1000, 850 to 1000, 900 to 1000, 950 to 1000, 1 to 950, 50 to 950, 100 to 950, 150 to 950, 200 to 950, 250 to 950, 300 to 950, 350 to 950, 400 to 950, 450 to 950, 500 to 950, 550 to 950, 600 to 950, 650 to 950, 700 to 950, 750 to 950, 800 to 950, 850 to 950, 900 to 950, 1 to 900, 50 to 900, 100 to 900, 150 to 900, 200 to 900, 250 to 900, 300 to 900, 350 to 900, 400 to 900, 450 to 900, 500 to 900, 550 to 900, 600 to 900, 650 to 900, 700 to 900, 750 to 900, 800 to 900, 850 to 900, 1 to 850, 50 to 850, 100 to 850, 150 to 850, 200 to 850, 250 to 850, 300 to 850, 350 to 850, 400 to 850, 450 to 850, 500 to 850, 550 to 850, 600 to 850, 650 to 850, 700 to 850, 750 to 850, 800 to 850, 1 to 800, 50 to 800, 100 to 800, 150 to 800, 200 to 800, 250 to 800, 300 to 800, 350 to 800, 400 to 800, 450 to 800, 500 to 800, 550 to 800, 600 to 800, 650 to 800, 700 to 800, 750 to 800, 1 to 750, 50 to 750, 100 to 750, 150 to 750, 200 to 750, 250 to 750, 300 to 750, 350 to 750, 400 to 750, 450 to 750, 500 to 750, 550 to 750, 600 to 750, 650 to 750, 700 to 750, 1 to 700, 50 to 700, 100 to 700, 150 to 700, 200 to 700, 250 to 700, 300 to 700, 350 to 700, 400 to 700, 450 to 700, 500 to 700, 550 to 700, 600 to 700, 650 to 700, 1 to 650, 50 to 650, 100 to 650, 150 to 650, 200 to 650, 250 to 650, 300 to 650, 350 to 650, 400 to 650, 450 to 650, 500 to 650, 550 to 650, 600 to 650, 1 to 600, 50 to 600, 100 to 600, 150 to 600, 200 to 600, 250 to 600, 300 to 600, 350 to 600, 400 to 600, 450 to 600, 500 to 600, 550 to 600, 1 to 550, 50 to 550, 100 to 550, 150 to 550, 200 to 550, 250 to 550, 300 to 550, 350 to 550, 400 to 550, 450 to 550, 500 to 550, 1 to 500, 50 to 500, 100 to 500, 150 to 500, 200 to 500, 250 to 500, 300 to 500, 350 to 500, 400 to 500, 450 to 500, 1 to 450, 50 to 450, 100 to 450, 150 to 450, 200 to 450, 250 to 450, 300 to 450, 350 to 450, 400 to 450, 1 to 400, 50 to 400, 100 to 400, 150 to 400, 200 to 400, 250 to 400, 300 to 400, 350 to 400, 1 to 350, 50 to 350, 100 to 350, 150 to 350, 200 to 350, 250 to 350, 300 to 350, 1 to 300, 50 to 300, 100 to 300, 150 to 300, 200 to 300, 250 to 300, 1 to 250, 50 to 250, 100 to 250, 150 to 250, 200 to 250, 1 to 200, 50 to 200, 100 to 200, 150 to 200, 1 to 150, 50 to 150, 100 to 150, 1 to 100, 50 to 100, and 1 to 50.

In the present invention, a “polypeptide fragment” refers to a short amino acid sequence contained in SEQ ID NO:2 or encoded by the cDNA contained in the deposited clone. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, 141-160, 161-180, 181-200, 201-220, 221-240, 241-260, 261-280, or 281 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

A further embodiment of the invention relates to a peptide or polypeptide which comprises the amino acid sequence of an CTGF-4 polypeptide having an amino acid sequence which contains at least one conservative amino acid substitution, but not more than 50 conservative amino acid substitutions, even more preferably, not more than 40 conservative amino acid substitutions, still more preferably, not more than 30 conservative amino acid substitutions, and still even more preferably, not more than 20 conservative amino acid substitutions. Of course, in order of ever-increasing preference, it is highly preferable for a peptide or polypeptide to have an amino acid sequence which comprises the amino acid sequence of an CTGF-4 polypeptide, which contains at least one, but not more than 10, 9, 8, 7, 6, 5, 4, 3, 2 or 1 conservative amino acid substitutions.

Preferred polypeptide fragments include the secreted CTGF-4 protein as well as the mature form, the IGF-binding domain, the von Willebrand factor type C repeat domain, the sulfated glycoconjugate-binding motif, C-terminal dimerization and receptor-binding domain, and any of conserved domains I-XI (see above). Further preferred polypeptide fragments include the secreted CTGF-4 protein, the mature form, the IGF-binding domain, the von Willebrand factor type C repeat domain, the sulfated glycoconjugate-binding motif, and the C-terminal dimerization and receptor-binding domain, having a continuous series of deleted residues from the amino or the carboxy terminus, or both. For example, any number of amino acids, ranging from 1-60, can be deleted from the amino terminus of the secreted CTGF-4 polypeptide, the mature form, the IGF-binding domain, the von Willebrand factor type C repeat domain, the sulfated glycoconjugate-binding motif or the C-terminal dimerization and receptor-binding domain. Similarly, any number of amino acids, ranging from 1-30, can be deleted from the carboxy terminus of the secreted CTGF-4 protein, the mature form, the IGF-binding domain, the von Willebrand factor type C repeat domain, the sulfated glycoconjugate-binding motif or the C-terminal dimerization and receptor-binding domain. Furthermore, any combination of the above amino and carboxy terminus deletions are preferred. Similarly, polynucleotide fragments encoding these CTGF-4 polypeptide fragments are also preferred.

Brigstock and colleagues (J. Biol. Chem. 272(32):20275-20282 (1998)) have isolated biologically active subfragments of the porcine heparin-binding growth factor (HBGF), a member of the CCN growth factor family, and, thus, a homolog of CTGF-4. The full-length HBGF polypeptide has a predicted molecular mass of approximately 38 kDa. However, Brigstock and coworkers (supra) isolated several subfragments thereof from heparin-binding fractions of pig uterine luminal flushings. The apparent molecular masses of the subfragments were 10, 16, and 20 kDa. The sequence identity of each of the subfragments was identified by N-terminal sequencing of isolated polypeptides. Of the subfragments, the 10 kDa fragment retained the ability to stimulate fibroblast DNA synthesis. The high level of sequence identity between CTGF-4, other members of the CCN family, and HBGF, and the abovementioned observations suggest that CTGF-4 may also be processed beyond cleavage of the secretory signal peptide.

An alignment of the sequences identifies specific regions of the CTGF-4 polypeptide which may possess a similar biological activity. A polypeptide fragment comprising amino acids residues 241-335 of the CTGF-4 amino acid sequence shown as SEQ ID NO:2 corresponds to the biologically active 10 kDa HBGF subfragment identified by Brigstock and colleagues (supra). As such, it is likely that a polypeptide fragment comprising amino acids residues 241-335 of the CTGF-4 amino acid sequence shown as SEQ ID NO:2 will retain a highly similar ability to affect the synthesis of DNA in fibroblasts (however, this is not to suggest that a polypeptide fragment comprising amino acids residues 241-335 of the CTGF-4 amino acid sequence shown as SEQ ID NO:2 will retain all biological properties and activities of the full-length or of the mature CTGF-4 polypeptides). However, based on sequence conservation of several members of the CCN growth factor family (see CD-VIII in FIGS. 2A, 2B, 2C, 2D, and 2E), it is likely that amino acid residues H-241, T-242, L-243, and 1-244 may be removed from the CTGF-4 amino acid residues 241-335 subfragment without significantly affecting activity of the fragment. Thus, subfragments of the CTGF-4 polypeptide comprising the sequence shown in SEQ ID NO:2 from amino acid residues 232-335, 233-335, 234-335, 235-335, and 236-335 are expected to retain a highly similar ability to affect the synthesis of DNA in fibroblasts.

Likewise (although the C-terminus of the HBGF 10 kDa subfragments were not determined), subfragments of the CTGF-4 polypeptide comprising the sequence shown in SEQ ID NO:2 from amino acid residues 232-335, 232-334, 232-333, 232-332, 232-331, 232-330, 232-329, 232-328, 232-327, 232-326, 232-325, 232-324, 232-323, 232-322, 232-321, 232-320, 232-319, 232-318, 232-317, 232-316, 232-315, and 236-314 are expected to retain a highly similar ability to affect the synthesis of DNA in fibroblasts.

Moreover, the invention also provides polypeptides having one or more amino acids deleted from the amino-terminus (i.e. residues 231-235 may be deleted as described above) and carboxy-terminus (i.e. residues 315-335 may be deleted as described above) of a polypeptide fragment comprising amino acids residues 241-335 of the CTGF-4 amino acid sequence shown as SEQ ID NO:2. As described above, it is expected that such an N- and C-terminal deletion mutein will retain all biological properties and activities of the full-length or of the mature CTGF-4 polypeptides. In fact, two independent 10 kDa HBGF subfragment species, differing by a single amino acid residue at the N-terminus, have been isolated and exhibit highly similar abilities to affect the synthesis of DNA in fibroblasts.

More particularly, N-terminal deletion mutations of the CTGF-4 polypeptide can be described by the general formula “m-343”, where “m” is an integer from 2 to 338 corresponding to the position of the amino acid identified in SEQ ID NO:2. In the following list, the variable “m” is also associated with the single letter amino acid abbreviation for the residue at that position (for example, where “m” is to represent position 2 of SEQ ID NO:2, “m” is shown as “F-2” in the following list). Preferably, N-terminal deletions of the CTGF-4 polypeptide of the invention shown as SEQ ID NO:2 include polypeptides comprising, or alternatively consisting of, the amino acid sequence of the following list of residues having value m-343: F-2 to N-343; T-3 to N-343; P-4 to N-343; A-5 to N-343; P-6 to N-343; L-7 to N-343; E-8 to N-343; D-9 to N-343; T-10 to N-343; S-11 to N-343; S-12 to N-343; R-13 to N-343; P-14 to N-343; Q-15 to N-343; F-16 to N-343; C-17 to N-343; K-18 to N-343; W-19 to N-343; P-20 to N-343; C-21 to N-343; E-22 to N-343; C-23 to N-343; P-24 to N-343; P-25 to N-343; S-26 to N-343; P-27 to N-343; P-28 to N-343; R-29 to N-343; C-30 to N-343; P-31 to N-343; L-32 to N-343; G-33 to N-343; V-34 to N-343; S-35 to N-343; L-36 to N-343; 1-37 to N-343; T-38 to N-343; D-39 to N-343; G-40 to N-343; C-41 to N-343; E-42 to N-343; C-43 to N-343; C-44 to N-343; K-45 to N-343; M-46 to N-343; C-47 to N-343; A-48 to N-343; Q-49 to N-343; Q-50 to N-343; L-51 to N-343; G-52 to N-343; D-53 to N-343; N-54 to N-343; C-55 to N-343; T-56 to N-343; E-57 to N-343; A-58 to N-343; A-59 to N-343; 1-60 to N-343; C-61 to N-343; D-62 to N-343; P-63 to N-343; H-64 to N-343; R-65 to N-343; G-66 to N-343; L-67 to N-343; Y-68 to N-343; C-69 to N-343; D-70 to N-343; Y-71 to N-343; S-72 to N-343; G-73 to N-343; D-74 to N-343; R-75 to N-343; P-76 to N-343; R-77 to N-343; Y-78 to N-343; A-79 to N-343; 1-80 to N-343; -81 to N-343; -82 to N-343; G-83 to N-343; V-84 to N-343; C-85 to N-343; A-86 to N-343; Q-87 to N-343; V-88 to N-343; V-89 to N-343; G-90 to N-343; V-91 to N-343; G-92 to N-343; C-93 to N-343; V-94 to N-343; L-95 to N-343; D-96 to N-343; G-97 to N-343; V-98 to N-343; R-99 to N-343; Y-100 to N-343; N-101 to N-343; N-102 to N-343; G-103 to N-343; Q-104 to N-343; S-105 to N-343; F-106 to N-343; Q-107 to N-343; P-108 to N-343; N-109 to N-343; C-110 to N-343; K-111 to N-343; Y-112, to N-343; N-113 to N-343; C-114 to N-343; T-115 to N-343; C-116 to N-343; 1-117 to N-343; D-118 to N-343; G-119 to N-343; A-120 to N-343; V-121 to N-343; G-122 to N-343; C-123 to N-343; T-124 to N-343; P-125 to N-343; L-126 to N-343; C-127 to N-343; L-128 to N-343; R-129 to N-343; V-130 to N-343; R-131 to N-343; P-132 to N-343; P-133 to N-343; R-134 to N-343; L-135 to N-343; W-136 to N-343; C-137 to N-343; P-138 to N-343; H-139 to N-343; P-140 to N-343; R-141 to N-343; R-142 to N-343; V-143 to N-343; S-144 to N-343; 1-145 to N-343; P-146 to N-343; G-147 to N-343; H-148 to N-343; C-149 to N-343; C-150 to N-343; E-151 to N-343; Q-152 to N-343; W-153 to N-343; V-154 to N-343; C-155 to N-343; E-156 to N-343; D-157 to N-343; D-158 to N-343; A-159 to N-343; K-160 to N-343; R-161 to N-343; P-162 to N-343; -163 to N-343; -164 to N-343; R-165 to N-343; K-166 to N-343; T-167 to N-343; A-168 to N-343; P-169 to N-343; R-170 to N-343; D-171 to N-343; T-172 to N-343; G-173 to N-343; A-174 to N-343; F-175 to N-343; D-176 to N-343; A-177 to N-343; V-178 to N-343; G-179 to N-343; E-180 to N-343; V-181 to N-343; E-182 to N-343; A-183 to N-343; W-184 to N-343; H-185 to N-343; R-186 to N-343; N-187 to N-343; C-188 to N-343; 1-189 to N-343; A-190 to N-343; Y-191 to N-343; T-192 to N-343; S-193 to N-343; P-194 to N-343; W-195 to N-343; S-196 to N-343; P-197 to N-343; C-198 to N-343; S-199 to N-343; T-200 to N-343; S-201 to N-343; C-202 to N-343; G-203 to N-343; L-204 to N-343; G-205 to N-343; V-206 to N-343; S-207 to N-343; T-208 to N-343; R-209 to N-343; I-210 to N-343; S-211 to N-343; N-212 to N-343; V-213 to N-343; N-214 to N-343; A-215 to N-343; Q-216 to N-343; C-217 to N-343; W-218 to N-343; P-219 to N-343; E-220 to N-343; Q-221 to N-343; E-222 to N-343; S-223 to N-343; R-224 to N-343; L-225 to N-343; C-226 to N-343; N-227 to N-343; L-228 to N-343; R-229 to N-343; P-230 to N-343; C-231 to N-343; D-232 to N-343; V-233 to N-343; D-234 to N-343; I-235 to N-343; H-236 to N-343; T-237 to N-343; L-238 to N-343; I-239 to N-343; K-240 to N-343; A-241 to N-343; G-242 to N-343; K-243 to N-343; K-244 to N-343; -245 to N-343; -246 to N-343; C-247 to N-343; L-248 to N-343; A-249 to N-343; V-250 to N-343; Y-251 to N-343; Q-252 to N-343; P-253 to N-343; E-254 to N-343; A-255 to N-343; S-256 to N-343; M-257 to N-343; N-258 to N-343; F-259 to N-343; T-260 to N-343; L-261 to N-343; A-262 to N-343; G-263 to N-343; C-264 to N-343; I-265 to N-343; S-266 to N-343; T-267 to N-343; R-268 to N-343; S-269 to N-343; Y-270 to N-343; Q-271 to N-343; P-272 to N-343; K-273 to N-343; Y-274 to N-343; C-275 to N-343; G-276 to N-343; V-277 to N-343; C-278 to N-343; M-279 to N-343; D-280 to N-343; N-281 to N-343; R-282 to N-343; C-283 to N-343; C-284 to N-343; 1-285 to N-343; P-286 to N-343; Y-287 to N-343; K-288 to N-343; S-289 to N-343; K-290 to N-343; T-291 to N-343; I-292 to N-343; D-293 to N-343; V-294 to N-343; S-295 to N-343; F-296 to N-343; Q-297 to N-343; C-298 to N-343; P-299 to N-343; D-300 to N-343; G-301 to N-343; L-302 to N-343; G-303 to N-343; F-304 to N-343; S-305 to N-343; R-306 to N-343; Q-307 to N-343; V-308 to N-343; L-309 to N-343; W-310 to N-343; 1-311 to N-343; N-312 to N-343; A-313 to N-343; C-314 to N-343; F-315 to N-343; C-316 to N-343; N-317 to N-343; L-318 to N-343; S-319 to N-343; C-320 to N-343; R-321 to N-343; N-322 to N-343; P-323 to N-343; N-324 to N-343; D-325 to N-343; 1-326 to N-343; -327 to N-343; -328 to N-343; F-329 to N-343; A-330 to N-343; D-331 to N-343; L-332 to N-343; E-333 to N-343; S-334 to N-343; Y-335 to N-343; P-336 to N-343; D-337 to N-343; F-338 to N-343 of SEQ ID NO:2. Polynucleotides encoding these polypeptides are also provided. The present application is also directed to nucleic acid molecules comprising, or alternatively, consisting of, a polynucleotide sequence at least 90%, 95%, 96%, 97%, 98% or 99% identical to the polynucleotide sequence encoding the CTGF-4 polypeptides described above. The present invention also encompasses the above polynucleotide sequences fused to a heterologous polynucleotide sequence.

Particularly, C-terminal deletion mutations of the CTGF-4 polypeptide can be described by the general formula “1-n”, where “n” is an integer from 6 to 342 corresponding to the position of the amino acid identified in SEQ ID NO:2. In the following list, the variable “n” is also associated with the single letter amino acid abbreviation for the residue at that position (for example, where “n” is to represent position 342 of SEQ ID NO:2, “n” is shown as “A-342” in the following list). Preferably, C-terminal deletions of the CTGF-4 polypeptide of the invention shown as SEQ ID NO:2 include polypeptides comprising, or alternatively consisting of, the amino acid sequence of the following list of residues having value 1-n: D-1 to A-342; D-1 to 1-341; D-1 to E-340; D-1 to S-339; D-1 to F-338; D-1 to D-337; D-1 to P-336; D-1 to Y-335; D-1 to S-334; D-1 to E-333; D-1 to L-332; D-1 to D-331; D-1 to A-330; D-1 to F-329; D-1 to -328; D-1 to -327; D-1 to 1-326; D-1 to D-325; D-1 to N-324; D-1 to P-323; D-1 to N-322; D-1 to R-321; D-1 to C-320; D-1 to S-319; D-1 to L-318; D-1 to N-317; D-1 to C-316; D-1 to F-315; D-1 to C-314; D-1 to A-313; D-1 to N-312; D-1 to I-311; D-1 to W-310; D-1 to L-309; D-1 to V-308; D-1 to Q-307; D-1 to R-306; D-1 to S-305; D-1 to F-304; D-1 to G-303; D-1 to L-302; D-1 to G-301; D-1 to D-300; D-1 to P-299; D-1 to C-298; D-1 to Q-297; D-1 to F-296; D-1 to S-295; D-1 to V-294; D-1 to D-293; D-1 to 1-292; D-1 to T-291; D-1 to K-290; D-1 to S-289; D-1 to K-288; D-1 to Y-287; D-1 to P-286; D-1 to 1-285; D-1 to C-284; D-1 to C-283; D-1 to R-282; D-1 to N-281; D-1 to D-280; D-1 to M-279; D-1 to C-278; D-1 to V-277; D-1 to G-276; D-1 to C-275; D-1 to Y-274; D-1 to K-273; D-1 to P-272; D-1 to Q-271; D-1 to Y-270; D-1 to S-269; D-1 to R-268; D-1 to T-267; D-1 to S-266; D-1 to 1-265; D-1 to C-264; D-1 to G-263; D-1 to A-262; D-1 to L-261; D-1 to T-260; D-1 to F-259; D-1 to N-258; D-1 to M-257; D-1 to S-256; D-1 to A-255; D-1 to E-254; D-1 to P-253; D-1 to Q-252; D-1 to Y-251; D-1 to V-250; D-1 to A-249; D-1 to L-248; D-1 to C-247; D-1 to −246; D-1 to −245; D-1 to K-244; D-1 to K-243; D-1 to G-242; D-1 to A-241; D-1 to K-240; D-1 to 1-239; D-1 to L-238; D-1 to T-237; D-1 to H-236; D-1 to 1-235; D-1 to D-234; D-1 to V-233; D-1 to D-232; D-1 to C-231; D-1 to P-230; D-1 to R-229; D-1 to L-228; D-1 to N-227; D-1 to C-226; D-1 to L-225; D-1 to R-224; D-1 to S-223; D-1 to E-222; D-1 to Q-221; D-1 to E-220; D-1 to P-219; D-1 to W-218; D-1 to C-217; D-1 to Q-216; D-1 to A-215; D-1 to N-214; D-1 to V-213; D-1 to N-212; D-1 to S-211; D-1 to 1-210; D-1 to R-209; D-1 to T-208; D-1 to S-207; D-1 to V-206; D-1 to G-205; D-1 to L-204; D-1 to G-203; D-1 to C-202; D-1 to S-201; D-1 to T-200; D-1 to S-199; D-1 to C-198; D-1 to P-197; D-1 to S-196; D-1 to W-195; D-1 to P-194; D-1 to S-193; D-1 to T-192; D-1 to Y-191; D-1 to A-190; D-1 to 1-189; D-1 to C-188; D-1 to N-187; D-1 to R-186; D-1 to H-185; D-1 to W-184; D-1 to A-183; D-1 to E-182; D-1 to V-181; D-1 to E-180; D-1 to G-179; D-1 to V-178; D-1 to A-177; D-1 to D-176; D-1 to F-175; D-1 to A-174; D-1 to G-173; D-1 to T-172; D-1 to D-171; D-1 to R-170; D-1 to P-169; D-1 to A-168; D-1 to T-167; D-1 to K-166; D-1 to R-165; D-1 to −164; D-1 to −163; D-1 to P-162; D-1 to R-161; D-1 to K-160; D-1 to A-159; D-1 to D-158; D-1 to D-157; D-1 to E-156; D-1 to C-155; D-1 to V-154; D-1 to W-153; D-1 to Q-152; D-1 to E-151; D-1 to C-150; D-1 to C-149; D-1 to H-148; D-1 to G-147; D-1 to P-146; D-1 to 1-145; D-1 to S-144; D-1 to V-143; D-1 to R-142; D-1 to R-141; D-1 to P-140; D-1 to H-139; D-1 to P-138; D-1 to C-137; D-1 to W-136; D-1 to L-135; D-1 to R-134; D-1 to P-133; D-1 to P-132; D-1 to R-131; D-1 to V-130; D-1 to R-129; D-1 to L-128; D-1 to C-127; D-1 to L-126; D-1 to P-125; D-1 to T-124; D-1 to C-123; D-1 to G-122; D-1 to V-121; D-1 to A-120; D-1 to G-119; D-1 to D-118; D-1 to I-117; D-1 to C-116; D-1 to T-115; D-1 to C-114; D-1 to N-113; D-1 to Y-112; D-1 to K-111; D-1 to C-110; D-1 to N-109; D-1 to P-108; D-1 to Q-107; D-1 to F-106; D-1 to S-105; D-1 to Q-104; D-1 to G-103; D-1 to N-102; D-1 to N-101; D-1 to Y-100; D-1 to R-99; D-1 to V-98; D-1 to G-97; D-1 to D-96; D-1 to L-95; D-1 to V-94; D-1 to C-93; D-1 to G-92; D-1 to V-91; D-1 to G-90; D-1 to V-89; D-1 to V-88; D-1 to Q-87; D-1 to A-86; D-1 to C-85; D-1 to V-84; D-1 to G-83; D-1 to −82; D-1 to −81; D-1 to 1-80; D-1 to A-79; D-1 to Y-78; D-1 to R-77; D-1 to P-76; D-1 to R-75; D-1 to D-74; D-1 to G-73; D-1 to S-72; D-1 to Y-71; D-1 to D-70; D-1 to C-69; D-1 to Y-68; D-1 to L-67; D-1 to G-66; D-1 to R-65; D-1 to H-64; D-1 to P-63, D-1 to D-62; D-1 to C-61; D-1 to 1-60; D-1 to A-59; D-1 to A-58; D-1 to E-57; D-1 to T-56; D-1 to C-55; D-1 to N-54; D-1 to D-53; D-1 to G-52; D-1 to L-51; D-1 to Q-50; D-1 to Q-49; D-1 to A-48; D-1 to C-47; D-1 to M-46; D-1 to K-45; D-1 to C-44; D-1 to C-43; D-1 to E-42; D-1 to C-41; D-1 to G-40; D-1 to D-39; D-1 to T-38; D-1 to 1-37; D-1 to L-36; D-1 to S-35; D-1 to V-34; D-1 to G-33; D-1 to L-32; D-1 to P-31; D-1 to C-30; D-1 to R-29; D-1 to P-28; D-1 to P-27; D-1 to S-26; D-1 to P-25; D-1 to P-24; D-1 to C-23; D-1 to E-22; D-1 to C-21; D-1 to P-20; D-1 to W-19; D-1 to K-18; D-1 to C-17; D-1 to F-16; D-1 to Q-15; D-i to P-14; D-1 to R-13; D-1 to S-12; D-1 to S-11; D-1 to T-10; D-1 to D-9; D-1 to E-8; D-1 to L-7; and D-1 to P-6 of SEQ ID NO:2. Polynucleotides encoding these polypeptides are also provided. The present application is also directed to nucleic acid molecules comprising, or alternatively, consisting of, a polynucleotide sequence at least 90%, 95%, 96%, 97%, 98% or 99% identical to the polynucleotide sequence encoding the CTGF-4 polypeptides described above. The present invention also encompasses the above polynucleotide sequences fused to a heterologous polynucleotide sequence.

The invention also provides polypeptides having one or more amino acids deleted from both the amino- and carboxy-termini, which may be described generally as comprising residues n-m of SEQ ID NO:2, where n and m are integers as described above.

Also preferred are CTGF-4 polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of SEQ ID NO:2 falling within conserved domains are specifically contemplated by the present invention (See FIGS. 2A, 2B, 2C, 2D, and 2E). Moreover, polynucleotide fragments encoding these domains are also contemplated.

In additional embodiments, the polynucleotides of the invention encode functional attributes of CTGF-4. Preferred embodiments of the invention in this regard include fragments that comprise alpha-helix and alpha-helix forming regions (“alpha-regions”), beta-sheet and beta-sheet forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions and high antigenic index regions of CTGF-4.

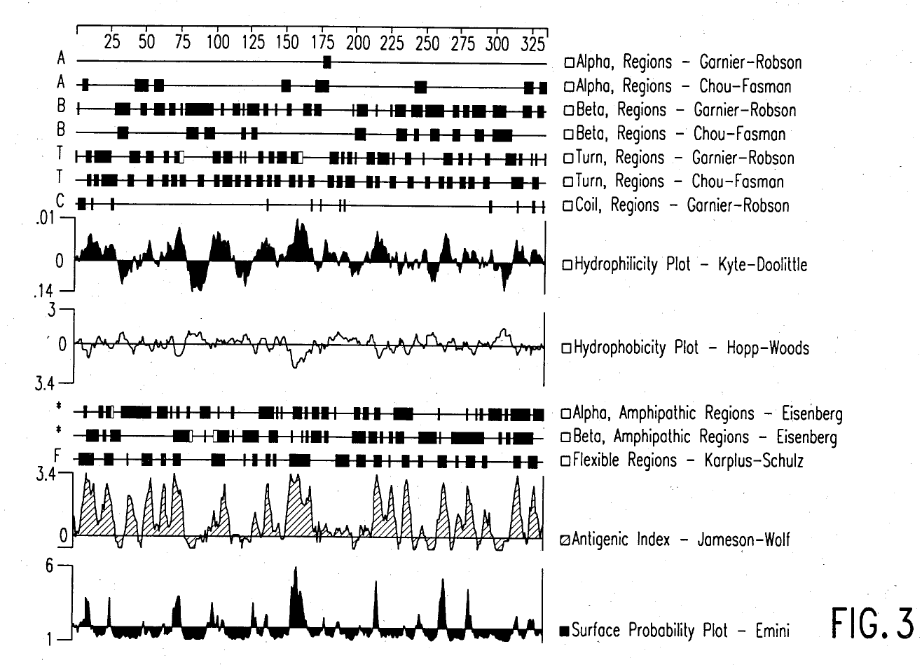

The data representing the structural or functional attributes of CTGF-4 set forth in FIG. 3 and/or Table I, as described above, was generated using the various modules and algorithms of the DNA*STAR set on default parameters. In a preferred embodiment, the data presented in columns VIH, IX, XIH, and XIV of Table I can be used to determine regions of CTGF-4 which exhibit a high degree of potential for antigenicity. Regions of high antigenicity are determined from the data presented in columns VIII, IX, XIII, and/or IV by choosing values which represent regions of the polypeptide which are likely to be exposed on the surface of the polypeptide in an environment in which antigen recognition may occur in the process of initiation of an immune response.

Certain preferred regions in these regards are set out in FIG. 3, but may, as shown in Table I, be represented or identified by using tabular representations of the data presented in FIG. 3. The DNA*STAR computer algorithm used to generate FIG. 3 (set on the original default parameters) was used to present the data in FIG. 3 in a tabular format (See Table I). The tabular format of the data in FIG. 3 may be used to easily determine specific boundaries of a preferred region.

The above-mentioned preferred regions set out in FIG. 3, and in Table I, respectively, include, but are not limited to, regions of the aforementioned types identified by analysis of the amino acid sequence set out in FIGS. 1A, 1B, 1C, and 1D. As set out in FIGS. 1A, 1B, 1C, and 1D, and in Table I, such preferred regions include Garnier-Robson alpha-regions, beta-regions, turn-regions, and coil-regions, Chou-Fasman alpha-regions, beta-regions, and coil-regions, Kyte-Doolittle hydrophilic regions and hydrophobic regions, Eisenberg alpha- and beta-amphipathic regions, Karplus-Schulz flexible regions, Emini surface-forming regions and Jameson-Wolf regions of high antigenic index.

TABLE I

| Res. | Pos. | I | II | III | IV | V | VI | VII | VIII | IX | X | XI | XII | XIII | XIV |
|---|---|---|---|---|---|---|---|---|---|---|---|---|---|---|---|
| Asp | 1 | . | . | . | . | T | . | . | 0.13 | −0.01 | . | . | . | 1.05 | 1.21 |
| Phe | 2 | . | . | B | . | . | . | . | 0.31 | 0.06 | . | . | . | −0.10 | 0.96 |
| Thr | 3 | . | . | . | . | . | . | C | −0.11 | 0.06 | . | . | . | 0.25 | 1.16 |
| Pro | 4 | . | . | . | . | . | . | C | 0.28 | 0.31 | . | . | . | 0.10 | 0.57 |
| Ala | 5 | . | A | . | . | . | . | C | 0.67 | 0.31 | . | . | F | 0.54 | 1.14 |
| Pro | 6 | . | A | . | . | . | . | C | 0.36 | −0.47 | . | . | F | 1.48 | 1.32 |
| Leu | 7 | . | A | . | . | . | . | C | 0.76 | −0.47 | . | . | F | 1.82 | 1.23 |
| Glu | 8 | . | A | . | . | T | . | . | 0.77 | −0.51 | * | . | F | 2.66 | 1.64 |
| Asp | 9 | . | . | . | . | T | T | . | 1.09 | −0.63 | * | . | F | 3.40 | 1.42 |
| Thr | 10 | . | . | . | . | T | T | . | 1.47 | −1.06 | . | * | F | 3.06 | 3.37 |
| Ser | 11 | . | . | . | . | T | T | . | 1.68 | −1.31 | . | * | F | 3.00 | 3.01 |
| Ser | 12 | . | . | . | . | T | T | . | 1.79 | −0.91 | . | * | F | 2.94 | 3.12 |
| Arg | 13 | . | . | . | . | . | . | C | 1.12 | −0.13 | . | * | F | 2.18 | 1.87 |
| Pro | 14 | . | . | . | . | T | T | . | 1.17 | −0.04 | . | * | F | 2.37 | 0.75 |
| Gln | 15 | . | . | . | . | T | T | . | 1.19 | −0.43 | . | * | F | 2.80 | 1.12 |
| Phe | 16 | . | . | . | . | T | T | . | 1.28 | 0.10 | . | * | . | 1.62 | 0.60 |
| Cys | 17 | . | . | . | . | T | T | . | 0.91 | 0.53 | . | * | . | 1.04 | 0.60 |
| Lys | 18 | . | . | . | . | T | . | . | 0.80 | 0.67 | . | * | . | 0.56 | 0.19 |
| Trp | 19 | . | . | . | . | T | T | . | 0.34 | 0.27 | * | . | . | 0.78 | 0.37 |
| Pro | 20 | . | . | . | . | T | T | . | 0.13 | 0.06 | * | . | . | 0.78 | 0.37 |
| Cys | 21 | . | . | . | . | T | T | . | 0.62 | −0.09 | * | . | . | 1.66 | 0.29 |
| Glu | 22 | . | . | . | . | T | T | . | 0.99 | 0.34 | . | * | . | 1.34 | 0.42 |
| Cys | 23 | . | . | . | . | T | T | . | 0.73 | −0.19 | . | * | F | 2.37 | 0.37 |
| Pro | 24 | . | . | . | . | T | T | . | 0.81 | −0.19 | * | . | F | 2.80 | 1.05 |
| Pro | 25 | . | . | . | . | T | T | . | 1.13 | −0.33 | * | . | F | 2.37 | 0.94 |
| Ser | 26 | . | . | . | . | . | T | C | 1.13 | −0.33 | * | . | F | 2.04 | 3.44 |
| Pro | 27 | . | . | . | . | . | T | C | 0.92 | −0.33 | * | * | F | 1.76 | 1.19 |
| Pro | 28 | . | . | . | . | T | T | . | 0.78 | −0.33 | . | * | F | 1.68 | 1.19 |
| Arg | 29 | . | . | B | . | . | T | . | 0.64 | −0.07 | * | * | F | 0.85 | 0.73 |
| Cys | 30 | . | . | B | . | . | T | . | −0.00 | −0.03 | . | * | . | 0.70 | 0.47 |
| Pro | 31 | . | . | B | B | . | . | . | −0.00 | 0.19 | . | * | . | −0.30 | 0.23 |
| Leu | 32 | . | . | B | B | . | . | . | −0.60 | 0.14 | . | * | . | −0.30 | 0.15 |
| Gly | 33 | . | . | B | B | . | . | . | −1.28 | 0.83 | . | * | . | −0.60 | 0.24 |
| Val | 34 | . | . | B | B | . | . | . | −1.70 | 0.94 | . | * | . | −0.60 | 0.11 |
| Ser | 35 | . | . | B | B | . | . | . | −1.03 | 1.00 | * | . | . | −0.60 | 0.19 |
| Leu | 36 | . | . | B | B | . | . | . | −1.17 | 0.31 | * | . | . | −0.08 | 0.32 |
| Ile | 37 | . | . | B | B | . | . | . | −1.02 | 0.31 | * | . | . | 0.14 | 0.42 |
| Thr | 38 | . | . | B | . | . | T | . | −0.68 | 0.24 | * | . | . | 0.76 | 0.17 |
| Asp | 39 | . | . | . | . | T | T | . | −0.49 | −0.14 | * | . | F | 2.13 | 0.36 |
| Gly | 40 | . | . | . | . | T | T | . | −0.86 | −0.26 | * | . | . | 2.20 | 0.27 |
| Cys | 41 | . | . | . | . | T | T | . | −0.00 | −0.37 | * | . | . | 1.98 | 0.10 |
| Glu | 42 | . | A | . | . | T | . | . | 0.29 | −0.86 | * | . | . | 1.66 | 0.12 |
| Cys | 43 | . | A | . | . | T | . | . | −0.07 | −0.24 | * | . | . | 1.14 | 0.12 |
| Cys | 44 | . | A | . | . | T | . | . | −0.66 | −0.10 | * | . | . | 0.92 | 0.12 |
| Lys | 45 | . | A | . | . | T | . | . | −0.31 | −0.17 | * | . | . | 0.70 | 0.07 |
| Met | 46 | . | A | B | . | . | . | . | 0.36 | 0.23 | * | . | . | −0.30 | 0.23 |
| Cys | 47 | . | A | B | . | . | . | . | −0.46 | 0.06 | * | . | . | −0.30 | 0.74 |
| Ala | 48 | . | A | B | . | . | . | . | −0.13 | 0.17 | * | . | . | −0.30 | 0.30 |
| Gln | 49 | . | A | B | . | . | . | . | 0.53 | 0.60 | * | . | . | −0.60 | 0.30 |
| Gln | 50 | . | A | B | . | . | . | . | 0.49 | −0.01 | * | . | F | 0.45 | 0.95 |
| Leu | 51 | . | A | . | . | T | . | . | 0.42 | −0.19 | * | . | F | 1.31 | 1.51 |
| Gly | 52 | . | . | . | . | T | T | . | 0.78 | −0.11 | * | . | F | 1.87 | 0.47 |
| Asp | 53 | . | . | . | . | T | T | . | 1.37 | −0.03 | * | . | F | 2.18 | 0.39 |
| Asn | 54 | . | . | . | . | T | T | . | 0.78 | −0.43 | * | . | F | 2.49 | 0.82 |
| Cys | 55 | . | . | . | . | T | T | . | 0.19 | −0.61 | * | . | F | 3.10 | 0.83 |
| Thr | 56 | . | A | B | . | . | . | . | 0.11 | −0.54 | . | . | F | 1.99 | 0.50 |
| Glu | 57 | . | A | B | . | . | . | . | −0.21 | 0.14 | . | . | . | 0.63 | 0.22 |
| Ala | 58 | . | A | B | . | . | . | . | −0.21 | 0.31 | . | . | . | 0.32 | 0.22 |
| Ala | 59 | . | A | B | . | . | . | . | −0.42 | −0.26 | . | . | . | 0.61 | 0.25 |
| Ile | 60 | . | A | B | . | . | . | . | 0.21 | −0.31 | * | . | . | 0.58 | 0.23 |
| Cys | 61 | . | A | B | . | . | . | . | 0.63 | 0.19 | * | . | . | 0.26 | 0.31 |
| Asp | 62 | . | . | B | . | . | T | . | 0.29 | −0.31 | * | . | . | 1.54 | 0.59 |
| Pro | 63 | . | . | . | . | T | T | . | 0.07 | −0.39 | * | . | F | 2.37 | 0.84 |
| His | 64 | . | . | . | . | T | T | . | 0.41 | −0.39 | * | . | F | 2.80 | 1.29 |
| Arg | 65 | . | . | . | . | T | T | . | 0.63 | −0.20 | * | . | F | 2.52 | 1.21 |
| Gly | 66 | . | . | . | . | T | . | . | 1.30 | 0.37 | * | . | . | 1.14 | 0.42 |
| Leu | 67 | . | . | B | . | . | . | . | 1.06 | −0.06 | * | . | . | 1.06 | 0.51 |
| Tyr | 68 | . | . | B | . | . | . | . | 0.97 | 0.20 | * | . | . | 0.52 | 0.41 |
| Cys | 69 | . | . | B | . | . | T | . | 0.66 | 0.59 | . | . | . | 0.48 | 0.56 |
| Asp | 70 | . | . | B | . | . | T | . | 0.54 | 0.59 | * | . | . | 0.82 | 0.67 |
| Tyr | 71 | . | . | . | . | T | T | . | 1.00 | −0.10 | . | . | . | 2.46 | 0.71 |
| Ser | 72 | . | . | . | . | T | T | . | 1.60 | −0.86 | . | * | F | 3.40 | 2.60 |
| Gly | 73 | . | . | . | . | T | . | . | 1.96 | −1.00 | . | * | F | 2.86 | 2.41 |
| Asp | 74 | . | . | . | . | T | . | . | 2.38 | −1.00 | * | * | F | 2.66 | 3.01 |
| Arg | 75 | . | . | B | . | . | T | . | 1.79 | −1.00 | * | * | F | 2.26 | 3.52 |
| Pro | 76 | . | . | . | . | T | T | . | 1.14 | −0.89 | . | * | F | 2.46 | 3.59 |
| Arg | 77 | . | . | . | . | T | T | . | 1.10 | −0.63 | . | * | . | 2.11 | 1.51 |
| Tyr | 78 | . | . | B | . | . | T | . | 0.59 | −0.20 | . | * | . | 1.40 | 0.76 |
| Ala | 79 | . | . | B | B | . | . | . | −0.08 | 0.44 | . | * | . | −0.04 | 0.37 |
| Ile | 80 | . | . | B | B | . | . | . | −0.78 | 0.59 | . | * | . | −0.18 | 0.10 |
| Gly | 81 | . | . | B | B | . | . | . | −0.57 | 1.09 | * | * | . | −0.32 | 0.06 |
| Val | 82 | . | . | B | B | . | . | . | −1.53 | 0.73 | * | * | . | −0.46 | 0.11 |
| Cys | 83 | . | . | B | B | . | . | . | −2.14 | 0.87 | . | * | . | −0.60 | 0.12 |
| Ala | 84 | . | . | B | B | . | . | . | −1.90 | 0.83 | . | . | . | −0.60 | 0.09 |
| Gln | 85 | . | . | B | B | . | . | . | −1.87 | 0.83 | . | * | . | −0.60 | 0.12 |
| Val | 86 | . | . | B | B | . | . | . | −1.87 | 0.83 | . | . | . | −0.60 | 0.16 |
| Val | 87 | . | . | B | B | . | . | . | −1.68 | 0.69 | . | . | . | −0.60 | 0.16 |
| Gly | 88 | . | . | B | . | . | T | . | −1.87 | 0.76 | . | . | . | −0.20 | 0.05 |
| Val | 89 | . | . | B | . | . | T | . | −2.09 | 1.00 | . | . | . | −0.20 | 0.05 |
| Gly | 90 | . | . | B | . | . | T | . | −2.09 | 1.04 | . | . | . | −0.20 | 0.05 |
| Cys | 91 | . | . | B | . | . | T | . | −1.58 | 0.40 | * | . | . | 0.10 | 0.09 |
| Val | 92 | . | . | B | B | . | . | . | −1.58 | 0.40 | * | . | . | −0.30 | 0.12 |
| Leu | 93 | . | . | B | B | . | . | . | −1.12 | 0.40 | * | . | . | −0.30 | 0.09 |
| Asp | 94 | . | . | B | B | . | . | . | −0.51 | −0.03 | * | * | . | 0.30 | 0.34 |
| Gly | 95 | . | . | B | B | . | . | . | −0.17 | 0.16 | * | . | . | −0.30 | 0.71 |
| Val | 96 | . | . | B | B | . | . | . | 0.50 | −0.09 | * | . | . | 0.45 | 1.39 |
| Arg | 97 | . | . | B | B | . | . | . | 1.01 | −0.37 | * | . | . | 0.45 | 1.34 |
| Tyr | 98 | . | . | . | B | T | . | . | 1.82 | 0.06 | . | . | . | 0.25 | 1.34 |
| Asn | 99 | . | . | . | . | T | T | . | 1.52 | 0.03 | . | . | F | 0.80 | 3.12 |
| Asn | 100 | . | . | . | . | T | T | . | 1.17 | −0.23 | . | * | F | 1.40 | 2.13 |
| Gly | 101 | . | . | . | . | T | T | . | 2.02 | 0.56 | . | * | F | 0.50 | 1.18 |
| Gln | 102 | . | . | . | . | T | T | . | 1.70 | 0.20 | . | * | F | 0.80 | 1.27 |
| Ser | 103 | . | . | . | . | T | . | . | 1.94 | 0.23 | . | . | F | 0.60 | 1.22 |
| Phe | 104 | . | . | B | . | . | . | . | 1.28 | 0.23 | * | * | F | 0.48 | 1.98 |
| Gln | 105 | . | . | B | . | . | T | . | 1.32 | 0.37 | . | * | F | 0.81 | 0.61 |
| Pro | 106 | . | . | . | . | T | T | . | 1.42 | −0.03 | . | * | F | 2.09 | 0.92 |
| Asn | 107 | . | . | . | . | T | T | . | 1.42 | 0.34 | . | * | F | 1.92 | 1.66 |
| Cys | 108 | . | . | . | . | T | T | . | 1.06 | −0.04 | . | * | F | 2.80 | 1.54 |
| Lys | 109 | . | . | . | . | T | T | . | 1.44 | 0.13 | . | * | . | 1.62 | 0.53 |
| Tyr | 110 | . | . | . | . | T | T | . | 0.78 | 0.19 | . | * | . | 1.34 | 0.48 |
| Asn | 111 | . | . | . | . | T | T | . | 0.10 | 0.36 | . | * | . | 1.06 | 0.48 |
| Cys | 112 | . | . | B | . | . | T | . | 0.10 | 0.47 | . | * | . | 0.08 | 0.17 |
| Thr | 113 | . | . | B | . | . | . | . | 0.42 | 0.47 | * | . | . | −0.40 | 0.18 |
| Cys | 114 | . | . | B | . | . | T | . | −0.21 | 0.14 | * | * | . | 0.10 | 0.11 |
| Ile | 115 | . | . | B | . | . | T | . | −0.82 | 0.24 | . | . | . | 0.10 | 0.21 |
| Asp | 116 | . | . | B | . | . | T | . | −1.17 | 0.31 | . | . | . | 0.10 | 0.11 |
| Gly | 117 | . | . | B | . | . | T | . | −1.17 | 0.26 | . | . | . | 0.10 | 0.20 |
| Ala | 118 | . | . | . | B | T | . | . | −1.17 | 0.26 | . | . | . | 0.10 | 0.15 |
| Val | 119 | . | . | B | B | . | . | . | −0.71 | 0.06 | . | . | . | −0.30 | 0.13 |
| Gly | 120 | . | . | . | B | T | . | . | −0.63 | 0.49 | . | . | . | −0.20 | 0.20 |
| Cys | 121 | . | . | B | B | . | . | . | −1.30 | 0.74 | . | . | . | −0.60 | 0.17 |
| Thr | 122 | . | . | B | . | . | T | . | −1.77 | 0.81 | . | * | . | −0.20 | 0.12 |
| Pro | 123 | . | . | B | . | . | T | . | −1.07 | 0.86 | . | * | F | −0.05 | 0.10 |
| Leu | 124 | . | . | B | . | . | T | . | −1.07 | 0.43 | . | * | . | −0.20 | 0.37 |
| Cys | 125 | . | . | B | . | . | T | . | −0.61 | 0.50 | . | * | . | −0.20 | 0.19 |
| Leu | 126 | . | . | B | B | . | . | . | −0.16 | 0.01 | . | * | . | −0.30 | 0.24 |
| Arg | 127 | . | . | B | B | . | . | . | −0.06 | 0.01 | . | * | . | −0.30 | 0.45 |
| Val | 128 | . | . | B | B | . | . | . | 0.27 | −0.24 | . | * | . | 0.45 | 1.29 |
| Arg | 129 | . | . | B | B | . | . | . | 0.27 | −0.81 | . | * | F | 0.90 | 3.06 |
| Pro | 130 | . | . | B | . | . | T | . | 0.64 | −0.81 | . | * | F | 1.30 | 1.29 |
| Pro | 131 | . | . | . | . | T | T | . | 0.79 | 0.10 | . | * | F | 0.80 | 1.82 |
| Arg | 132 | . | . | . | . | T | T | . | 0.47 | 0.03 | . | * | . | 0.50 | 0.50 |
| Leu | 133 | . | . | . | . | T | T | . | 1.29 | 0.46 | * | . | . | 0.20 | 0.50 |
| Trp | 134 | . | . | B | . | . | . | . | 0.97 | 0.53 | * | . | . | −0.12 | 0.44 |
| Cys | 135 | . | . | B | . | . | . | . | 1.29 | 0.53 | * | . | . | 0.16 | 0.35 |
| Pro | 136 | . | . | B | . | . | . | . | 1.61 | 0.53 | * | . | . | 0.44 | 0.82 |
| His | 137 | . | . | . | . | . | T | C | 0.64 | −0.16 | * | . | . | 2.17 | 1.53 |
| Pro | 138 | . | . | . | . | T | T | . | 1.16 | −0.43 | * | . | F | 2.80 | 2.12 |
| Arg | 139 | . | . | . | . | T | T | . | 0.56 | −0.61 | * | . | F | 2.82 | 1.84 |
| Arg | 140 | . | . | . | . | T | T | . | 1.01 | −0.36 | * | . | F | 2.09 | 0.95 |
| Val | 141 | . | . | . | . | T | . | . | 0.88 | −0.43 | * | * | F | 1.61 | 0.95 |
| Ser | 142 | . | . | . | . | T | . | . | 0.88 | −0.43 | * | * | . | 1.18 | 0.48 |
| Ile | 143 | . | . | B | . | . | T | . | 0.42 | 0.07 | * | * | F | 0.25 | 0.33 |
| Pro | 144 | . | . | . | . | T | T | . | −0.36 | 0.64 | * | * | F | 0.35 | 0.24 |
| Gly | 145 | . | . | . | . | T | T | . | −0.47 | 0.57 | . | * | . | 0.20 | 0.10 |
| His | 146 | . | . | . | . | T | T | . | 0.39 | 0.19 | * | * | . | 0.50 | 0.24 |
| Cys | 147 | . | A | . | . | T | . | . | 0.40 | −0.10 | . | . | . | 0.70 | 0.27 |
| Cys | 148 | . | A | . | . | T | . | . | 0.43 | 0.39 | . | . | . | 0.10 | 0.28 |
| Glu | 149 | . | A | . | . | T | . | . | −0.02 | 0.60 | * | . | . | −0.20 | 0.15 |
| Gln | 150 | . | A | . | . | T | . | . | 0.32 | 0.67 | . | . | . | −0.20 | 0.15 |
| Trp | 151 | . | A | B | . | . | . | . | 0.36 | 0.10 | . | . | . | 0.04 | 0.50 |
| Val | 152 | . | A | B | . | . | . | . | 1.02 | −0.47 | . | . | . | 0.98 | 0.48 |
| Cys | 153 | . | . | B | . | . | T | . | 1.10 | −0.47 | . | . | . | 1.72 | 0.46 |
| Glu | 154 | . | . | . | . | T | T | . | 1.14 | −0.37 | . | . | . | 2.46 | 0.45 |
| Asp | 155 | . | . | . | . | T | T | . | 1.26 | −1.29 | . | . | F | 3.40 | 1.20 |
| Asp | 156 | . | . | . | . | T | T | . | 1.33 | −1.93 | . | * | F | 3.06 | 4.38 |
| Ala | 157 | . | . | . | . | T | . | . | 2.30 | −2.07 | * | . | F | 2.86 | 3.91 |
| Lys | 158 | . | . | . | . | T | . | . | 3.01 | −2.07 | * | . | F | 2.86 | 4.59 |
| Arg | 159 | . | . | . | . | . | T | C | 2.70 | −2.07 | * | . | F | 2.86 | 5.49 |
| Pro | 160 | . | . | . | . | T | T | . | 2.11 | −1.59 | * | . | F | 3.06 | 7.85 |
| Arg | 161 | . | . | . | . | T | T | . | 1.90 | −1.59 | * | . | F | 3.40 | 3.96 |
| Lys | 162 | . | . | . | . | T | T | . | 2.60 | −1.16 | * | . | F | 3.06 | 3.13 |
| Thr | 163 | . | . | B | . | . | . | . | 2.56 | −1.16 | * | . | F | 2.12 | 3.96 |
| Ala | 164 | . | . | B | . | . | . | . | 2.13 | −1.59 | * | * | F | 1.78 | 3.38 |
| Pro | 165 | . | . | B | . | . | . | . | 2.00 | −1.10 | . | . | F | 1.71 | 2.44 |
| Arg | 166 | . | . | B | . | . | . | . | 1.30 | −0.67 | * | . | F | 1.64 | 1.67 |
| Asp | 167 | . | . | B | . | . | T | . | 0.56 | −0.66 | * | * | F | 2.11 | 1.67 |
| Thr | 168 | . | . | B | . | . | T | . | 0.87 | −0.37 | . | * | F | 1.93 | 0.94 |
| Gly | 169 | . | . | . | . | . | T | C | 0.87 | −0.80 | . | . | F | 2.70 | 0.80 |
| Ala | 170 | . | . | B | . | . | T | . | 0.22 | −0.30 | . | * | . | 1.78 | 0.48 |
| Phe | 171 | . | A | B | . | . | . | . | −0.23 | 0.34 | * | * | . | 0.51 | 0.25 |
| Asp | 172 | . | A | B | . | . | . | . | −0.23 | 0.29 | * | * | . | 0.24 | 0.25 |
| Ala | 173 | . | A | B | . | . | . | . | −0.78 | −0.14 | * | * | . | 0.57 | 0.43 |
| Val | 174 | . | A | B | . | . | . | . | −0.43 | 0.00 | * | * | . | −0.30 | 0.37 |
| Gly | 175 | . | A | . | . | . | . | C | −0.43 | −0.79 | * | * | . | 0.80 | 0.38 |
| Glu | 176 | A | A | . | . | . | . | . | −0.02 | −0.29 | . | * | . | 0.30 | 0.38 |
| Val | 177 | A | A | . | . | . | . | . | −0.06 | 0.13 | * | * | . | −0.30 | 0.54 |
| Glu | 178 | A | A | . | . | . | . | . | 0.64 | −0.01 | * | * | . | 0.30 | 0.74 |
| Ala | 179 | A | A | . | . | . | . | . | 1.50 | −0.44 | * | . | . | 0.30 | 0.83 |
| Trp | 180 | A | A | . | . | . | . | . | 1.18 | −0.04 | * | * | . | 0.45 | 1.81 |
| His | 181 | A | . | . | . | . | T | . | 0.29 | −0.11 | * | * | . | 0.70 | 0.56 |
| Arg | 182 | . | . | . | . | T | T | . | 0.56 | 0.57 | * | * | . | 0.20 | 0.39 |
| Asn | 183 | . | . | . | . | T | T | . | 0.31 | 0.57 | . | . | . | 0.20 | 0.37 |
| Cys | 184 | . | . | . | . | T | T | . | 0.59 | 0.41 | . | . | . | 0.20 | 0.43 |
| Ile | 185 | . | . | . | . | T | . | . | 0.58 | 0.40 | . | . | . | 0.00 | 0.32 |
| Ala | 186 | . | . | . | . | T | . | . | 0.40 | 0.79 | . | . | . | 0.00 | 0.26 |
| Tyr | 187 | . | . | . | . | T | T | . | 0.00 | 0.81 | * | . | . | 0.20 | 0.76 |
| Thr | 188 | . | . | . | . | T | T | . | −0.30 | 1.16 | . | . | F | 0.50 | 1.14 |
| Ser | 189 | . | . | . | . | . | T | C | 0.16 | 0.86 | . | . | F | 0.30 | 1.51 |
| Pro | 190 | . | . | . | . | T | T | . | 0.38 | 0.79 | . | . | F | 0.50 | 1.49 |
| Trp | 191 | . | . | . | . | T | . | . | 0.67 | 0.60 | . | . | F | 0.15 | 0.55 |
| Ser | 192 | . | . | . | . | . | T | C | 0.60 | 0.50 | . | . | F | 0.15 | 0.55 |
| Pro | 193 | . | . | . | . | T | T | . | 0.61 | 0.60 | . | . | F | 0.35 | 0.52 |
| Cys | 194 | . | . | . | . | T | T | . | 0.24 | 0.56 | . | . | F | 0.35 | 0.66 |
| Ser | 195 | . | . | . | . | T | T | . | 0.11 | 0.21 | . | . | F | 0.65 | 0.26 |
| Thr | 196 | . | . | . | . | T | T | . | −0.41 | 0.26 | . | . | F | 0.65 | 0.17 |
| Ser | 197 | . | . | . | . | T | T | . | −0.46 | 0.51 | . | . | F | 0.35 | 0.26 |
| Cys | 198 | . | . | B | . | . | T | . | −1.10 | 0.37 | . | . | . | 0.10 | 0.19 |
| Gly | 199 | . | . | . | . | T | T | . | −0.73 | 0.63 | . | . | . | 0.20 | 0.10 |
| Leu | 200 | . | . | B | B | . | . | . | −0.74 | 0.53 | . | * | . | −0.60 | 0.10 |
| Gly | 201 | . | . | B | B | . | . | . | −0.32 | 0.63 | * | * | . | −0.60 | 0.27 |
| Val | 202 | . | . | B | B | . | . | . | −0.91 | 0.06 | * | * | . | −0.30 | 0.53 |
| Ser | 203 | . | . | B | B | . | . | . | −0.54 | 0.31 | * | * | F | −0.15 | 0.45 |
| Thr | 204 | . | . | B | B | . | . | . | −0.20 | 0.01 | * | * | F | −0.15 | 0.61 |
| Arg | 205 | . | . | B | B | . | . | . | −0.24 | −0.01 | . | * | F | 0.60 | 1.31 |
| Ile | 206 | . | . | B | B | . | . | . | 0.10 | −0.01 | . | * | F | 0.45 | 0.73 |
| Ser | 207 | . | . | B | . | . | . | . | 0.37 | 0.00 | * | * | F | 0.05 | 0.81 |
| Asn | 208 | . | . | . | . | T | T | . | 0.67 | 0.01 | * | * | F | 0.65 | 0.42 |
| Val | 209 | . | . | . | . | T | T | . | 0.31 | 0.41 | * | . | . | 0.35 | 1.03 |
| Asn | 210 | . | . | . | . | T | T | . | −0.09 | 0.30 | * | . | . | 0.50 | 0.41 |
| Ala | 211 | . | . | . | . | T | T | . | 0.59 | 0.83 | . | * | . | 0.20 | 0.27 |
| Gln | 212 | . | . | . | . | T | . | . | 0.89 | 0.86 | . | * | . | 0.00 | 0.56 |
| Cys | 213 | . | . | . | . | T | . | . | 0.89 | 0.21 | . | * | . | 0.64 | 0.61 |
| Trp | 214 | . | . | B | . | . | T | . | 1.74 | 0.21 | . | * | . | 0.93 | 1.04 |
| Pro | 215 | . | . | . | . | . | T | C | 1.44 | −0.29 | * | * | F | 2.22 | 1.04 |
| Glu | 216 | . | . | . | . | T | T | . | 2.14 | −0.30 | * | * | F | 2.76 | 2.59 |
| Gln | 217 | . | . | . | . | T | T | . | 1.33 | −0.87 | * | * | F | 3.40 | 4.83 |
| Glu | 218 | . | . | . | . | T | . | . | 1.33 | −1.10 | * | . | F | 2.86 | 2.58 |
| Ser | 219 | . | . | . | . | T | . | . | 1.62 | −0.96 | * | . | F | 2.37 | 0.80 |
| Arg | 220 | . | . | . | . | T | . | . | 1.02 | −0.56 | . | * | F | 2.03 | 0.74 |
| Leu | 221 | . | . | . | . | T | . | . | 1.13 | −0.27 | . | * | . | 1.24 | 0.35 |
| Cys | 222 | . | . | . | . | T | . | . | 0.92 | −0.27 | . | . | . | 0.90 | 0.52 |
| Asn | 223 | . | . | . | . | T | . | . | 0.26 | −0.23 | . | . | . | 1.18 | 0.41 |
| Leu | 224 | . | . | . | . | T | . | . | 0.56 | 0.34 | . | . | . | 0.86 | 0.26 |
| Arg | 225 | . | . | B | . | . | T | . | −0.41 | −0.34 | . | . | . | 1.54 | 0.82 |
| Pro | 226 | . | . | . | . | T | T | . | 0.40 | −0.27 | . | * | F | 2.37 | 0.38 |
| Cys | 227 | . | . | . | . | T | T | . | 0.18 | −0.67 | . | * | . | 2.80 | 0.77 |
| Asp | 228 | . | . | B | . | . | T | . | 0.14 | −0.67 | . | * | . | 2.12 | 0.28 |
| Val | 229 | . | . | B | B | . | . | . | 0.64 | −0.17 | * | . | . | 1.14 | 0.24 |
| Asp | 230 | . | . | B | B | . | . | . | −0.28 | −0.11 | * | * | . | 0.86 | 0.65 |
| Ile | 231 | . | . | B | B | . | . | . | −0.96 | 0.00 | * | * | . | −0.02 | 0.32 |
| His | 232 | . | . | B | B | . | . | . | −0.24 | 0.69 | * | * | . | −0.60 | 0.30 |
| Thr | 233 | . | . | B | B | . | . | . | −0.83 | 0.04 | * | * | . | −0.30 | 0.36 |
| Leu | 234 | . | . | B | B | . | . | . | −0.32 | 0.54 | * | * | . | −0.29 | 0.53 |
| Ile | 235 | . | . | B | B | . | . | . | −0.28 | 0.29 | * | * | . | 0.32 | 0.38 |
| Lys | 236 | . | . | . | B | T | . | . | 0.66 | −0.21 | * | * | F | 1.78 | 0.53 |
| Ala | 237 | . | . | . | . | T | . | . | 0.02 | −0.70 | * | . | F | 2.74 | 1.28 |
| Gly | 238 | . | . | . | . | T | T | . | −0.48 | −0.81 | * | . | F | 3.10 | 0.98 |
| Lys | 239 | . | . | . | . | T | T | . | −0.26 | −0.81 | * | . | F | 2.79 | 0.41 |
| Lys | 240 | . | . | B | . | . | T | . | −0.22 | −0.31 | * | . | F | 1.78 | 0.41 |
| Cys | 241 | . | . | B | . | . | T | . | −0.51 | −0.17 | * | . | . | 1.32 | 0.30 |
| Leu | 242 | . | A | B | B | . | . | . | 0.08 | 0.16 | * | . | . | 0.01 | 0.24 |
| Ala | 243 | . | A | B | B | . | . | . | 0.21 | 0.56 | . | . | . | −0.60 | 0.21 |
| Val | 244 | . | A | B | B | . | . | . | 0.17 | 0.99 | . | . | . | −0.60 | 0.59 |
| Tyr | 245 | . | A | B | . | . | . | . | −0.47 | 0.41 | . | . | . | −0.45 | 1.25 |
| Gln | 246 | . | A | B | . | . | . | . | −0.10 | 0.23 | . | . | . | −0.15 | 1.25 |
| Pro | 247 | . | A | B | . | . | . | . | 0.11 | 0.11 | . | * | F | 0.00 | 2.26 |
| Glu | 248 | . | A | . | . | T | . | . | 0.70 | 0.09 | . | * | F | 0.40 | 1.42 |
| Ala | 249 | . | A | B | . | . | . | . | 0.86 | −0.27 | . | * | F | 0.60 | 1.32 |
| Ser | 250 | . | . | B | . | . | T | . | 0.79 | 0.11 | . | * | . | 0.10 | 0.74 |
| Met | 251 | . | . | B | . | . | T | . | −0.02 | 0.17 | . | * | . | 0.10 | 0.62 |
| Asn | 252 | . | . | B | . | . | T | . | −0.40 | 0.86 | . | * | . | −0.20 | 0.50 |
| Phe | 253 | . | . | B | . | . | T | . | −0.74 | 0.86 | . | * | . | −0.20 | 0.38 |
| Thr | 254 | . | . | B | B | . | . | . | −0.82 | 0.90 | . | * | . | −0.60 | 0.38 |
| Leu | 255 | . | . | B | B | . | . | . | −1.41 | 0.86 | . | * | . | −0.60 | 0.13 |
| Ala | 256 | . | . | B | B | . | . | . | −1.11 | 1.14 | . | * | . | −0.60 | 0.10 |
| Gly | 257 | . | . | B | B | . | . | . | −1.42 | 0.74 | . | * | . | −0.60 | 0.10 |
| Cys | 258 | . | . | B | B | . | . | . | −0.61 | 0.74 | . | * | . | −0.60 | 0.17 |
| Ile | 259 | . | . | B | B | . | . | . | −0.60 | 0.06 | . | * | . | −0.30 | 0.32 |
| Ser | 260 | . | . | B | . | . | T | . | −0.03 | −0.06 | . | * | . | 1.00 | 0.44 |
| Thr | 261 | . | . | B | . | . | T | . | 0.56 | 0.27 | * | . | F | 1.00 | 1.28 |
| Arg | 262 | . | . | B | . | . | T | . | 0.69 | 0.10 | . | * | F | 1.30 | 3.16 |
| Ser | 263 | . | . | . | . | T | T | . | 1.40 | −0.16 | . | . | F | 2.60 | 3.64 |
| Tyr | 264 | . | . | . | . | T | . | . | 2.04 | −0.54 | . | . | F | 3.00 | 5.05 |
| Gln | 265 | . | . | . | . | T | T | . | 1.68 | −0.27 | . | . | F | 2.60 | 4.04 |
| Pro | 266 | . | . | . | . | T | T | . | 1.64 | 0.30 | . | . | F | 1.70 | 1.62 |
| Lys | 267 | . | . | . | . | T | T | . | 0.68 | 0.34 | . | . | . | 1.25 | 1.02 |
| Tyr | 268 | . | . | . | . | T | T | . | 0.31 | 0.23 | . | . | . | 0.80 | 0.44 |
| Cys | 269 | . | . | B | B | . | . | . | −0.04 | 0.40 | . | . | . | −0.60 | 0.15 |
| Gly | 270 | . | . | B | B | . | . | . | −0.04 | 0.59 | . | . | . | −0.60 | 0.08 |
| Val | 271 | . | . | B | B | . | . | . | 0.17 | 0.59 | . | * | . | −0.60 | 0.08 |
| Cys | 272 | . | . | B | B | . | . | . | 0.23 | 0.23 | . | * | . | −0.30 | 0.24 |
| Met | 273 | . | . | B | B | . | . | . | −0.19 | −0.34 | . | * | . | 0.30 | 0.47 |
| Asp | 274 | . | . | . | B | T | . | . | −0.19 | −0.20 | . | * | F | 0.85 | 0.34 |
| Asn | 275 | . | . | . | . | T | T | . | −0.73 | −0.27 | . | * | F | 1.25 | 0.34 |
| Arg | 276 | . | . | . | . | T | T | . | −0.09 | −0.16 | . | * | . | 1.10 | 0.24 |
| Cys | 277 | . | . | B | . | . | T | . | 0.33 | −0.34 | . | * | . | 0.98 | 0.22 |
| Cys | 278 | . | . | B | . | . | T | . | 0.98 | 0.41 | . | * | . | 0.36 | 0.22 |
| Ile | 279 | . | . | B | . | . | . | . | 0.68 | 0.01 | . | * | . | 0.74 | 0.22 |
| Pro | 280 | . | . | B | . | . | . | . | 0.72 | 0.40 | . | * | . | 0.72 | 0.56 |
| Tyr | 281 | . | . | . | . | T | T | . | 0.30 | −0.17 | * | * | F | 2.80 | 2.09 |
| Lys | 282 | . | . | . | . | T | T | . | 0.08 | −0.26 | . | * | F | 2.52 | 4.30 |
| Ser | 283 | . | . | B | . | . | T | . | 0.74 | −0.26 | . | * | F | 1.84 | 1.95 |
| Lys | 284 | . | . | B | . | . | T | . | 0.78 | −0.69 | . | * | F | 1.86 | 2.08 |
| Thr | 285 | . | . | B | B | . | . | . | 0.69 | −0.80 | . | * | F | 1.03 | 0.77 |
| Ile | 286 | . | . | B | B | . | . | . | 0.23 | −0.41 | . | * | F | 0.45 | 0.77 |
| Asp | 287 | . | . | B | B | . | . | . | 0.19 | −0.01 | . | * | . | 0.30 | 0.33 |
| Val | 288 | . | . | B | B | . | . | . | −0.18 | 0.39 | * | * | . | −0.30 | 0.40 |
| Ser | 289 | . | . | B | B | . | . | . | −0.43 | 0.47 | * | * | . | −0.60 | 0.31 |
| Phe | 290 | . | . | B | B | . | . | . | −0.12 | 0.21 | . | * | . | −0.30 | 0.28 |
| Gln | 291 | . | . | B | B | . | . | . | 0.42 | 0.21 | * | * | . | −0.30 | 0.64 |
| Cys | 292 | . | . | B | . | . | T | . | −0.39 | 0.00 | * | * | . | 0.10 | 0.47 |
| Pro | 293 | . | . | . | . | T | T | . | 0.12 | 0.30 | . | * | F | 0.79 | 0.45 |
| Asp | 294 | . | . | . | . | T | T | . | −0.28 | −0.06 | . | . | F | 1.53 | 0.26 |
| Gly | 295 | . | . | . | . | T | T | . | 0.12 | 0.33 | . | . | F | 1.07 | 0.41 |
| Leu | 296 | . | . | . | . | . | . | C | 0.23 | 0.14 | . | . | . | 0.66 | 0.36 |
| Gly | 297 | . | . | . | . | . | . | C | 0.90 | −0.29 | * | . | . | 1.40 | 0.42 |
| Phe | 298 | . | . | B | B | . | . | . | 0.26 | 0.11 | * | . | . | 0.26 | 0.74 |
| Ser | 299 | . | . | B | B | . | . | . | −0.56 | 0.33 | * | . | . | 0.12 | 0.66 |
| Arg | 300 | . | . | B | B | . | . | . | −0.50 | 0.33 | * | . | . | −0.02 | 0.55 |
| Gln | 301 | . | . | B | B | . | . | . | −0.58 | 0.81 | * | . | . | −0.46 | 0.67 |
| Val | 302 | . | . | B | B | . | . | . | −0.23 | 0.71 | * | . | . | −0.60 | 0.35 |
| Leu | 303 | . | . | B | B | . | . | . | −0.12 | 0.73 | * | . | . | −0.60 | 0.29 |
| Trp | 304 | . | . | B | B | . | . | . | −0.49 | 1.23 | * | * | . | −0.60 | 0.17 |
| Ile | 305 | . | . | B | B | . | . | . | −1.30 | 1.40 | * | * | . | −0.60 | 0.12 |
| Asn | 306 | . | . | B | B | . | . | . | −1.97 | 1.54 | . | . | . | −0.60 | 0.13 |
| Ala | 307 | . | . | . | B | T | . | . | −1.11 | 1.43 | . | . | . | −0.20 | 0.06 |
| Cys | 308 | . | . | . | B | T | . | . | −1.11 | 0.91 | . | * | . | −0.20 | 0.15 |
| Phe | 309 | . | . | . | B | T | . | . | −1.12 | 0.91 | * | . | . | −0.20 | 0.08 |
| Cys | 310 | . | . | . | B | T | . | . | −0.90 | 0.90 | * | * | . | −0.20 | 0.10 |
| Asn | 311 | . | . | . | B | T | . | . | −0.79 | 0.97 | . | * | . | −0.20 | 0.10 |
| Leu | 312 | . | . | . | . | T | T | . | −0.20 | 0.40 | * | * | . | 0.20 | 0.23 |
| Ser | 313 | . | . | . | . | T | T | . | 0.26 | 0.01 | * | . | . | 0.84 | 0.69 |
| Cys | 314 | . | . | . | . | T | T | . | 0.96 | −0.13 | * | * | . | 1.78 | 0.66 |
| Arg | 315 | . | . | . | . | T | T | . | 1.62 | −0.13 | * | * | F | 2.42 | 1.29 |
| Asn | 316 | . | . | . | . | . | T | C | 0.73 | −0.81 | * | * | F | 2.86 | 1.61 |
| Pro | 317 | . | . | . | . | T | T | . | 0.84 | −0.51 | * | * | F | 3.40 | 2.10 |
| Asn | 318 | . | . | . | . | T | T | . | 0.56 | −0.30 | * | * | F | 2.61 | 0.93 |
| Asp | 319 | . | . | B | . | . | T | . | 1.22 | 0.20 | * | * | F | 1.27 | 0.58 |
| Ile | 320 | . | A | B | . | . | . | . | 0.30 | −0.20 | * | * | . | 0.98 | 0.63 |
| Phe | 321 | . | A | B | . | . | . | . | 0.30 | 0.06 | * | * | . | 0.04 | 0.32 |
| Ala | 322 | . | A | B | . | . | . | . | 0.21 | −0.34 | * | * | . | 0.30 | 0.34 |
| Asp | 323 | . | A | B | . | . | . | . | −0.03 | 0.04 | * | * | . | −0.30 | 0.64 |
| Leu | 324 | . | A | B | . | . | . | . | −0.24 | 0.11 | * | * | . | −0.15 | 1.16 |
| Glu | 325 | . | A | . | . | T | . | . | 0.64 | −0.24 | * | * | F | 1.28 | 1.78 |
| Ser | 326 | . | A | . | . | . | . | C | 0.64 | −0.74 | . | * | F | 1.66 | 1.78 |
| Tyr | 327 | . | . | . | . | . | T | C | 0.93 | 0.04 | . | * | F | 1.44 | 1.86 |
| Pro | 328 | . | . | . | . | . | T | C | 0.93 | −0.26 | . | * | F | 2.32 | 1.44 |
| Asp | 329 | . | . | . | . | T | T | . | 0.86 | −0.26 | * | . | F | 2.80 | 1.86 |
| Phe | 330 | . | . | B | . | . | T | . | 0.27 | 0.04 | * | . | F | 1.37 | 0.83 |
| Ser | 331 | . | A | B | . | . | . | . | 0.57 | −0.21 | * | . | F | 1.29 | 0.54 |
| Glu | 332 | . | A | B | . | . | . | . | 0.42 | −0.24 | * | . | . | 0.86 | 0.52 |
| Ile | 333 | . | A | B | . | . | . | . | 0.24 | 0.19 | * | . | . | −0.02 | 0.77 |
| Ala | 334 | . | A | . | . | . | . | C | −0.14 | −0.17 | * | . | . | 0.50 | 0.74 |
| Asn | 335 | . | A | . | . | T | . | . | 0.17 | −0.13 | * | . | . | 0.70 | 0.55 |

Among highly preferred fragments in this regard are those that comprise regions of CTGF-4 that combine several structural features, such as several of the features set out above.

The techniques of gene-shuffling, motif-shuffling, exon-shuffling, and/or codon-shuffling (collectively referred to as “DNA shuffling”) may be employed to modulate the activities of CTGF-4 thereby effectively generating agonists and antagonists of CTGF-4. See generally, U.S. Pat. Nos. 5,605,793, 5,811,238, 5,830,721, 5,834,252, and 5,837,458, and Patten, P. A., et al., Curr. Opinion Biotechnol. 8:724-33 (1997); Harayama, S. Trends Biotechnol. 16(2):76-82 (1998); Hansson, L. O., et al., J. Mol. Biol. 287:265-76 (1999); and Lorenzo, M. M. and Blasco, R. Biotechniques 24(2):308-13 (1998) (each of these patents and publications are hereby incorporated by reference). In one embodiment, alteration of CTGF-4 polynucleotides and corresponding polypeptides may be achieved by DNA shuffling. DNA shuffling involves the assembly of two or more DNA segments into a desired CTGF-4 molecule by homologous, or site-specific, recombination. In another embodiment, CTGF-4 polynucleotides and corresponding polypeptides may be altered by being subjected to random mutagenesis by error-prone PCR, random nucleotide insertion or other methods prior to recombination. In another embodiment, one or more components, motifs, sections, parts, domains, fragments, etc., of CTGF-4 may be recombined with one or more components, motifs, sections, parts, domains, fragments, etc. of one or more heterologous molecules. In preferred embodiments, the heterologous molecules are CTGF, CTGF-2, CTGF-3, Cyr61, Cef10, neuroblastoma overexpressed gene, ELM1, rCop-1, WISP-1, WISP-2, WISP-3, or any other member of the CCN family of proteins (which consists of secreted cysteine-rich proteins with growth regulatory functions). In further preferred embodiments, the heterologous molecule is a growth factor such as, for example, platelet-derived growth factor (PDGF), insulin-like growth factor (IGF-I), transforming growth factor (TGF)-alpha, epidermal growth factor (EGF), fibroblast growth factor (FGF), TGF-beta, bone morphogenetic protein (BMP)-2, BMP-4, BMP-5, BMP-6, BMP-7, activins A and B, decapentaplegic(dpp), 60A, OP-2, dorsalin, growth differentiation factors (GDFs), nodal, MIS, inhibin-alpha, TGF-beta1, TGF-beta2, TGF-beta3, TGF-beta5, and glial-derived neurotrophic factor (GDNF).

Other preferred fragments are biologically active CTGF-4 fragments. Biologically active fragments are those exhibiting activity similar, but not necessarily identical, to an activity of the CTGF-4 polypeptide. The biological activity of the fragments may include an improved desired activity, or a decreased undesirable activity.

Transgenics and “Knock-Outs”

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art.

In specific preferred embodiments, CTGF-4 polynucleotides of the invention may be expressed under the direction of a murine transferrin receptor promoter construct thereby restricting expression to the liver of transgenic animals. In other specific preferred embodiments, CTGF-4 polynucleotides of the invention are expressed under the direction of a murine beta-actin promoter construct thereby effecting ubiquitous expression of the CTGF-4 polynucleotide.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR) and “TaqMAN” real time PCR. Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding-of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic and “knock-out” animals of the invention have uses which include, but are not limited to, animal model systems useful in elaborating the biological function of CTGF-4 polypeptides, studying conditions and/or disorders associated with aberrant CTGF-4 expression, and in screening for compounds effective in ameliorating such conditions and/or disorders.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson, et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Epitopes & Antibodies

In the present invention, “epitopes” refer to CTGF-4 polypeptide fragments having antigenic or immunogenic activity in an animal, especially in a human. A preferred embodiment of the present invention relates to a CTGF-4 polypeptide fragment comprising an epitope, as well as the polynucleotide encoding this fragment. A region of a protein molecule to which an antibody can bind is defined as an “antigenic epitope”. In contrast, an “immunogenic epitope” is defined as a part of a protein that elicits an antibody response (See, for instance, Geysen, et al., Proc. Natl. Acad. Sci. USA 81:3998-4002 (1983)).

Fragments which function as epitopes may be produced by any conventional means (See, e.g., Houghten, R. A., Proc. Natl. Acad. Sci. USA 82:5131-5135 (1985); the topic is further described in U.S. Pat. No. 4,631,211).

In the present invention, antigenic epitopes preferably contain a sequence of at least six, preferably at least seven, more preferably at least nine, and most preferably between about 15 to about 30 amino acids. Antigenic epitopes are useful to raise antibodies, including monoclonal antibodies, that specifically bind the epitope (See, for instance, Wilson, et al., Cell 37:767-778 (1984); Sutcliffe, J. G., et al., Science 219:660-666 (1983)).

Similarly, immunogenic epitopes can be used to induce antibodies according to methods well known in the art (See, for instance, Sutcliffe, et al., supra; Wilson, et al., supra; Chow, M., et al., Proc. Natl. Acad. Sci. USA 82:910-914; and Bittle, F. J., et al., J. Gen. Virol. 66:2347-2354 (1985)). A preferred immunogenic epitope includes the secreted protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Using the Protean component of DNAstar analysis computer software, SEQ ID NO:2 was found antigenic at amino acids: Ala-5 to Cys-17, Cys-21 to Cys-30, Ile-37 to Lys-45, Gln-50 to Glu-57, Asp-62 to Tyr-68, Tyr-71 to Tyr-78, Phe-104 to Asn-111, Val-128 to Leu-133, Pro-136 to Ser-142, Val-152 to Ala-170, Cys-213 to Leu-221, Asn-223 to Asp-230, Ile-235 to Cys-241, Ser-260 to Tyr-268, Met-273 to Cys-278, Tyr-281 to Ile-286, Pro-293 to Ser-299, Leu-312 to Ile-320, and Glu-325 to Glu-332. Thus, these regions can be used as epitopes to produce antibodies against the protein encoded by the cDNA clone encoding CTGF-4 designated HWHGU74.

As used herein, the term “antibody” (Ab) or “monoclonal antibody” (Mab) is meant to include intact molecules as well as antibody fragments (such as, for example, Fab and F(ab′)₂ fragments) which are capable of specifically binding to protein. Fab and F(ab′)₂ fragments lack the Fc fragment of intact antibody, clear more rapidly from the circulation, and may have less non-specific tissue binding than an intact antibody (Wahl, et al., J. Nucl. Med. 24:316-325 (1983)). Thus, these fragments are preferred, as well as the products of a FAB or other immunoglobulin expression library. Moreover, antibodies of the present invention include chimeric, single chain, and humanized antibodies.

The present invention further relates to antibodies and T-cell antigen receptors (TCR) which specifically bind the polypeptides of the present invention. The antibodies of the present invention include IgG (including IgG1, IgG2, IgG3, and IgG4), IgA (including IgA1 and IgA2), IgD, IgE, or IgM, and IgY. As used herein, the term “antibody” (Ab) is meant to include whole antibodies, including single-chain whole antibodies, and antigen-binding fragments thereof. Most preferably the antibodies are human antigen binding antibody fragments of the present invention include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are human, murine, rabbit, goat, guinea pig, camel, horse, or chicken.

Antigen-binding antibody fragments, including single-chain antibodies, may comprise the variable region(s) alone or in combination with the entire or partial of the following: hinge region, CH1, CH2, and CH3 domains. Also included in the invention are any combinations of variable region(s) and hinge region, CH1, CH2, and CH3 domains. The present invention further includes monoclonal, polyclonal, chimeric, humanized, and human monoclonal and polyclonal antibodies which specifically bind the polypeptides of the present invention. The present invention further includes antibodies which are anti-idiotypic to the antibodies of the present invention.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for heterologous compositions, such as a heterologous polypeptide or solid support material. See, e.g., WO 93/17715; WO 92/08802; WO 91/00360; WO 92/05793; Tutt, A. et al. (1991) J. Immunol. 147:60-69; U.S. Pat. Nos. 5,573,920, 4,474,893, 5,601,819, 4,714,681, 4,925,648; Kostelny, S. A. et al. (1992) J. Immunol. 148:1547-1553.

Antibodies of the present invention may be described or specified in terms of the epitope(s) or portion(s) of a polypeptide of the present invention which are recognized or specifically bound by the antibody. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homolog of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. Further included in the present invention are antibodies which only bind polypeptides encoded by polynucleotides which hybridize to a polynucleotide of the present invention under stringent hybridization conditions (as described herein). Antibodies of the present invention may also be described or specified in terms of their binding affinity. Preferred binding affinities include those with a dissociation constant or Kd less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

Antibodies of the present invention have uses that include, but are not limited to, methods known in the art to purify, detect, and target the polypeptides of the present invention including both in vitro and in vivo diagnostic and therapeutic methods. For example, the antibodies have use in immunoassays for qualitatively and quantitatively measuring levels of the polypeptides of the present invention in biological samples. See, e.g., Harlow et al., ANTIBODIES: A LABORATORY MANUAL, (Cold Spring Harbor Laboratory Press, 2nd ed. 1988) (incorporated by reference in the entirety).

The antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

The antibodies of the present invention may be prepared by any suitable method known in the art. For example, a polypeptide of the present invention or an antigenic fragment thereof can be administered to an animal in order to induce the production of sera containing polyclonal antibodies. The term “monoclonal antibody” is not a limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technology.

Hybridoma techniques include those known in the art and taught in Harlow et al., ANTIBODIES: A LABORATORY MANUAL, (Cold Spring Harbor Laboratory Press, 2nd ed. 1988); Hammerling, et al., in: MONOCLONAL ANTIBODIES AND T-CELL HYBRIDOMAS 563-681 (Elsevier, N.Y., 1981) (said references incorporated by reference in their entireties).

The antibodies of the present invention may be prepared by any of a variety of standard methods. For example, cells expressing the CTGF-4 polypeptide or an antigenic fragment thereof can be administered to an animal in order to induce the production of sera containing polyclonal antibodies. In a preferred method, a preparation of CTGF-4 polypeptide is prepared and purified to render it substantially free of natural contaminants. Such a preparation is then introduced into an animal in order to produce polyclonal antisera of greater specific activity.

In the most preferred method, the antibodies of the present invention are monoclonal antibodies (or CTGF-4 polypeptide binding fragments thereof). Such monoclonal antibodies can be prepared using hybridoma technology (Kohler et al., Nature 256:495 (1975); Köhler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., (1981) pp. 563-681). In general, such procedures involve immunizing an animal (preferably a mouse) with an CTGF-4 polypeptide antigen or, more preferably, with an CTGF-4 polypeptide-expressing cell. Suitable cells can be recognized by their capacity to bind anti-CTGF-4 polypeptide antibody. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; however, it is preferable to employ the parent myeloma cell line (SP2O), available from the ATCC, Manassas, Va. After fusion, the resulting hybridoma cells are selectively maintained in HAT medium, and then cloned by limiting dilution as described by Wands, et al. (Gastroenterology 80:225-232 (1981)). The hybridoma cells obtained through such a selection are then assayed to identify clones which secrete antibodies capable of binding the CTGF-4 antigen.

Alternatively, additional antibodies capable of binding to the CTGF-4 polypeptide antigen may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, CTGF-4 polypeptide-specific antibodies are used to immunize an animal, preferably a mouse. The splenocytes of such an animal are then used to produce hybridoma cells, and the hybridoma cells are screened to identify clones which produce an antibody whose ability to bind to the CTGF-4 polypeptide-specific antibody can be blocked by the CTGF-4 antigen. Such antibodies comprise anti-idiotypic antibodies to the CTGF-4 polypeptide-specific antibody and can be used to immunize an animal to induce formation of further CTGF-4 polypeptide-specific antibodies.

Fab and F(ab′)₂ fragments may be produced by proteolytic cleavage, using enzymes such as papain (to produce Fab fragments) or pepsin (to produce F(ab′)₂ fragments).

Alternatively, antibodies of the present invention can be produced through the application of recombinant DNA and phage display technology or through synthetic chemistry using methods known in the art. For example, the antibodies of the present invention can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene VIII protein. Examples of phage display methods that can be used to make the antibodies of the present invention include those disclosed in Brinkman U. et al. (1995) J. Immunol. Methods 182:41-50; Ames, R. S. et al. (1995) J. Immunol. Methods 184:177-186; Kettleborough, C. A. et al. (1994) Eur. J. Immunol. 24:952-958; Persic, L. et al. (1997) Gene 187 9-18; Burton, D. R. et al. (1994) Advances in Immunology 57:191-280; PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743 (said references incorporated by reference in their entireties).

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax, R. L. et al. BioTechniques 12(6):864-869 (1992); and Sawai, H. et al. AJRI 34:26-34 (1995); and Better, M. et al. Science 240:1041-1043 (1988) (said references incorporated by reference in their entireties).

Examples of techniques which can be used to produce single-chain Fvs (scFvs) and antibodies include those described in U.S. Pat. Nos. 4,946,778 and 5,258,498; Huston et al. Methods in Enzymology 203:46-88 (1991); Shu, L. et al. PNAS 90:7995-7999 (1993); and Skerra, A. et al. Science 240:1038-1040 (1988). For some uses, including in vivo use of antibodies in humans and in vitro detection assays, it may be preferable to use chimeric, humanized, or human antibodies. Methods for producing chimeric antibodies are known in the art. See e.g., Morrison, Science 229:1202 (1985); Oi et al., BioTechniques 4:214 (1986); Gillies, S. D. et al., J. Immunol. Methods 125:191-202 (1989); and U.S. Pat. No. 5,807,715. Antibodies can be humanized using a variety of techniques including CDR-grafting (EP 0 239 400; WO 91/09967; U.S. Pat. Nos. 5,530,101; and 5,585,089), veneering or resurfacing (EP 0 592 106; EP 0 519 596; Padlan, E. A., Molecular Immunology 28(4/5):489-498 (1991); Studnicka G. M. et al., Protein Engineering 7(6):805-814 (1994); Roguska M. A. et al., PNAS 91:969-973) (1994), and chain shuffling (U.S. Pat. No. 5,565,332). Human antibodies can be made by a variety of methods known in the art including phage display methods described above. See also, U.S. Pat. Nos. 4,444,887, 4,716,111, 5,545,806, and 5,814,318; and WO 98/46645 (said references incorporated by reference in their entireties).

Further included in the present invention are antibodies recombinantly fused or chemically conjugated (including both covalently and non-covalently conjugations) to a polypeptide of the present invention. The antibodies may be specific for antigens other than polypeptides of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al. supra and WO 93/21232; EP 0 439 095; Naramura, M. et al., Immunol. Lett. 39:91-99 (1994); U.S. Pat. No. 5,474,981; Gillies, S. O. et al. PNAS 89:1428-1432 (1992); Fell, H. P. et al., J. Immunol. 146:2446-2452 (1991) (said references incorporated by reference in their entireties).

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the hinge region, CH1 domain, CH2 domain, and CH3 domain or any combination of whole domains or portions thereof. The polypeptides of the present invention may be fused or conjugated to the above antibody portions to increase the in vivo half life of the polypeptides or for use in immunoassays using methods known in the art. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See e.g., U.S. Pat. Nos. 5,336,603, 5,622,929, 5,359,046, 5,349,053, 5,447,851, 5,112,946; EP 0 307 434, EP 0 367 166; WO 96/04388, WO 91/06570; Ashkenazi, A. et al., PNAS 88:10535-10539 (1991); Zheng, X. X. et al., J. Immunol. 154:5590-5600 (1995); and Vil, H. et al., PNAS 89:11337-11341 (1992) (said references incorporated by reference in their entireties).

The invention further relates to antibodies which act as agonists or antagonists of the polypeptides of the present invention. For example, the present invention includes antibodies which disrupt the receptor/ligand interactions with the polypeptides of the invention either partially or fully. Included are both receptor-specific antibodies and ligand-specific antibodies. Included are receptor-specific antibodies which do not prevent ligand binding but prevent receptor activation. Receptor activation (i.e., signaling) may be determined by techniques described herein or otherwise known in the art. Also included are receptor-specific antibodies which both prevent ligand binding and receptor activation. Likewise, included are neutralizing antibodies which bind the ligand and prevent binding of the ligand to the receptor, as well as antibodies which bind the ligand, thereby preventing receptor activation, but do not prevent the ligand from binding the receptor. Further included are antibodies which activate the receptor. These antibodies may act as agonists for either all or less than all of the biological activities affected by ligand-mediated receptor activation. The antibodies may be specified as agonists or antagonists for biological activities comprising specific activities disclosed herein. The above antibody agonists can be made using methods known in the art. See e.g., WO 96/40281; U.S. Pat. No. 5,811,097; Deng, B. et al., Blood 92(6):1981-1988 (1998); Chen, Z. et al., Cancer Res. 58(16):3668-3678 (1998); Harrop, J. A. et al., J. Immunol. 161(4):1786-1794 (1998); Zhu, Z. et al., Cancer Res. 58(15):3209-3214 (1998); Yoon, D. Y. et al., J. Immunol. 160(7):3170-3179 (1998); Prat, M. et al., J. Cell. Sci. 111 (Pt2):237-247 (1998); Pitard, V. et al., J. Immunol. Methods 205(2):177-190 (1997); Liautard, J. et al., Cytokinde 9(4):233-241 (1997); Carlson, N. G. et al., J. Biol. Chem. 272(17):11295-11301 (1997); Taryman, R. E. et al., Neuron 14(4):755-762 (1995); Muller, Y. A. et al., Structure 6(9):1153-1167 (1998); Bartunek, P. et al., Cytokine 8(1):14-20 (1996) (said references incorporated by reference in their entireties).

As discussed above, antibodies to the CTGF-4 polypeptides of the invention can, in turn, be utilized to generate anti-idiotype antibodies that “mimic” the CTGF-4, using techniques well known to those skilled in the art. (See, e.g., Greenspan & Bona, FASEB J. 7(5):437-444 (1989), and Nissinoff, J. Immunol. 147(8):2429-2438 (1991)). For example, antibodies which bind to CTGF-4 and competitively inhibit the CTGF-4 binding to receptor can be used to generate anti-idiotypes that “mimic” the CTGF-4 binding domain and, as a consequence, bind to and neutralize CTGF-4 and/or its receptor. Such neutralizing anti-idiotypes or Fab fragments of such anti-idiotypes can be used in therapeutic regimens to neutralize CTGF-4 ligands.

Fusion Proteins

Any CTGF-4 polypeptide can be used to generate fusion proteins. For example, the CTGF-4 polypeptide, when fused to a second protein, can be used as an antigenic tag. Antibodies raised against the CTGF-4 polypeptide can be used to indirectly detect the second protein by binding to the CTGF-4. Moreover, because secreted proteins target cellular locations based on trafficking signals, the CTGF-4 polypeptides can be used as a targeting molecule once fused to other proteins.

Examples of domains that can be fused to CTGF-4 polypeptides include not only heterologous signal sequences, but also other heterologous functional regions. The fusion does not necessarily need to be direct, but may occur through linker sequences.

Moreover, fusion proteins may also be engineered to improve characteristics of the CTGF-4 polypeptide. For instance, a region of additional amino acids, particularly charged amino acids, may be added to the N-terminus of the CTGF-4 polypeptide to improve stability and persistence during purification from the host cell or subsequent handling and storage. Also, peptide moieties may be added to the CTGF-4 polypeptide to facilitate purification. Such regions may be removed prior to final preparation of the CTGF-4 polypeptide. The addition of peptide moieties to facilitate handling of polypeptides are familiar and routine techniques in the art.

Moreover, CTGF-4 polypeptides, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins (EP A 394,827; Traunecker, et al., Nature 331:84-86 (1988)). Fusion proteins having disulfide-linked dimeric structures (due to the IgG) can also be more efficient in binding and neutralizing other molecules, than the monomeric secreted protein or protein fragment alone (Fountoulakis, et al., J. Biochem. 270:3958-3964 (1995)).

Similarly, EP-A-0 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and, thus, can result in, for example, improved pharmacokinetic properties (EP-A 0232 262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5 (See, Bennett, D., et al., J. Molecular Recognition 8:52-58 (1995); Johanson, K., et al., J. Biol. Chem. 270:9459-9471 (1995)).

Moreover, the CTGF-4 polypeptides can be fused to marker sequences, such as a peptide which facilitates purification of CTGF-4. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described by Gentz and coworkers (Proc. Natl. Acad. Sci. USA 86:821-824 (1989)), for instance, hexa-histidine provides for convenient purification of the fusion protein. Another peptide tag useful for purification, the “HA” tag, corresponds to an epitope derived from the influenza hemagglutinin protein (Wilson, et al., Cell 37:767 (1984)).

In further preferred embodiments, CTGF-4 polynucleotides of the invention are fused to a polynucleotide encoding a “FLAG” polypeptide. Thus, a CTGF-4-FLAG fusion protein is encompassed by the present invention. The FLAG antigenic polypeptide may be fused to a CTGF-4 polypeptide of the invention at either or both the amino or the carboxy terminus. In preferred embodiments, a CTGF-4-FLAG fusion protein is expressed from a pFLAG-CMV-5a or a pFLAG-CMV-1 expression vector (available from Sigma, St. Louis, Mo., USA). See, Andersson, S., et al., J. Biol. Chem. 264:8222-29 (1989); Thomsen, D. R., et al., Proc. Natl. Acad. Sci. USA, 81:659-63 (1984); and Kozak, M., Nature 308:241 (1984) (each of which is hereby incorporated by reference). In further preferred embodiments, a CTGF-4-FLAG fusion protein is detectable by anti-FLAG monoclonal antibodies (also available from Sigma).

Thus, any of these above fusions can be engineered using the CTGF-4 polynucleotides or the polypeptides.

Vectors, Host Cells, and Protein Production

The present invention also relates to vectors containing the CTGF-4 polynucleotide, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

CTGF-4 polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The CTGF-4 polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pHE4, pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, (see, for example, Davis, et al., Basic Methods In Molecular Biology (1986)). It is specifically contemplated that CTGF-4 polypeptides may in fact be expressed by a host cell lacking a recombinant vector.

CTGF-4 polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

CTGF-4 polypeptides, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the CTGF-4 polypeptides may be glycosylated or may be non-glycosylated. In addition, CTGF-4 polypeptides may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Uses of the CTGF-4 Polynucleotides

The CTGF-4 polynucleotides identified herein can be used in numerous ways as reagents. The following description should be considered exemplary and utilizes known techniques.

There exists an ongoing need to identify new chromosome markers, since few chromosome marking reagents, based on actual sequence data (repeat polymorphisms), are presently available. The chromosomal location of clone HWHGU74 can be mapped. Then, CTGF-4 polynucleotides can be used in linkage analysis as a marker for that specific region of that specific chromosome.

Briefly, sequences can be mapped to chromosomes by preparing PCR primers (preferably 15-25 bp) from the sequences shown in SEQ ID NO:1. Primers can be selected using computer analysis so that primers do not span more than one predicted exon in the genomic DNA. These primers are then used for PCR screening of somatic cell hybrids containing individual human chromosomes. Only those hybrids containing the human CTGF-4 gene corresponding to the SEQ ID NO:1 will yield an amplified fragment.

Similarly, somatic hybrids provide a rapid method of PCR mapping the polynucleotides to particular chromosomes. Three or more clones can be assigned per day using a single thermal cycler. Moreover, sublocalization of the CTGF-4 polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the CTGF-4 polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred (for a review, see Verma, et al., “Human Chromosomes: a Manual of Basic Techniques,” Pergamon Press, New York (1988)).

For chromosome mapping, the CTGF-4 polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease (Disease mapping data are found, for example, in V. McKusick, Mendelian Inheritance in Man (available on line through Johns Hopkins University Welch Medical Library)). Assuming 1 megabase mapping resolution and one gene per 20 kb, a cDNA precisely localized to a chromosomal region associated with the disease could be one of 50-500 potential causative genes.

Thus, once coinheritance is established, differences in the CTGF-4 polynucleotide and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the CTGF-4 polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using CTGF-4 polynucleotides. Any of these alterations (altered expression, chromosomal rearrangement, or mutation) can be used as a diagnostic or prognostic marker.

In addition to the foregoing, a CTGF-4 polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee, et al., Nucl. Acids Res. 6:3073 (1979); Cooney, et al., Science 241:456 (1988); and Dervan, et al., Science 251:1360 (1991)) or to the mRNA itself (antisense—see Okano, J. Neurochem. 56:560 (1991); Oligodeoxy-nucleotides as Antisense Inhibitors of Gene Expression, CRC Press, Boca Raton, Fla. (1988)). Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

The CTGF-4 polynucleotides are also useful for identifying individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The CTGF-4 polynucleotides can be used as additional DNA markers for RFLP.

The CTGF-4 polynucleotides can also be used as an alternative to RFLP, by determining the actual base-by-base DNA sequence of selected portions of an individual's genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, etc., can be amplified using PCR. In one prior art technique, gene sequences amplified from polymorphic loci, such as DQa class II HLA gene, are used in forensic biology to identify individuals (Erlich, H., PCR Technology, Freeman and Co. (1992)). Once these specific polymorphic loci are amplified, they are digested with one or more restriction enzymes, yielding an identifying set of bands on a Southern blot probed with DNA corresponding to the DQa class II HLA gene. Similarly, CTGF-4 polynucleotides can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from CTGF-4 sequences. Panels of such reagents can identify tissue by species and/or by organ type. In a similar fashion, these reagents can be used to screen tissue cultures for contamination.

Because CTGF-4 is found expressed in a number of cells and tissues (predominantly in fetal liver, lymph node, kidney, and ovary, and to lesser extents in other tissues), CTGF-4 polynucleotides are useful as hybridization probes for differential identification of the tissue(s) or cell type(s) present in a biological sample. Similarly, polypeptides and antibodies directed to CTGF-4 polypeptides are useful to provide immunological probes for differential identification of the tissue(s) or cell type(s). In addition, for a number of disorders of the above tissues or cells, particularly of the immune, urinary, digestive, and reproductive systems, significantly higher or lower levels of CTGF-4 gene expression may be detected in certain tissues (e.g., cancerous and wounded tissues) or bodily fluids (e.g., serum, plasma, urine, synovial fluid or spinal fluid) taken from an individual having such a disorder, relative to a “standard” CTGF-4 gene expression level, i.e., the CTGF-4 expression level in healthy tissue from an individual not having the immune, urinary, digestive, and reproductive systems disorder.

Thus, the invention provides a diagnostic method of a disorder, which involves: (a) assaying CTGF-4 gene expression level in cells or body fluid of an individual; (b) comparing the CTGF-4 gene expression level with a standard CTGF-4 gene expression level, whereby an increase or decrease in the assayed CTGF-4 gene expression level compared to the standard expression level is indicative of disorder in the immune, urinary, digestive, and reproductive systems.

In the very least, the CTGF-4 polynucleotides can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

Gene Therapy Methods

CTGF-4 polynucleotides are also useful in gene therapy. One goal of gene therapy is to insert a normal gene into an organism having a defective gene, in an effort to correct the genetic defect. CTGF-4 offers a means of targeting such genetic defects in a highly accurate manner. Another goal is to insert a new gene that was not present in the host genome, thereby producing a new trait in the host cell.

Another embodiment of the present invention is to use gene therapy methods for treating disorders, diseases and conditions. The gene therapy methods relate to the introduction of nucleic acid (DNA, RNA and antisense DNA or RNA) sequences into an animal to achieve expression of the CTGF-4 polypeptide of the present invention. This method requires a polynucleotide which codes for a CTGF-4 polypeptide operatively linked to a promoter and any other genetic elements necessary for the expression of the polypeptide by the target tissue. Such gene therapy and delivery techniques are known in the art, see, for example, WO90/11092, which is herein incorporated by reference.

Thus, for example, cells from a patient may be engineered with a polynucleotide (DNA or RNA) comprising a promoter operably linked to a CGTF-4 polynucleotide ex vivo, with the engineered cells then being provided to a patient to be treated with the polypeptide. Such methods are well-known in the art. For example, see Belldegrun, A., et al., J. Natl. Cancer Inst. 85: 207-216 (1993); Ferrantini, M. et al., Cancer Research 53: 1107-1112 (1993); Ferrantini, M. et al., J. Immunology 153: 4604-4615 (1994); Kaido, T., et al., Int. J. Cancer 60: 221-229 (1995); Ogura, H., et al., Cancer Research 50: 5102-5106 (1990); Santodonato, L., et al., Human Gene Therapy 7:1-10 (1996); Santodonato, L., et al., Gene Therapy 4:1246-1255 (1997); and Zhang, J.-F. et al., Cancer Gene Therapy 3: 31-38 (1996)), which are herein incorporated by reference. In one embodiment, the cells which are engineered are arterial cells. The arterial cells may be reintroduced into the patient through direct injection to the artery, the tissues surrounding the artery, or through catheter injection.

As discussed in more detail below, the CTGF-4 polynucleotide constructs can be delivered by any method that delivers injectable materials to the cells of an animal, such as, injection into the interstitial space of tissues (heart, muscle, skin, lung, liver, and the like). The CTGF-4 polynucleotide constructs may be delivered in a pharmaceutically acceptable liquid or aqueous carrier.

In one embodiment, the CTGF-4 polynucleotide is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, the CTGF-4 polynucleotides can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

The CTGF-4 polynucleotide vector constructs used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF1/V5, pcDNA3.1, and pRc/CMV2 available from Invitrogen. Other suitable vectors will be readily apparent to the skilled artisan.

Any strong promoter known to those skilled in the art can be used for driving the expression of CTGF-4 DNA. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the beta-actin promoter; and human growth hormone promoters. The promoter also may be the native promoter for CTGF-4.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The CTGF-4 polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked acid sequence injection, an effective dosage amount of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration.

The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked CTGF-4 DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art.

As is evidenced in the Examples, naked CTGF-4 nucleic acid sequences can be administered in vivo results in the successful expression of CTGF-4 polypeptide in the femoral arteries of rabbits.

The constructs may also be delivered with delivery vehicles such as viral sequences, viral particles, liposome formulations, lipofectin, precipitating agents, etc. Such methods of delivery are known in the art.

In certain embodiments, the CTGF-4 polynucleotide constructs are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA (1987) 84:7413-7416, which is herein incorporated by reference); mRNA (Malone et al., Proc. Natl. Acad. Sci. USA (1989) 86:6077-6081, which is herein incorporated by reference); and purified transcription factors (Debs et al., J. Biol. Chem. (1990) 265:10189-10192, which is herein incorporated by reference), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl. Acad. Sci. USA (1987) 84:7413-7416, which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication No. WO 90/11092 (which is herein incorporated by reference) for a description of the synthesis of DOTAP (1,2-bis(oleoyloxy)-3-(trimethylammonio)propane) liposomes. Preparation of DOTMA liposomes is explained in the literature, see, e.g., P. Felgner et al., Proc. Natl. Acad. Sci. USA 84:7413-7417, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15° C. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et, al., Methods of Immunology (1983), 101:512-527, which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca²⁺-EDTA chelation (Papahadjopoulos et al., Biochim. Biophys. Acta (1975) 394:483; Wilson et al., Cell (1979) 17:77); ether injection (Deamer, D. and Bangham, A., Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); detergent dialysis (Enoch, H. and Strittmatter, P., Proc. Natl. Acad. Sci. USA (1979) 76:145); and reverse-phase evaporation (REV) (Fraley et al., J. Biol. Chem. (1980) 255:10431; Szoka, F. and Papahadjopoulos, D., Proc. Natl. Acad. Sci. USA (1978) 75:145; Schaefer-Ridder et al., Science (1982) 215:166), which are herein incorporated by reference.

Generally, the ratio of DNA to liposomes will be from about 10:1 to about 1:10. Preferably, the ration will be from about 5:1 to about 1:5. More preferably, the ration will be about 3:1 to about 1:3. Still more preferably, the ratio will be about 1:1.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication no. WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication no. WO 94/9469 (which are herein incorporated by reference) provide methods for delivering DNA-cationic lipid complexes to mammals.

In certain embodiments, cells are be engineered, ex vivo or in vivo, using a retroviral particle containing RNA which comprises a sequence encoding CTGF-4. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, R-2, R-AM, PA12, T19-14×, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include polynucleotide encoding CTGF-4. Such retroviral vector particles then may be employed, to transduce eukaryotic cells, either in vitro or in vivo. The transduced eukaryotic cells will express CTGF-4.

In certain other embodiments, cells are engineered, ex vivo or in vivo, with CTGF-4 polynucleotide contained in an adenovirus vector. Adenovirus can be manipulated such that it encodes and expresses CTGF-4, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis. 109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) Proc. Natl. Acad. Sci. USA 76:6606).

Suitable adenoviral vectors useful in the present invention are described, for example, in Kozarsky and Wilson, Cuff. Opin. Genet. Devel. 3:499-503 (1993); Rosenfeld et al., Cell 68:143-155 (1992); Engelhardt et al., Human Genet. Ther. 4:759-769 (1993); Yang et al., Nature Genet. 7:362-369 (1994); Wilson et al., Nature 365:691-692 (1993); and U.S. Pat. No. 5,652,224, which are herein incorporated by reference. For example, the adenovirus vector Ad2 is useful and can be grown in human 293 cells. These cells contain the E1 region of adenovirus and constitutively express E1a and E1b, which complement the defective adenoviruses by providing the, products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, for example, the HARP promoter of the present invention, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, N., Curr. Topics in Microbiol. Immunol. 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The CTGF-4 polynucleotide construct is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the CTGF-4 polynucleotide construct. These viral particles are then used to transduce eukaryotic cells, either ex vivo or in vivo. The transduced cells will contain the CTGF-4 polynucleotide construct integrated into its genome, and will express CTGF-4.

Another method of gene therapy involves operably associating heterologous control regions and endogenous polynucleotide sequences (e.g. encoding CTGF-4) via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989). This method involves the activation of a gene which is present in the target cells, but which is not normally expressed in the cells, or is expressed at a lower level than desired.

Polynucleotide constructs are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the CTGF-4 desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. The methods are described in more detail below.

The promoter-targeting sequence construct is taken up by cells. Homologous recombination between the construct and the endogenous sequence takes place, such that an endogenous CTGF-4 sequence is placed under the control of the promoter. The promoter then drives the expression of the endogenous CTGF-4 sequence.

The polynucleotides encoding CTGF-4 may be administered along with other polynucleotides encoding other proteins. Such proteins include, but are not limited to, acidic and basic fibroblast growth factors, VEGF-1, VEGF-2, VEGF-3, VEGF-E, PIGF 1 and 2, epidermal growth factor alpha and beta, platelet-derived endothelial cell growth factor, platelet-derived growth factor alpha and beta, tumor necrosis factor alpha, hepatocyte growth factor, insulin like growth factor, colony stimulating factor, macrophage colony stimulating factor, granulocyte/macrophage colony stimulating factor, and nitric oxide synthase.

Preferably, the polynucleotide encoding CTGF-4 contains a secretory signal sequence that facilitates secretion of the protein. Typically, the signal sequence is positioned in the coding region of the polynucleotide to be expressed towards or at the 5′ end of the coding region. The signal sequence may be homologous or heterologous to the polynucleotide of interest and may be homologous or heterologous to the cells to be transfected. Additionally, the signal sequence may be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers (Kaneda et al., Science 243:375 (1989)).

A preferred method of local administration is by direct injection. Preferably, a recombinant molecule of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA 189:11277-11281, 1992, which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian.

Therapeutic compositions of the present invention can be administered to any animal, preferably to mammals and birds. Preferred mammals include humans, dogs, cats, mice, rats, rabbits sheep, cattle, horses and pigs, with humans being particularly preferred.

Uses of CTGF-4 Polypeptides

CTGF-4 polypeptides can be used in numerous ways. The following description should be considered exemplary and utilizes known techniques.

CTGF-4 polypeptides can be used to assay protein levels in a biological sample using antibody-based techniques. For example, protein expression in tissues can be studied with classical immunohistological methods (Jalkanen, M., et al., J. Cell. Biol. 101:976-985 (1985); Jalkanen, M., et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (99mTc), and fluorescent labels, such as fluorescein, rhodamine, and biotin.

In addition to assaying secreted protein levels in a biological sample, proteins can also be detected in, vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, ¹³¹I, ¹¹²In, ⁹⁹mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹ mTc. The labeled antibody or antibody fragment will then preferentially accumulate at the location of cells which contain the specific protein. In vivo tumor imaging is described by Burchiel and coworkers (“Immunopharmacokinetics of Radiolabeled Antibodies and Their Fragments” (Chapter 13 in Tumor Imaging: The Radiochemical Detection of Cancer, S. W. Burchiel and B. A. Rhodes, eds., Masson Publishing Inc. (1982))).

Thus, the invention provides a diagnostic method of a disorder, which involves (a) assaying the expression of CTGF-4 polypeptide in cells or body fluid of an individual; (b) comparing the level of gene expression with a standard gene expression level, whereby an increase or decrease in the assayed CTGF-4 polypeptide gene expression level compared to the standard expression level is indicative of a disorder.

Moreover, CTGF-4 polypeptides can be used to treat disease. For example, patients can be administered CTGF-4 polypeptides in an effort to replace absent or decreased levels of the CTGF-4 polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, antibodies directed to CTGF-4 polypeptides can also be used to treat disease. For example, administration of an antibody directed to a CTGF-4 polypeptide can bind and reduce overproduction of the polypeptide. Similarly, administration of an antibody can activate the polypeptide, such as by binding to a polypeptide bound to a membrane (receptor).

At the very least, the CTGF-4 polypeptides can be used as molecular weight markers on SDS-PAGE gels or on molecular sieve gel filtration columns using methods well known to those of skill in the art. CTGF-4 polypeptides can also be used to raise antibodies, which in turn are used to measure protein expression from a recombinant cell, as a way of assessing transformation of the host cell. Moreover, CTGF-4 polypeptides can be used to test the following biological activities.

Biological Activities of CTGF-4

CTGF-4 polynucleotides and polypeptides, or agonists or antagonists of CTGF-4, can be used in assays to test for one or more biological activities. If CTGF-4 polynucleotides and polypeptides, or agonists or antagonists of CTGF-4, do exhibit activity in a particular assay, it is likely that CTGF-4 may be involved in the diseases associated with the biological activity. Therefore, CTGF-4 could be used to treat the associated disease.

Isolated CTGF-4 of the present invention can be purified, for instance, as described in Examples 5 and 6, and assayed for biological activity as follows. Since CTGF-4 is a novel growth factor, its ability to stimulate DNA synthesis as measured by [³H]-thymidine incorporation into the DNA of confluent quiescent cell cultures can be measured essentially as described by Brigstock and colleagues (J. Biol. Chem. 272(32):20275-20282 (1997)). Briefly, cultures of Balb/c 3T3 cells (or essentially any human or non-human cell line or primary cell culture) are grown to a state of confluent quiescence in 200 μL of Dulbecco's modified Eagle's medium supplemented with 10% bovine calf serum in 96 well culture plates at 37° C. in an atmosphere of 5% CO₂. Isolated and purified CTGF-4 of the present invention (10-30 μg/mL) is added to the culture medium and the cultures are returned to the incubation conditions described above. After an appropriate incubation time (incubation times can be determined empirically and can range from 10 minutes to 30 minutes to 1 hour to 2 hours to 4 hours to 6 hours to 12 hours to 24 hours to 48 hours), cultures are harvested by scraping, washed several times to remove background signal, and counted for [³H]-thymidine incorporation by liquid scintillation. Potential controls for these assays include 20% calf serum, IGF-1, EGF, bFGF, PDGF-AB, heparin, and combinations thereof.

Immune Activity

CTGF-4 polypeptides or polynucleotides, or agonists or antagonists of CTGF-4, may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B- and T-lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, CTGF-4 polynucleotides or polypeptides, or agonists or antagonists of CTGF-4, can be used as a marker or detector of a particular immune system disease or disorder.

CTGF-4 polynucleotides or polypeptides, or agonists or antagonists of CTGF-4, may be useful in treating or detecting deficiencies or disorders of hematopoietic cells. CTGF-4 polypeptides or polynucleotides, or agonists or antagonists of CTGF-4, could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, CTGF-4 polypeptides or polynucleotides, or agonists or antagonists of CTGF-4, can also be used to modulate hemostatic (the stopping of bleeding) or thrombolytic activity (clot formation). For example, by increasing hemostatic or thrombolytic activity, CTGF-4 polynucleotides or polypeptides, or agonists or antagonists of CTGF-4, could be used to treat blood coagulation disorders (e.g., afibrinogenemia, factor deficiencies), blood platelet disorders (e.g. thrombocytopenia), or wounds resulting from trauma, surgery, or other causes. Alternatively, CTGF-4 polynucleotides or polypeptides, or agonists or antagonists of CTGF-4, that can decrease hemostatic or thrombolytic activity could be used to inhibit or dissolve clotting, important in the treatment of heart attacks (infarction), strokes, or scarring.

CTGF-4 polynucleotides or polypeptides, or agonists or antagonists of CTGF-4, may also be useful in treating or detecting autoimmune disorders. Many autoimmune disorders result from inappropriate recognition of self as foreign material by immune cells. This inappropriate recognition results in an immune response leading to the destruction of the host tissue. Therefore, the administration of CTGF-4 polypeptides or polynucleotides, or agonists or antagonists of CTGF-4, that can inhibit an immune response, particularly the proliferation, differentiation, or chemotaxis of T-cells, may be an effective therapy in preventing autoimmune disorders.

Examples of autoimmune disorders that can be treated or detected by CTGF-4 include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by CTGF-4 polypeptides or polynucleotides, or agonists or antagonists of CTGF-4. Moreover, CTGF-4, or agonists or antagonists of CTGF-4, can be used to treat anaphylaxis, hypersensitivity to an antigenic molecule, or blood group incompatibility.

CTGF-4 polynucleotides or polypeptides, or agonists or antagonists of CTGF-4, may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of CTGF-4 polypeptides or polynucleotides, or agonists or antagonists of CTGF-4, that inhibits an immune response, particularly the proliferation, differentiation, or chemotaxis of T-cells, may be an effective therapy in preventing organ rejection or GVHD.

Similarly, CTGF-4 polypeptides or polynucleotides, or agonists or antagonists of CTGF-4, may also be used to modulate inflammation. For example, CTGF-4 polypeptides or polynucleotides, or agonists or antagonists of CTGF-4, may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1).

Hyperproliferative Disorders

CTGF-4 polypeptides or polynucleotides, or agonists or antagonists of CTGF-4, can be used to treat or detect hyperproliferative disorders, including neoplasms. CTGF-4 polypeptides or polynucleotides may inhibit the proliferation of the disorder through direct or indirect interactions. Alternatively, CTGF-4 polypeptides or polynucleotides, or agonists or antagonists of CTGF-4, may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by CTGF-4 polynucleotides or polypeptides, or agonists or antagonists of CTGF-4, include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated or detected by CTGF-4 polynucleotides or polypeptides, or agonists or antagonists of CTGF-4. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

CTGF-4 can be used to suppress the in vivo growth and metastatic potential of melanoma cells, much in the manner that the highly homologous murine ELM-1 protein can be used to suppress the in vivo growth and metastatic potential of K-1735 mouse melanoma cells (Hashimoto, Y., et al., J. Exp. Med. 187(3):289-296 (1998)).

CTGF-4 can be used to modulate the activities of TGF-beta or other growth factors, cytokines, and chemokines. CTGF-4 has a high degree of sequence to CTGF (see FIGS. 2A, 2B, 2C, 2D, and 2E). Grotendorst (Cytokine Growth Factor Rev. 8(3): 171-179 (1997)) demonstrates that CTGF is a cysteine-rich mitogenic peptide that binds heparin and is secreted by fibroblasts after activation with TGF-beta. In the adult mammal, CTGF functions as a downstream mediator of TGF-beta action on connective tissue cells, where it stimulates cell proliferation and extracellular matrix synthesis. CTGF does not appear to act on epithelial cells or immune cells. Based primarily on sequence conservation, CCN family relationships, and expression patterns, CTGF-4 can also be used to modulate the activities of TGF-beta or other growth factors, cytokines, and chemokines, especially in the immune, urinary, digestive, and reproductive system cells and tissues. Because the biological actions of TGF-beta are complex and affect many different cell types, CTGF and CTGF-4 may serve as specific targets for selective intervention in processes involving connective tissue formation during wound repair or fibrotic disorders. Agents that inhibit CTGF-4 or CTGF production or action are therapeutic approaches for the control of fibrotic disorders in humans.

Cardiovascular Disorders

CTGF-4 polynucleotides or polypeptides, or agonists or antagonists of CTGF-4, encoding CTGF-4 may be used to treat cardiovascular disorders, including peripheral artery disease, such as limb ischemia.

Cardiovascular disorders include cardiovascular abnormalities, such as arterio-arterial fistula, arterioyenous fistula, cerebral arterioyenous malformations, congenital heart defects, pulmonary atresia, and Scimitar Syndrome. Congenital heart defects include aortic coarctation, cor triatriatum, coronary vessel anomalies, crisscross heart, dextrocardia, patent ductus arteriosus, Ebstein's anomaly, Eisenmenger complex, hypoplastic left heart syndrome, levocardia, tetralogy of fallot, transposition of great vessels, double outlet right ventricle, tricuspid atresia, persistent truncus arteriosus, and heart septal defects, such as aortopulmonary septal defect, endocardial cushion defects, Lutembacher's Syndrome, trilogy of Fallot, ventricular heart septal defects.

Cardiovascular disorders also include heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, Scimitar Syndrome, cardiovascular syphilis, and cardiovascular tuberculosis.

Arrhythmias include sinus arrhythmia, atrial fibrillation, atrial flutter, bradycardia, extrasystole, Adams-Stokes Syndrome, bundle-branch block, sinoatrial block, long QT syndrome, parasystole, Lown-Ganong-Levine Syndrome, Mahaim-type pre-excitation syndrome, Wolff-Parkinson-White syndrome, sick sinus syndrome, tachycardias, and ventricular fibrillation. Tachycardias include paroxysmal tachycardia, supraventricular tachycardia, accelerated idioventricular rhythm, atrioventricular nodal reentry tachycardia, ectopic atrial tachycardia, ectopic junctional tachycardia, sinoatrial nodal reentry tachycardia, sinus tachycardia, Torsades de Pointes, and ventricular tachycardia.

Heart valve disease include aortic valve insufficiency, aortic valve stenosis, hear murmurs, aortic valve prolapse, mitral valve prolapse, tricuspid valve prolapse, mitral valve insufficiency, mitral valve stenosis, pulmonary atresia, pulmonary valve insufficiency, pulmonary valve stenosis, tricuspid atresia, tricuspid valve insufficiency, and tricuspid valve stenosis.

Myocardial diseases include alcoholic cardiomyopathy, congestive cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Kearns Syndrome, myocardial reperfusion injury, and myocarditis.

Myocardial ischemias include coronary disease, such as angina pectoris, coronary aneurysm, coronary arteriosclerosis, coronary thrombosis, coronary vasospasm, myocardial infarction and myocardial stunning.

Cardiovascular diseases also include vascular diseases such as aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular disorders, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

Aneurysms include dissecting aneurysms, false aneurysms, infected aneurysms, ruptured aneurysms, aortic aneurysms, cerebral aneurysms, coronary aneurysms, heart aneurysms, and iliac aneurysms.

Arterial occlusive diseases include arteriosclerosis, intermittent claudication, carotid stenosis, fibromuscular dysplasias, mesenteric vascular occlusion, Moyamoya disease, renal artery obstruction, retinal artery occlusion, and thromboanguitis obliterans.

Cerebrovascular disorders include carotid artery diseases, cerebral amyloid angiopathy, cerebral aneurysm, cerebral anoxia, cerebral arteriosclerosis, cerebral arterioyenous malformation, cerebral artery diseases, cerebral embolism and thrombosis, carotid artery thrombosis, sinus thrombosis, Wallenberg's syndrome, cerebral hemorrhage, epidural hematoma, subdural hematoma, subaraxhnoid hemorrhage, cerebral infarction, cerebral ischemia (including transient), subclavian steal syndrome, periventricular leukomalacia, vascular headache, cluster headache, migraine, and vertebrobasilar insufficiency.

Embolisms include air embolisms, amniotic fluid embolisms, cholesterol embolisms, blue toe syndrome, fat embolisms, pulmonary embolisms, and thromoboembolisms. Thrombosis include coronary thrombosis, hepatic vein thrombosis, retinal vein occlusion, carotid artery thrombosis, sinus thrombosis, Wallenberg's syndrome, and thrombophlebitis.

Ischemia includes cerebral ischemia, ischemic colitis, compartment syndromes, anterior compartment syndrome, myocardial ischemia, reperfusion injuries, and peripheral limb ischemia. Vasculitis includes aortitis, arteritis, Behcet's Syndrome, Churg-Strauss Syndrome, mucocutaneous lymph node syndrome, thromboangiitis obliterans, hypersensitivity vasculitis, Schoenlein-Henoch purpura, allergic cutaneous vasculitis, and Wegener's granulomatosis.

CTGF-4 polynucleotides or polypeptides, or agonists or antagonists of CTGF-4, are especially effective for the treatment of critical limb ischemia and coronary disease. As shown in the Examples, administration of CTGF-4 polynucleotides and polypeptides to an experimentally induced ischemia rabbit hindlimb may restore blood pressure ratio, blood flow, angiographic score, and capillary density.

CTGF-4 polypeptides may be administered using any method known in the art, including, but not limited to, direct needle injection at the delivery site, intravenous injection, topical administration, catheter infusion, biolistic injectors, particle accelerators, gelfoam sponge depots, other commercially available depot materials, osmotic pumps, oral or suppositorial solid pharmaceutical formulations, decanting or topical applications during surgery, aerosol delivery. Such methods are known in the art. CTGF-4 polypeptides may be administered as part of a pharmaceutical composition, described in more detail below. Methods of delivering CTGF-4 polynucleotides are described in more detail herein.

Angiogenesis Activity

The naturally occurring balance between endogenous stimulators and inhibitors of angiogenesis is one in which inhibitory influences predominate. Rastinejad et al., Cell 56:345-355 (1989). In those rare instances in which neovascularization occurs under normal physiological conditions, such as wound healing, organ regeneration, embryonic development, and female reproductive processes, angiogenesis is stringently regulated and spatially and temporally delimited. Under conditions of pathological angiogenesis such as that characterizing solid tumor growth, these regulatory controls fail. Unregulated angiogenesis becomes pathologic and sustains progression of many neoplastic and non-neoplastic diseases. A number of serious diseases are dominated by abnormal neovascularization including solid tumor growth and metastases, arthritis, some types of eye disorders, and psoriasis. See, e.g., reviews by Moses et al., Biotech. 9:630-634 (1991); Folkman et al., N. Engl. J. Med., 333:1757-1763 (1995); Auerbach et al., J. Microvasc. Res. 29:401-411 (1985); Folkman, Advances in Cancer Research, eds. Klein and Weinhouse, Academic Press, New York, pp. 175-203 (1985); Patz, Am. J. Opthalmol. 94:715-743 (1982); and Folkman et al., Science 221:719-725 (1983). In a number of pathological conditions, the process of angiogenesis contributes to the disease state. For example, significant data have accumulated which suggest that the growth of solid tumors is dependent on angiogenesis. Folkman and Klagsbrun, Science 235:442-447 (1987).

The present invention provides for treatment of diseases or disorders associated with neovascularization by administration of the CTGF-4 polynucleotides and/or polypeptides of the invention, as well as agonists or antagonists of CTGF-4. Malignant and metastatic conditions which can be treated with the polynucleotides and polypeptides, or agonists or antagonists of the invention include, but are not limited to, malignancies, solid tumors, and cancers described herein and otherwise known in the art (for a review of such disorders, see Fishman et al., Medicine, 2d Ed., J. B. Lippincott Co., Philadelphia (1985)):

Ocular disorders associated with neovascularization which can be treated with the CTGF-4 polynucleotides and polypeptides of the present invention (including CTGF-4 agonists and/or antagonists) include, but are not limited to: neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization. See, e.g., reviews by Waltman et al., Am. J. Ophthal. 85:704-710 (1978) and Gartner et al., Surv. Ophthal. 22:291-312 (1978).

Additionally, disorders which can be treated with the CTGF-4 polynucleotides and polypeptides of the present invention (including CTGF-4 agonists and/or antagonists) include, but are not limited to, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderma, trachoma, and vascular adhesions.

Moreover, disorders and/or states, which can be treated with be treated with the CTGF-4 polynucleotides and polypeptides of the present invention (including CTGF-4 agonists and/or antagonists) include, but are not limited to, solid tumors, blood born tumors such as leukemias, tumor metastasis, Kaposi's sarcoma, benign tumors, for example hemangiomas, acoustic neuromas, neurofibromas, trachomas, and pyogenic granulomas, rheumatoid arthritis, psoriasis, ocular angiogenic diseases, for example, diabetic retinopathy, retinopathy of prematurity, macular degeneration, corneal graft rejection, neovascular glaucoma, retrolental fibroplasia, rubeosis, retinoblastoma, and uvietis, delayed wound healing, endometriosis, vascluogenesis, granulations, hypertrophic scars (keloids), nonunion fractures, scieroderma, trachoma, vascular adhesions, myocardial angiogenesis, coronary collaterals, cerebral collaterals, arterioyenous malformations, ischemic limb angiogenesis, Osler-Webber Syndrome, plaque neovascularization, telangiectasia, hemophiliac joints, angiofibroma fibromuscular dysplasia, wound granulation, Crohn's disease, atherosclerosis, birth control agent by preventing vascularization required for embryo implantation controlling menstruation, diseases that have angiogenesis as a pathologic consequence such as cat scratch disease (Rochele minalia quintosa), ulcers (Helicobacter pylori), Bartonellosis and bacillary angiomatosis.

Diseases at the Cellular Level

Diseases associated with increased cell survival or the inhibition of apoptosis that could be treated or detected by CTGF-4 polynucleotides or polypeptides, as well as antagonists or agonists of CTGF-4, include cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) and viral infections (such as herpes viruses, pox viruses and adenoviruses), inflammation, graft v. host disease, acute graft rejection, and chronic graft rejection. In preferred embodiments, CTGF-4 polynucleotides, polypeptides, and/or antagonists of the invention are used to inhibit growth, progression, and/or metasis of cancers, in particular those listed above.

Additional diseases or conditions associated with increased cell survival that could be treated or detected by CTGF-4 polynucleotides or polypeptides, or agonists or antagonists of CTGF-4, include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Diseases associated with increased apoptosis that could be treated or detected by CTGF-4 polynucleotides or polypeptides, as well as agonists or antagonists of CTGF-4, include AIDS; neurodegenerative disorders (such as Alzheimer's disease, Parkinson's disease, Amyotrophic lateral sclerosis, Retinitis pigmentosa, Cerebellar degeneration and brain tumor or prior associated disease); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (e.g., hepatitis related liver injury, ischemia/reperfusion injury, cholestosis (bile duct injury) and liver cancer); toxin-induced liver disease (such as that caused by alcohol), septic shock, cachexia and anorexia.

Wound Healing and Epithelial Cell Proliferation

In accordance with yet a further aspect of the present invention, there is provided a process for utilizing CTGF-4 polynucleotides or polypeptides, as well as agonists or antagonists of CTGF-4, for therapeutic purposes, for example, to stimulate epithelial cell proliferation and basal keratinocytes for the purpose of wound healing, and to stimulate hair follicle production and healing of dermal wounds. CTGF-4 polynucleotides or polypeptides, as well as agonists or antagonists of CTGF-4, may be clinically useful in stimulating wound healing including surgical wounds, excisional wounds, deep wounds involving damage of the dermis and epidermis, eye tissue wounds, dental tissue wounds, oral cavity wounds, diabetic ulcers, dermal ulcers, cubitus ulcers, arterial ulcers, venous stasis ulcers, burns resulting from heat exposure or chemicals, and other abnormal wound healing conditions such as uremia, malnutrition, vitamin deficiencies and complications associated with systemic treatment with steroids, radiation therapy and antineoplastic drugs and antimetabolites. CTGF-4 polynucleotides or polypeptides, as well as agonists or antagonists of CTGF-4, could be used to promote dermal reestablishment subsequent to dermal loss

CTGF-4 polynucleotides or polypeptides, as well as agonists or antagonists of CTGF-4, could be used to increase the adherence of skin grafts to a wound bed and to stimulate re-epithelialization from the wound bed. The following are types of grafts that CTGF-4 polynucleotides or polypeptides, agonists or antagonists of CTGF-4, could be used to increase adherence to a wound bed: autografts, artificial skin, allografts, autodermic graft, autoepdermic grafts, avacular grafts, Blair-Brown grafts, bone graft, brephoplastic grafts, cutis graft, delayed graft, dermic graft, epidermic graft, fascia graft, full thickness graft, heterologous graft, xenograft, homologous graft, hyperplastic graft, lamellar graft, mesh graft, mucosal graft, Ollier-Thiersch graft, omenpal graft, patch graft, pedicle graft, penetrating graft, split skin graft, thick split graft. CTGF-4 polynucleotides or polypeptides, as well as agonists or antagonists of CTGF-4, can be used to promote skin strength and to improve the appearance of aged skin.

It is believed that CTGF-4 polynucleotides or polypeptides, as well as agonists or antagonists of CTGF-4, will also produce changes in hepatocyte proliferation, and epithelial cell proliferation in the lung, breast, pancreas, stomach, small intestine, and large. intestine. CTGF-4 polynucleotides or polypeptides, as well as agonists or antagonists of CTGF-4, could promote proliferation of epithelial cells such as sebocytes, hair follicles, hepatocytes, type II pneumocytes, mucin-producing goblet cells, and other epithelial cells and their progenitors contained within the skin, lung, liver, and gastrointestinal tract. CTGF-4 polynucleotides or polypeptides, agonists or antagonists of CTGF-4, may promote proliferation of endothelial cells, keratinocytes, and basal keratinocytes.

CTGF-4 polynucleotides or polypeptides, as well as agonists or antagonists of CTGF-4, could also be used to reduce the side effects of gut toxicity that result from radiation, chemotherapy treatments or viral infections. CTGF-4 polynucleotides or polypeptides, as well as agonists or antagonists of CTGF-4, may have a cytoprotective effect on the small intestine mucosa. CTGF-4 polynucleotides or polypeptides, as well as agonists or antagonists of CTGF-4, may also stimulate healing of mucositis (mouth ulcers) that result from chemotherapy and viral infections.

CTGF-4 polynucleotides or polypeptides, as well as agonists or antagonists of CTGF-4, could further be used in full regeneration of skin in full and partial thickness skin defects, including burns, (i.e., repopulation of hair follicles, sweat glands, and sebaceous glands), treatment of other skin defects such as psoriasis. CTGF-4 polynucleotides or polypeptides, as well as agonists or antagonists of CTGF-4, could be used to treat epidermolysis bullosa, a defect in adherence of the epidermis to the underlying dermis which results in frequent, open and painful blisters by accelerating reepithelialization of these lesions. CTGF-4 polynucleotides or polypeptides, as well as agonists or antagonists of CTGF-4, could also be used to treat gastric and doudenal ulcers and help heal by scar formation of the mucosal lining and regeneration of glandular mucosa and duodenal mucosal lining more rapidly. Inflamamatory bowel diseases, such as Crohn's disease and ulcerative colitis, are diseases which result in destruction of the mucosal surface of the small or large intestine, respectively. Thus, CTGF-4 polynucleotides or polypeptides, as well as agonists or antagonists of CTGF-4, could be used to promote the resurfacing of the mucosal surface to aid more rapid healing and to prevent progression of inflammatory bowel disease. Treatment with CTGF-4 polynucleotides or polypeptides, agonists or antagonists of CTGF-4, is expected to have a significant effect on the production of mucus throughout the gastrointestinal tract and could be used to protect the intestinal mucosa from injurious substances that are ingested or following surgery. CTGF-4 polynucleotides or polypeptides, as well as agonists or antagonists of CTGF-4, could be used to treat diseases associate with the under expression of CTGF-4.

Moreover, CTGF-4 polynucleotides or polypeptides, as well as agonists or antagonists of CTGF-4, could be used to prevent and heal damage to the lungs due to various pathological states. A growth factor such as CTGF-4 polynucleotides or polypeptides, as well as agonists or antagonists of CTGF-4, which could stimulate proliferation and differentiation and promote the repair of alveoli and brochiolar epithelium to prevent or treat acute or chronic lung damage. For example, emphysema, which results in the progressive loss of aveoli, and inhalation injuries, i.e., resulting from smoke inhalation and burns, that cause necrosis of the bronchiolar epithelium and alveoli could be effectively treated using CTGF-4 polynucleotides or polypeptides, agonists or antagonists of CTGF-4. Also, CTGF-4 polynucleotides or polypeptides, as well as agonists or antagonists of CTGF-4, could be used to stimulate the proliferation of and differentiation of type II pneumocytes, which may help treat or prevent disease such as hyaline membrane diseases, such as infant respiratory distress syndrome and bronchopulmonary displasia, in premature infants.

CTGF-4 polynucleotides or polypeptides, as well as agonists or antagonists of CTGF-4, could stimulate the proliferation and differentiation of hepatocytes and, thus, could be used to alleviate or treat liver diseases and pathologies such as fulminant liver failure caused by cirrhosis, liver damage caused by viral hepatitis and toxic substances (i.e., acetaminophen, carbon tetraholoride and other hepatotoxins known in the art).

In addition, CTGF-4 polynucleotides or polypeptides, as well as agonists or antagonists of CTGF-4, could be used treat or prevent the onset of diabetes mellitus. In patients with newly diagnosed Types I and II diabetes, where some islet cell function remains, CTGF-4 polynucleotides or polypeptides, as well as agonists or antagonists of CTGF-4, could be used to maintain the islet function so as to alleviate, delay or prevent permanent manifestation of the disease. Also, CTGF-4 polynucleotides or polypeptides, as well as agonists or antagonists of CTGF-4, could be used as an auxiliary in islet cell transplantation to improve or promote islet cell function.

Infectious Disease

CTGF-4 polypeptides or polynucleotides, or agonists or antagonists of CTGF-4, can be used to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, CTGF-4 polypeptides or polynucleotides, or agonists or antagonists of CTGF-4, may also directly inhibit the infectious agent, without necessarily eliciting an immune response.

Viruses are one example of an infectious agent that can cause disease or symptoms that can be treated or detected by CTGF-4 polynucleotides or polypeptides, or agonists or antagonists of CTGF-4. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picomaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. CTGF-4 polypeptides or polynucleotides, or agonists or antagonists of CTGF-4, can be used to treat or detect any of these symptoms or diseases.

Similarly, bacterial or fungal agents that can cause disease or symptoms and that can be treated or detected by CTGF-4 polynucleotides or polypeptides, or agonists or antagonists of CTGF-4, include, but not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. CTGF-4 polypeptides or polynucleotides, or agonists or antagonists of CTGF-4, can be used to treat or detect any of these symptoms or diseases.

Moreover, parasitic agents causing disease or symptoms that can be treated or detected by CTGF-4 polynucleotides or polypeptides, or agonists or antagonists of CTGF-4, include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. CTGF-4 polypeptides or polynucleotides can be used to treat or detect any of these symptoms or diseases.

Preferably, treatment using CTGF-4 polypeptides or polynucleotides, or agonists or antagonists of CTGF-4, could either be by administering an effective amount of CTGF-4 polypeptide, or agonists or antagonists of CTGF-4, to the patient, or by removing cells from the patient, supplying the cells with CTGF-4 polynucleotide, or agonists or antagonists of CTGF-4, and returning the engineered cells to the patient (ex vivo therapy). Moreover, the CTGF-4 polypeptide or polynucleotide, or agonists or antagonists of CTGF-4, can be used as an antigen in a vaccine to raise an immune response against infectious disease.

Regeneration

CTGF-4 polynucleotides or polypeptides, or agonists or antagonists of CTGF-4, can be used to differentiate, proliferate, and attract cells, leading to the regeneration of tissues (see, Science 276:59-87 (1997)). The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vascular (including vascular endothelium), nervous, hematopoietic, and skeletal (bone, cartilage, tendon, and ligament) tissue. Preferably, regeneration occurs without or decreased scarring. Regeneration also may include angiogenesis.

Moreover, CTGF-4 polynucleotides or polypeptides, or agonists or antagonists of CTGF-4, may increase regeneration of tissues difficult to heal. For example, increased tendon/ligament regeneration would quicken recovery time after damage. CTGF-4 polynucleotides or polypeptides, or agonists or antagonists of CTGF-4, of the present invention could also be used prophylactically in an effort to avoid damage. Specific diseases that could be treated include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using CTGF-4 polynucleotides or polypeptides, or agonists or antagonists of CTGF-4, to proliferate and differentiate nerve cells. Diseases that could be treated using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated using the CTGF-4 polynucleotides or polypeptides, or agonists or antagonists of CTGF-4.

Chemotaxis

CTGF-4 polynucleotides or polypeptides may have chemotaxis activity. A chemotactic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

CTGF-4 polynucleotides or polypeptides, or agonists or antagonists of CTGF-4, may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. As a chemotactic molecule, CTGF-4, or agonists or antagonists of CTGF-4, could also attract fibroblasts, which can be used to treat wounds.

It is also contemplated that CTGF-4 polynucleotides or polypeptides, or agonists or antagonists of CTGF-4, may inhibit chemotactic activity. These molecules could also be used to treat disorders. Thus, CTGF-4 polynucleotides or polypeptides, or agonists or antagonists of CTGF-4, could be used as an inhibitor of chemotaxis.

Binding Activity

CTGF-4 polypeptides may be used to screen for molecules that bind to CTGF-4 or for molecules to which CTGF-4 bind. The binding of CTGF-4 and the molecule may activate (agonist), increase, inhibit (antagonist), or decrease activity of the CTGF-4 or the molecule bound. Examples of such molecules include antibodies, oligonucleotides, proteins (e.g., receptors), or small molecules.

Preferably, the molecule is closely related to the natural ligand of CTGF-4, e.g., a fragment of the ligand, or a natural substrate, a ligand, a structural or functional mimetic (See, Coligan, et al., Current Protocols in Immunology 1(2):Chapter 5 (1991)). Similarly, the molecule can be closely related to the natural receptor to which CTGF-4 binds, or at least, a fragment of the receptor capable of being bound by CTGF-4 (e.g., active site). In either case, the molecule can be rationally designed using known techniques.

Preferably, the screening for these molecules involves producing appropriate cells which express CTGF-4, either as a secreted protein or on the cell membrane. Preferred cells include cells from mammals, yeast, Drosophila, or E. coli. Cells expressing CTGF-4 (or cell membrane containing the expressed polypeptide) are then preferably contacted with a test compound potentially containing the molecule to observe binding, stimulation, or inhibition of activity of either CTGF-4 or the molecule.

The assay may simply test binding of a candidate compound to CTGF-4, wherein binding is detected by a label, or in an assay involving competition with a labeled competitor. Further, the assay may test whether the candidate compound results in a signal generated by binding to CTGF-4.

Alternatively, the assay can be carried out using cell-free preparations, polypeptide/molecule affixed to a solid support, chemical libraries, or natural product mixtures. The assay may also simply comprise the steps of mixing a candidate compound with a solution containing CTGF-4, measuring CTGF-4/molecule activity or binding, and comparing the CTGF-4/molecule activity or binding to a standard.

Preferably, an ELISA assay can measure CTGF-4 level or activity in a sample (e.g., biological sample) using a monoclonal or polyclonal antibody. The antibody can measure CTGF-4 level or activity by either binding, directly or indirectly, to CTGF-4 or by competing with CTGF-4 for a substrate.

Additionally, the receptor to which CTGF-4 binds can be identified by numerous methods known to those of skill in the art, for example, ligand panning and FACS sorting (Coligan, et al., Current Protocols in Immun., 1(2), Chapter 5, (1991)). For example, expression cloning is employed wherein polyadenylated RNA is prepared from a cell responsive to the polypeptides, for example, NIH3T3 cells which are known to contain multiple receptors for the FGF family proteins, and SC-3 cells, and a cDNA library created from this RNA is divided into pools and used to transfect COS cells or other cells that are not responsive to the polypeptides. Transfected cells which are grown on glass slides are exposed to the polypeptide of the present invention, after they have been labelled. The polypeptides can be labeled by a variety of means including iodination or inclusion of a recognition site for a site-specific protein kinase.

Following fixation and incubation, the slides are subjected to auto-radiographic analysis. Positive pools are identified and sub-pools are prepared and re-transfected using an iterative sub-pooling and re-screening process, eventually yielding a single clones that encodes the putative receptor.

As an alternative approach for receptor identification, the labeled polypeptides can be photoaffinity linked with cell membrane or extract preparations that express the receptor molecule. Cross-linked material is resolved by PAGE analysis and exposed to X-ray film. The labeled complex containing the receptors of the polypeptides can be excised, resolved into peptide fragments, and subjected to protein microsequencing. The amino acid sequence obtained from microsequencing would be used to design a set of degenerate oligonucleotide probes to screen a cDNA library to identify the genes encoding the putative receptors.

Additionally, this invention provides a method of screening compounds to identify those which modulate the action of the polypeptide of the present invention. An example of such an assay comprises combining a mammalian fibroblast cell, a the polypeptide of the present invention, the compound to be screened and [³H]-thymidine under cell culture conditions where the fibroblast cell would normally proliferate. A control assay may be performed in the absence of the compound to be screened and compared to the amount of fibroblast proliferation in the presence of the compound to determine if the compound stimulates proliferation by determining the uptake of [3H]-thymidine in each case. The amount of fibroblast cell proliferation is measured by liquid scintillation chromatography which measures the incorporation of [³H]-thymidine. Both agonist and antagonist compounds may be identified by this procedure.

In another method, a mammalian cell or membrane preparation expressing a receptor for a polypeptide of the present invention is incubated with a labeled polypeptide of the present invention in the presence of the compound. The ability of the compound to enhance or block this interaction could then be measured. Alternatively, the response of a known second messenger system following interaction of a compound to be screened and the CTGF-4 receptor is measured and the ability of the compound to bind to the receptor and elicit a second messenger response is measured to determine if the compound is a potential agonist or antagonist. Such second messenger systems include but are not limited to, cAMP guanylate cyclase, ion channels or phosphoinositide hydrolysis.

All of these above assays can be used as diagnostic or prognostic markers. The molecules discovered using these assays can be used to treat disease or to bring about a particular result in a patient (e.g., blood vessel growth) by activating or inhibiting the CTGF-4/molecule. Moreover, the assays can discover agents which may inhibit or enhance the production of CTGF-4 from suitably manipulated cells or tissues. Therefore, the invention includes a method of identifying compounds which bind to CTGF-4 comprising the steps of: (a) incubating a candidate binding compound with CTGF-4;, and (b) determining if binding has occurred. Moreover, the invention includes a method of identifying agonists/antagonists comprising the steps of: (a) incubating a candidate compound with CTGF-4, (b) assaying a biological activity, and (b) determining if a biological activity of CTGF-4 has been altered.

Also, one could identify molecules that bind CTGF-4 experimentally by using the beta-pleated sheet regions disclosed in FIG. 3 and Table 1. Accordingly, specific embodiments of the invention are directed to polynucleotides encoding polypeptides which comprise, or alternatively consist of, the amino acid sequence of each beta pleated sheet regions disclosed in FIG. 3 and/or Table 1. Additional embodiments of the invention are directed to polynucleotides encoding CTGF-4 polypeptides which comprise, or alternatively consist of, any combination or all of the beta pleated sheet regions disclosed in FIG. 3 and/or Table 1. Additional preferred embodiments of the invention are directed to polypeptides which comprise, or alternatively consist of, the CTGF-4 amino acid sequence of each of the beta pleated sheet regions disclosed in FIG. 3 and/or Table 1. Additional embodiments of the invention are directed to CTGF-4 polypeptides which comprise, or alternatively consist of, any combination or all of the beta pleated sheet regions disclosed in FIG. 3 and/or Table 1.

Antisense And Ribozyme (Antagonists)

In specific embodiments, antagonists according to the present invention are nucleic acids corresponding to the sequences contained in SEQ ID NO:1, or the complementary strand thereof, and/or to nucleotide sequences contained in the deposited clone 209816. In one embodiment, antisense sequence is generated internally by the organism, in another embodiment, the antisense sequence is separately administered (see, for example, O'Connor, J., Neurochem. 56:560 (1991). Oligodeoxynucleotides as Anitsense Inhibitors of Gene Expression, CRC Press, Boca Raton, Fla. (1988). Antisense technology can be used to control gene expression through antisense DNA or RNA, or through triple-helix formation. Antisense techniques are discussed for example, in Okano, J., Neurochem. 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research 6:3073 (1979); Cooney et al., Science 241:456 (1988); and Dervan et al., Science 251:1300 (1991). The methods are based on binding of a polynucleotide to a complementary DNA or RNA.

For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of the receptor. The antisense RNA oligonucleotide hybridizes to the mRNA in vivo and blocks translation of the mRNA molecule into receptor polypeptide.

In one embodiment, the CTGF-4 antisense nucleic acid of the invention is produced intracellularly by transcription from an exogenous sequence. For example, a vector or a portion thereof, is transcribed, producing an antisense nucleic acid (RNA) of the invention. Such a vector would contain a sequence encoding the CTGF-4 antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others know in the art, used for replication and expression in vertebrate cells. Expression of the sequence encoding CTGF-4, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature 29:304-310 (1981), the promoter contained in the 3′ long terminal repeat of Rous sarcoma virus (Yamamoto et al., Cell 22:787-797 (1980), the herpes thymidine promoter (Wagner et al., Proc. Natl. Acad. Sci. U.S.A. 78:1441-1445 (1981), the regulatory sequences of the metallothionein gene (Brinster, et al., Nature 296:39-42 (1982)), etc.

The antisense nucleic acids of the invention comprise a sequence complementary to at least a portion of an RNA transcript of a CTGF-4 gene. However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded CTGF-4 antisense nucleic acids, a single strand of the duplex DNA may thus be tested, or triplex formation may be assayed. The ability to hybridize will depend on both the degree of complementarity and the length of the antisense nucleic acid Generally, the larger the hybridizing nucleic acid, the more base mismatches with a CTGF-4 RNA it may contain and still form a stable duplex (or triplex as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of CTGF-4 shown in FIGS. 1A, 1B, 1C, and 1D could be used in an antisense approach to inhibit translation of endogenous CTGF-4 mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of CTGF-4 mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

The polynucleotides of the invention can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an a-anomeric oligonucleotide. An a-anomeric oligonucleotide forms specific double-stranded hybrids with complementary RNA in which, contrary to the usual b-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Polynucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

While antisense nucleotides complementary to the CTGF-4 coding region sequence could be used, those complementary to the transcribed untranslated region are most preferred.

Potential antagonists according to the invention also include catalytic RNA, or a ribozyme (See, e.g., PCT International Publication WO 90/11364, published Oct. 4, 1990; Sarver et al, Science 247:1222-1225 (1990). While ribozymes that cleave mRNA at site specific recognition sequences can be used to destroy CTGF-4 mRNAs, the use of hammerhead ribozymes is preferred. Hamunerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). There are numerous potential hammerhead ribozyme cleavage sites within the nucleotide sequence of CTGF-4 (FIGS. 1A, 1B, 1C, and 1D). Preferably, the ribozyme is engineered so that the cleavage recognition site is located near the 5′ end of the CTGF-4 mRNA; i.e., to increase efficiency and minimize the intracellular accumulation of non-functional mRNA transcripts.

As in the antisense approach, the ribozymes of the invention can be composed of modified oligonucleotides (e.g. for improved stability, targeting, etc.) and should be delivered to cells which express CTGF-4 in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter, such as, for example, pol III or pol II promoter, so that transfected cells will produce sufficient quantities of the ribozyme to destroy endogenous CTGF-4 messages and inhibit translation. Since ribozymes unlike antisense molecules, are catalytic, a lower intracellular concentration is required for efficiency.

Antagonist/agonist compounds may be employed to inhibit the cell growth and proliferation effects of the polypeptides of the present invention on neoplastic cells and tissues, i.e. stimulation of angiogenesis of tumors, and, therefore, retard or prevent abnormal cellular growth and proliferation, for example, in tumor formation or growth.

The antagonist/agonist may also be employed to prevent hyper-vascular diseases, and prevent the proliferation of epithelial lens cells after extracapsular cataract surgery. Prevention of the mitogenic activity of the polypeptides of the present invention may also be desirous in cases such as restenosis after balloon angioplasty.

The antagonist/agonist may also be employed to prevent the growth of scar tissue during wound healing.

The antagonist/agonist may also be employed to treat the diseases described herein.

All of these above assays can be used as diagnostic or prognostic markers. The molecules discovered using these assays can be used to treat disease or to bring about a particular result in a patient (e.g., blood vessel growth) by activating or inhibiting the CTGF-4/molecule. Moreover, the assays can discover agents which may inhibit or enhance the production of CTGF-4 from suitably manipulated cells or tissues.

Therefore, the invention includes a method of identifying compounds which bind to CTGF-4 comprising the steps of: (a) incubating a candidate binding compound with CTGF-4; and (b) determining if binding has occurred. Moreover, the invention includes a method of identifying agonists/antagonists comprising the steps of: (a) incubating a candidate compound with CTGF-4, (b) assaying a biological activity, and (b) determining if a biological activity of CTGF-4 has been altered.

Other Activities

CTGF-4 polypeptides or polynucleotides may also increase or decrease the differentiation or proliferation of embryonic stem cells, besides, as discussed above, hematopoietic lineage.

CTGF-4 polypeptides or polynucleotides may also be used to modulate mammalian characteristics, such as body height, weight, hair color, eye color, skin, percentage of adipose tissue, pigmentation, size, and shape (e.g., cosmetic surgery). Similarly, CTGF-4 polypeptides or polynucleotides may be used to modulate mammalian metabolism affecting catabolism, anabolism, processing, utilization, and storage of energy.

CTGF-4 polypeptides or polynucleotides may be used to change a mammal's mental state or physical state by influencing biorhythms, circadic rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

CTGF-4 polypeptides or polynucleotides may also be used as a food additive or preservative, such as to increase or decrease storage capabilities, fat content, lipid, protein, carbohydrate, vitamins, minerals, cofactors or other nutritional components.

Having generally described the invention, the same will be more readily understood by reference to the following examples, which are provided by way of illustration and are not intended as limiting.

### EXAMPLES

### Example 1

### Isolation of the CTGF-4 cDNA Clone From the Deposited Sample

The cDNA for CTGF-4 is inserted into the Eco RI and Xho I restriction sites or other more convenient restriction sites within the multiple cloning site of pBLUESCRIPT (Stratagene, La Jolla, Calif.). pBLUESCRIPT contains an ampicillin resistance gene and may be transformed into E. coli strain DH10B, available from Life Technologies (See, for instance, Gruber, C. E., et al., Focus 15:59 (1993)).

Two approaches can be used to isolate CTGF-4 from the deposited sample. First, a specific polynucleotide of SEQ ID NO: 1 with 30-40 nucleotides is synthesized using an Applied Biosystems DNA synthesizer according to the sequence reported. The oligonucleotide is labeled, for instance, with a-[³²P]-dATP using T4 polynucleotide kinase and purified according to routine methods (e.g., Maniatis, et al., Molecular Cloning: A Laboratory Manual, Cold Spring Harbor Press, Cold Spring, N.Y. (1982)). The plasmid mixture is transformed into a suitable host (such as XL-1 Blue (Stratagene)) using techniques known to those of skill in the art, such as those provided by the vector supplier or in related publications or patents. The transformants are plated on 1.5% agar plates (containing the appropriate selection agent, e.g., ampicillin) to a density of about 150 transformants (colonies) per plate. These plates are screened using Nylon membranes according to routine methods for bacterial colony screening (e.g., Sambrook, et al., Molecular Cloning: A Laboratory Manual, 2nd Edit., (1989), Cold Spring Harbor Laboratory Press, pages 1.93 to 1.104), or other techniques known to those of skill in the art.

Alternatively, two primers of 17-20 nucleotides derived from both ends of the SEQ ID NO:1 (i.e., within the region of SEQ ID NO:1 bounded by the 5′ and 3′ nucleotides of the clone) are synthesized and used to amplify the CTGF-4 cDNA using the deposited cDNA plasmid as a template. The 5′ primer will require the incorporation of the nucleotides 5′-ATG-3′ at immediately upstream of the CTGF-4 coding sequence to incorporate an initiating methionine codon at the 5′ end of the transcribed mRNA molecule (which will, in turn, provide for the incorporation of an N-terminal methionine residue on the CTGF-4 polypeptide chain). The polymerase chain reaction is carried out under routine conditions, for instance, in 25 Al of reaction mixture with 0.5 μg of the above cDNA template. A convenient reaction mixture is 1.5-5 mM MgCl₂, 0.01% (w/v) gelatin, 20 μM each of dATP, dCTP, dGTP, dTTP, 25 pmol of each primer and 0.25 Unit of Taq polymerase. Thirty five cycles of PCR (denaturation at 94° C. for 1 min; annealing at 55° C. for 1 min; elongation at 72° C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Several methods are available for the identification of the 5′ or 3′ non-coding portions of the CTGF-4 gene which may not be present in the deposited clone. These methods include but are not limited to, filter probing, clone enrichment using specific probes, and protocols similar or identical to 5′ and 3′ “RACE” protocols which are well known in the art. For instance, a method similar to 5′ RACE is available for generating the missing 5′ end of a desired full-length transcript (Fromont-Racine, et al., Nucl. Acids Res. 21(7):1683-1684 (1993)).

Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the CTGF-4 gene of interest is used to PCR amplify the 5′ portion of the CTGF-4 full-length gene. This amplified product may then be sequenced and used to generate the full length gene.

This above method starts with total RNA isolated from the desired source, although poly-A+ RNA can be used. The RNA preparation can then be treated with phosphatase if necessary to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the CTGF-4 gene.

### Example 2

### Isolation of CTGF-4 Genomic Clones

A human genomic P1 library (Genomic Systems, Inc.) is screened by PCR using primers selected for the cDNA sequence corresponding to SEQ ID NO: 1., according to the method described in Example 1 (See also, Sambrook, et al., supra).

### Example 3

### Tissue Distribution of CTGF-4 Polypeptides

Tissue distribution of mRNA expression of CTGF-4 is determined using protocols for Northern blot analysis, described by, among others, Sambrook and coworkers (supra). For example, a CTGF-4 probe produced by the method described in Example 1 is labeled with a-[³²P]-dATP using the rediprime™ DNA labeling system (Amersham Life Science), according to manufacturer's instructions. After labeling, the probe is purified using CHROMA SPIN-100™ column (Clontech Laboratories, Inc.), according to manufacturer's protocol number PT1200-1. The purified labeled probe is then used to examine various human tissues for mRNA expression.

Multiple Tissue Northern (MTN) blots containing various human tissues (H) or human immune system tissues (IM) (Clontech) are examined with the labeled probe using Hybrizol hybridization buffer overnight at 42° C. essentially according to the manufacturer's protocol. Following hybridization, the blots are washed three times in 0.1% SDS, 0.2×SSC (once at 42° C. and then twice at 65° C.). Finally, the blots are mounted and exposed to film at −70° C. overnight, and the films developed according to standard procedures. Results of such Northern blot experiments are described above and are shown in FIG. 4.

### Example 4

### Chromosomal Mapping of CTGF-4

An oligonucleotide primer set is designed according to the sequence at the 5′ end of SEQ ID NO:1. This primer preferably spans about 100 nucleotides. This primer set is then used in a polymerase chain reaction under the following set of conditions: 30 seconds, 95° C.; 1 minute, 56° C.; 1 minute, 70° C. This cycle is repeated 32 times followed by one 5 minute cycle at 70° C. Human, mouse, and hamster DNA is used as template in addition to a somatic cell hybrid panel containing individual chromosomes or chromosome fragments (Bios, Inc). The reactions is analyzed on either 8% polyacrylamide gels or 3.5% agarose gels. Chromosome mapping is determined by the presence of an approximately 100 bp PCR fragment in the particular somatic cell hybrid.

### Example 5

### Bacterial Expression of CTGF-4

CTGF-4 polynucleotide encoding a CTGF-4 polypeptide invention is amplified using PCR oligonucleotide primers corresponding to the 5′ and 3′ ends of the DNA sequence, as outlined in Example 1, to synthesize insertion fragments. The primers used to amplify the cDNA insert should preferably contain restriction sites, such as Bam HI and Xba I, at the 5′ end of the primers in order to clone the amplified product into the expression vector. For example, Bam HI and Xba I correspond to the restriction enzyme sites on the bacterial expression vector pQE-9. (Qiagen, Inc., Chatsworth, Calif.). This plasmid vector encodes antibiotic resistance (AmpR), a bacterial origin of replication (ori), an IPTG-regulatable promoter/operator (P/O), a ribosome binding site (RBS), a 6-histidine tag (6-His), and restriction enzyme cloning sites.

Specifically, to clone the full-length CTGF-4 molecule contained in cDNA clones HWHGU74 and HWHGU74S15 in ATCC Deposit No. 209816 (an overlap PCR product of which is nearly the predicted mature CTGF-4 based on homology with murine ELM-1 (FIGS. 2A, 2B, 2C, 2D, and 2E)) in a bacterial vector, the 5′ primer has the sequence 5′-CGC \ddot{GGA TCC} GCG ATG GAC TTT ACC CCA GCT CC-3′ (SEQ ID NO:13) containing the underlined Bam HI restriction site followed a methionine codon and 17 nucleotides of the amino terminal coding sequence of the nearly mature CTGF-4 sequence in SEQ ID NO:1. One of ordinary skill in the art would appreciate, of course, that the point in the protein coding sequence where the 5′ primer begins may be varied to amplify a DNA segment encoding any desired portion of the complete CTGF-4 protein shorter or longer than the nearly mature domain of the protein. The 3′ primer has the sequence 5′-CTA GTC TAG A CT AGG TTG GCA ATT TCT GAG AAG TCA GGG-3′ (SEQ ID NO: 14) containing the underlined Xba I restriction site followed by 25 nucleotides complementary to the 3′ end of the coding sequence of the CTGF-4 DNA sequence of SEQ ID NO: 1.

The pQE-9 vector is digested with Bam HI and Xba I and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, which expresses the lacI repressor and also confers kanamycin resistance (Kan^{R}). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 μg/ml) and Kan (25 μg/ml). The O/N culture is used to inoculate a large culture at a ratio of 1:100 to 1:250. The cells are grown to an optical density 600 (O.D.₆₀₀) of between 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 M Guanidine HCl by stirring for 3-4 hours at 4° C. The cell debris is removed by centrifugation, and the supernatant containing the polypeptide is applied to a nickel-nitrilo-tri-acetic acid (“Ni-NTA”) affinity resin column (available from QIAGEN, Inc., supra). Proteins with a 6×His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified CTGF-4 protein is then renatured by dialyzing it against phosphate-buffered saline (PBS) or 50 mM Na-acetate, pH 6 buffer plus 200 mM NaCl. Alternatively, the CTGF-4 protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM immidazole. Immidazole is removed by a final dialyzing step against PBS or 50 mM sodium acetate pH 6 buffer plus 200 mM NaCl. The purified CTGF-4 protein is stored at 4° C. or frozen at −80° C.

In addition to the above expression vector, the present invention further includes an expression vector comprising phage operator and promoter elements operatively linked to a CTGF-4 polynucleotide, called pHE4a (ATCC Accession Number 209645, deposited Feb. 25, 1998). This vector contains: 1) a neomycinphosphotransferase gene as a selection marker, 2) an E. coli origin of replication, 3) a T5 phage promoter sequence, 4) two lac operator sequences, 5) a Shine-Delgarno sequence, and 6) the lactose operon repressor gene (lacIq). The origin of replication (oriC) is derived from pUC19 (LTI, Gaithersburg, Md.). The promoter sequence and operator sequences are made synthetically.

DNA can be inserted into the pHEa by restricting the vector with Nde I and Xba I, Bam HI, Xho I, or Asp 718, running the restricted product on a gel, and isolating the larger fragment (the stuffer fragment should be about 310 base pairs). The DNA insert is generated according to the PCR protocol described in Example 1, using PCR primers having a methionine codon and an Nde I restriction site (5′ primer) and an Xba I, Bam HI, Xho I or Asp 718 restriction site (3′ primer). The PCR insert is gel purified and restricted with compatible enzymes. The insert and vector are ligated according to standard protocols.

The engineered vector could easily be substituted in the above protocol to express protein in a bacterial system.

### Example 6

### Purification of CTGF-4 Polypeptide from an Inclusion Body

The following alternative method can be used to purify CTGF-4 polypeptide expressed in E coli when it is present in the form of inclusion bodies. Unless otherwise specified, all of the following steps are conducted at 4-10° C.

Upon completion of the production phase of the E. coli fermentation, the cell culture is cooled to 4-10° C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5 M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min, the pellet is discarded and the polypeptide containing supernatant is incubated at 4° C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded polypeptide solution, a previously prepared tangential filtration unit equipped with 0.16 (m membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the CTGF-4 polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant CTGF-4 polypeptide should exhibit greater than 95% purity after the above refolding and purification steps. No major contaminant bands should be observed from Commassie blue stained 16% SDS-PAGE gel when 5 μg of purified protein is loaded. The purified CTGF-4 protein can also be tested for endotoxin/LPS contamination, and typically the LPS content is less than 0.1 ng/ml according to LAL assays.

### Example 7

### Cloning and Expression of CTGF-4 in a Baculovirus Expression System

In this example, the plasmid shuttle vector pA2GP is used to insert CTGF-4 polynucleotide into a baculovirus to express CTGF-4. This expression vector contains the strong polyhedrin promoter of the Autographa californica nuclear polyhedrosis virus (AcMNPV) followed by convenient restriction sites such as Bam HI, Xba I and Asp 718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned CTGF-4 polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required (see, for instance, Luckow, et al., Virology 170:31-39 (1989)).

Specifically, the CTGF-4 cDNA sequence contained in the deposited clones (ATCC Deposit No. 209816) is amplified using the PCR protocol described in Example 1. An initiating methionine and baculovirus signal peptide are provided by the pA2GP vector (Summers, et al., “A Manual of Methods for Baculovirus Vectors and Insect Cell Culture Procedures,” Texas Agricultural Experimental Station Bulletin No. 1555 (1987)).

More specifically, the cDNA sequence encoding the full-length CTGF-4 protein in the deposited clone is amplified using PCR oligonucleotide primers corresponding to the 5′ and 3′ sequences of the gene. The 5′ primer has the sequence 5′-CGC GGA TCC GCG CGA CTT TAC CCC AGC TCC-3′ (SEQ ID NO:15) containing the Bam HI restriction enzyme site and four non-coding restriction site flanking residues to preserve the reading frame, followed by 17 nucleotides of the sequence of the complete CTGF-4 protein shown in FIGS. 1A, 1B, 1C, and 1D, beginning with the aspartic acid codon (GAC). The 3′ primer has the sequence 5′-CTA GGG TAC CCT AGG TTG GCA ATT TCT GAG AAG TCA GGG-3′ (SEQ ID NO:16) containing the Asp 718 restriction site followed by a number of nucleotides complementary to the 3′ noncoding sequence in FIGS. 1A, 1B, 1C, and 1D.

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five micrograms of a plasmid containing the polynucleotide is co-transfected with 1.0 micrograms of a commercially available linearized baculovirus DNA (“BaculoGold™ baculovirus DNA”, Pharmingen, San Diego, Calif.), using the lipofection method described by Felgner and colleagues (Proc. Natl. Acad. Sci. USA 84:7413-7417 (1987)). One microgram of BaculoGold™ virus DNA and 5 micrograms of the plasmid are mixed in a sterile well of a microtiter plate containing 50 microliters of serum-free Grace's medium (Life Technologies Inc., Gaithersburg, Md.). Afterwards, 10 microliters Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith (supra.). An agarose gel with “Blue Gal” (Life Technologies Inc., Gaithersburg) is used to allow easy identification and isolation of gal-expressing clones, which produce blue-stained plaques (a detailed description of a “plaque assay” of this type can also be found in the user's guide for insect cell culture and baculovirology distributed by Life Technologies Inc., Gaithersburg, page 9-10). After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced CTGF-4 protein.

### Example 8

### Expression of CTGF-4 in Mammalian Cells

CTGF-4 polypeptide can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, CTGF-4 polypeptide can be expressed in stable cell lines containing the CTGF-4 polynucleotide integrated into a chromosome. The co-transfection with a selectable marker such as dhfr, gpt, neomycin, hygromycin allows the identification and isolation of the transfected cells.

The transfected CTGF-4 gene can also be amplified to express large amounts of the encoded protein. The DHFR (dihydrofolate reductase) marker is useful in developing cell lines that carry several hundred or even several thousand copies of the gene of interest (see, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991)). Another useful selection marker is the enzyme glutamine synthase (GS; Murphy, et al., Biochem J. 227:277-279 (1991); Bebbington, et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-dhfr (ATCC Accession No. 37146), the expression vectors pC4 (ATCC Accession No. 209646) and pC6 (ATCC Accession No.209647) contain the strong promoter (LTR) of the Rous Sarcoma Virus (Cullen, et al., Mol. Cell. Biol., 438-447 (1985)) plus a fragment of the CMV-enhancer (Boshart et al., Cell 41:521-530 (1985)). Multiple cloning sites, e.g., with the restriction enzyme cleavage sites Bam HI, Xba I and Asp 718, facilitate the cloning of CTGF-4. The vectors also contain the 3′ intron, the polyadenylation and termination signal of the rat preproinsulin gene, and the mouse DHFR gene under control of the SV40 early promoter.

Specifically, the plasmid pC6, for example, is digested with appropriate restriction enzymes and then dephosphorylated using calf intestinal phosphates by procedures known in the art. The vector is then isolated from a 1% agarose gel.

CTGF-4 polynucleotide is amplified according to the protocol outlined in Example 1. If the naturally occurring signal sequence is used to produce the secreted protein, the vector does not need a second signal peptide. Alternatively, if the naturally occurring signal sequence is not used, the vector can be modified to include a heterologous signal sequence (see, e.g., WO 96/34891).

The amplified fragment is isolated from a 1% agarose gel using a commercially available kit (“Geneclean”, BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transfection. Five μg of the expression plasmid pC6 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of CTGF-4 is analyzed, for instance, by SDS-PAGE and Western blot or by reversed phase HPLC analysis.

### Example 9

### Protein Fusions of CTGF-4

CTGF-4 polypeptides are preferably fused to other proteins. These fusion proteins can be used for a variety of applications. For example, fusion of CTGF-4 polypeptides to His-tag, HA-tag, protein A, IgG domains, and maltose binding protein facilitates purification (see Example 5; see also EP A 394,827; Traunecker, et al., Nature 331:84-86 (1988)). Similarly, fusion to IgG-1, IgG-3, and albumin increases the halflife time in vivo. Nuclear localization signals fused to CTGF-4 polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule, or the protocol described in Example 5.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (ATCC Accession No. 209646) is used, the human Fc portion can be ligated into the Bam HI cloning site. Note that the 3′ Bam HI site should be destroyed. Next, the vector containing the human Fc portion is re-restricted with Bam HI, linearizing the vector, and CTGF-4 polynucleotide, isolated by the PCR protocol described in Example 1, is ligated into this Bam HI site. Note that the CTGF-4 PCR product produced as described in Example 1 requires the addition of a methionine codon as described in Example 5 and that the vector must be modified to include a heterologous signal sequence (see, e.g., WO 96/34891). In addition, note that the polynucleotide is cloned without a stop codon, otherwise a fusion protein will not be produced.

Human IgG Fc region:
(SEQ ID NO:17)
GGGATCCGGAGCCCAAATCTTCTGACAAAACTCACACATGCCCACCGTGCCCAGCACCTGAATTCGAGGG
TGCACCGTCAGTCTTCCTCTTCCCCCCAAAACCCAAGGACACCCTCATGATCTCCCGGACTCCTGAGGTCACATGCGTGG
TGGTGGACGTAAGCCACGAAGACCCTGAGGTCAAGTTCAACTGGTACGTGGACGGCGTGGAGGTGCATAATGCCAAGACA
AAGCCGCGGGAGGAGCAGTACAACAGCACGTACCGTGTGGTCAGCGTCCTCACCGTCCTGCACCAGGACTGGCTGAATGG
CAAGGAGTACAAGTGCAAGGTCTCCAACAAAGCCCTCCCAACCCCCATCGAGAAAACCATCTCCAAAGCCAAAGGGCAGC
CCCGAGAACCACAGGTGTACACCCTGCCCCCATCCCGGGATGAGCTGACCAAGAACCAGGTCAGCCTGACCTGCCTGGTC
AAAGGCTTCTATCCAAGCGACATCGCCGTGGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTCCGGGTAAATGAGTG
CGACGGCCGCGACTCTAGAGGAT

### Example 10

### Production of an Antibody

The antibodies of the present invention can be prepared by a variety of methods (see, Current Protocols, Chapter 2). For example, cells expressing CTGF-4 are administered to an animal to induce the production of sera containing polyclonal antibodies. In a preferred method, a preparation of CTGF-4 protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology (Kohler, et al., Nature 256:495 (1975); Kohler, et al., Eur. J. Immunol. 6:511 (1976); Kohler, et al., Eur. J. Immunol. 6:292 (1976); Hammerling, et al., in: Monoclonal Antibodies and T-Cell Hybridomas, Elsevier, N.Y., pp. 563-681 (1981)). In general, such procedures involve immunizing an animal (preferably a mouse) with CTGF-4 polypeptide or, more preferably, with a secreted CTGF-4 polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/i of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands and coworkers (Gastroenterology 80:225-232 (1981)). The hybridoma cells obtained through such a selection are then assayed to identify clones which secrete antibodies capable of binding the CTGF-4 polypeptide.

Alternatively, additional antibodies capable of binding to CTGF-4 polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the CTGF-4 protein-specific antibody can be blocked by CTGF-4. Such antibodies comprise anti-idiotypic antibodies to the CTGF-4 protein-specific antibody and can be used to immunize an animal to induce formation of further CTGF-4 protein-specific antibodies.

It will be appreciated that Fab and F(ab′)₂ and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments). Alternatively, secreted CTGF-4 protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art (see, for review, Morrison, Science 229:1202 (1985); Oi, et al., BioTechniques 4:214 (1986); Cabilly, et al., U.S. Pat. No. 4,816,567; Taniguchi, et al., EP 171496; Morrison, et al., EP 173494; Neuberger, et al., WO 8601533; Robinson, et al., WO 8702671; Boulianne, et al., Nature 312:643 (1984); Neuberger, et al., Nature 314:268 (1985)).

### Example 11

### Production of CTGF-4 Protein for High-Throughput Screening Assays

The following protocol produces a supernatant containing CTGF-4 polypeptide to be tested. This supernatant can then be used in the Screening Assays described in Examples 13-20.

First, dilute Poly-D-Lysine (644 587 Boehringer-Mannheim) stock solution (1 mg/ml in PBS) 1:20 in PBS (w/o calcium or magnesium 17-516F Biowhittaker) for a working solution of 50 mg/ml. Add 200 ml of this solution to each well (24 well plates) and incubate at RT for 20 minutes. Be sure to distribute the solution over each well (note: a 12-channel pipetter may be used with tips on every other channel). Aspirate off the Poly-D-Lysine solution and rinse with 1 ml PBS (Phosphate Buffered Saline). The PBS should remain in the well until just prior to plating the cells and plates may be poly-lysine coated in advance for up to two weeks.

Plate 293T cells (do not carry cells past P+20) at 2×10⁵ cells/well in 0.5 ml DMEM (Dulbecco's Modified Eagle Medium; with 4.5 G/L glucose and L-glutamine (12-604F Biowhittaker))/10% heat inactivated FBS (14-503F Biowhittaker)/ 1×Penstrep (17-602E Biowhittaker). Let the cells grow overnight.

The next day, mix together in a sterile solution basin: 300 ml Lipofectamine (18324-012 Gibco/BRL) and 5 ml Optimem 1 (31985070 Gibco/BRL)/96-well plate. With a small volume multi-channel pipetter, aliquot approximately 2 mg of an expression vector containing a polynucleotide insert, produced by the methods described in Examples 8 or 9, into an appropriately labeled 96-well round bottom plate. With a multi-channel pipetter, add 50 ml of the Lipofectamine/Optimem I mixture to each well. Pipette up and down gently to mix. Incubate at RT 15-45 minutes. After about 20 minutes, use a multi-channel pipetter to add 150 ul Optimem I to each well. As a control, one plate of vector DNA lacking an insert should be transfected with each set of transfections.

Preferably, the transfection should be performed by tag-teaming the following tasks. By tag-teaming, hands on time is cut in half, and the cells do not spend too much time on PBS. First, person A aspirates off the media from four 24-well plates of cells, and then person B rinses each well with 0.5-1.0 ml PBS. Person A then aspirates off PBS rinse, and person B, using a 12-channel pipetter with tips on every other channel, adds the 200 ml of DNA/Lipofectamine/Optimem I complex to the odd wells first, then to the even wells, to each row on the 24-well plates. Incubate at 37° C. for 6 hours.

While cells are incubating, prepare appropriate media, either 1% BSA in DMEM with 1×penstrep, or HGS CHO-5 media (116.6 mg/L of CaCl2 (anhyd); 0.00130 mg/L CuSO₄-5H₂O; 0.050 mg/L of Fe(NO₃)₃-9H₂O; 0.417 mg/L of FeSO₄-7H₂O; 311.80 mg/L of Kcl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄-H₂O; 71.02 mg/L of Na₂HPO4; 0.4320 mg/L of ZnSO₄-7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L-Arginine-HCL; 7.50 mg/ml of L-Asparagine-H₂O; 6.65 mg/ml of L-Aspartic Acid; 29.56 mg/ml of L-Cystine-2HCL-H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalainine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; and 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; 0.680 mg/L of Vitamin B₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; 10 mg/L of Methyl-B-Cyclodextrin complexed with Retinal Acetate. Adjust osmolarity to 327 mOsm) with 2 mm glutamine and 1×penstrep. (BSA (81-068-3 Bayer) 100 gm dissolved in IL DMEM for a 10% BSA stock solution). Filter the media and collect 50 ml for endotoxin assay in 15 ml polystyrene conical.

The transfection reaction is terminated, preferably by tag-teaming, at the end of the incubation period. Person A aspirates off the transfection media, while person B adds 1.5 ml appropriate media to each well. Incubate at 37° C. for 45 or 72 hours depending on the media used: 1% BSA for 45 hours or CHO-5 for 72 hours.

On day four, using a 300 ml multichannel pipetter, aliquot 600 ml in one 1 ml deep well plate and the remaining supernatant into a 2 ml deep well. The supernatants from each well can then be used in the assays described in Examples 13-20.

It is specifically understood that when activity is obtained in any of the assays described below using a supernatant, the activity originates from either the CTGF-4 polypeptide directly (e.g., as a secreted protein) or by CTGF-4 inducing expression of other proteins, which are then secreted into the supernatant. Thus, the invention further provides a method of identifying the protein in the supernatant characterized by an activity in a particular assay.

### Example 12

### Construction of GAS Reporter Construct

One signal transduction pathway involved in the differentiation and proliferation of cells is called the Jaks-STATs pathway. Activated proteins in the Jaks-STATs pathway bind to gamma activation site “GAS” elements or interferon-sensitive responsive element (“ISRE”), located in the promoter of many genes. The binding of a protein to these elements alter the expression of the associated gene.

GAS and ISRE elements are recognized by a class of transcription factors called Signal Transducers and Activators of Transcription, or “STATs”. There are six members of the STATs family. Stat1 and Stat3 are present in many cell types, as is Stat2 (as response to IFN-alpha is widespread). Stat4 is more restricted and is not in many cell types though it has been found in T helper class I, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jak1, Jak2, and Jak3. These kinases display significant sequence similarity and are generally catalytically inactive in resting cells.

The Jaks are activated by a wide range of receptors summarized in the Table below (adapted from review by Schidler and Darnell, Ann. Rev. Biochem. 64:621-51 (1995)). A cytokine receptor family, capable of activating Jaks, is divided into two groups: (a) Class 1 includes receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; and (b) Class 2 includes IFN-a, IFN-g, and IL-10. The Class 1 receptors share a conserved cysteine motif (a set of four conserved cysteines and one tryptophan) and a WSXWS motif (a membrane proxial region encoding Trp-Ser-Xxx-Trp-Ser (SEQ ID NO: 18)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate proteins involved in the proliferation and differentiation of cells. For example, growth factors and cytokines are known to activate the Jaks-STATs pathway (see Table below). Thus, by using GAS elements linked to reporter molecules, activators of the Jaks-STATs pathway can be identified.

| | | JAKs | | | | |
|---|---|---|---|---|---|---|
| Ligand | tyk2 | Jak1 | Jak2 | Jak3 | STATS | GAS(elements) or ISRE |

IFN family

| | | | | | | |
|---|---|---|---|---|---|---|
| IFN-a/B | + | + | − | − | 1,2,3 | ISRE |
| IFN-g | | + | + | − | 1 | GAS (IRF1 > Lys6 > IFP) |
| Il-10 | + | ? | ? | − | 1,3 | |
| gp130 family | | | | | | |
| IL-6 (Pleiotrohic) | + | + | + | ? | 1,3 | GAS (IRF1 > Lys6 > IFP) |
| Il-11 (Pleiotrohic) | ? | + | ? | ? | 1,3 | |
| OnM (Pleiotrohic) | ? | + | + | ? | 1,3 | |
| LIF (Pleiotrohic) | ? | + | + | ? | 1,3 | |
| CNTF (Pleiotrohic) | −/+ | + | + | ? | 1,3 | |
| G-CSF (Pleiotrohic) | ? | + | ? | ? | 1,3 | |
| IL-12 (Pleiotrohic) | + | − | + | + | 1,3 | |
| g-C family | | | | | | |
| IL-2 (lymphocytes) | − | + | − | + | 1,3,5 | GAS |
| IL-4 (lymph/myeloid) | − | + | − | + | 6 | GAS (IRF1 = IFP >> Ly6)(IgH) |
| IL-7 (lymphocytes) | − | + | − | + | 5 | GAS |
| IL-9 (lymphocytes) | − | + | − | + | 5 | GAS |
| IL-13 (lymphocyte) | − | + | ? | ? | 6 | GAS |
| IL-15 | ? | + | ? | + | 5 | GAS |
| gp140 family | | | | | | |
| IL-3 (myeloid) | − | − | + | − | 5 | GAS (IRF1 > IFP >> Ly6) |
| IL-5 (myeloid) | − | − | + | − | 5 | GAS |
| GM-CSF (myeloid) | − | − | + | − | 5 | GAS |
| Growth hormone family | | | | | | |
| GH | ? | − | + | − | 5 | |
| PRL | ? | +/− | + | − | 1,3,5 | |
| EPO | ? | − | + | − | 5 | GAS (B − CAS > IRF1 = IFP >> Ly6) |
| Receptor Tyrosine Kinases | | | | | | |
| EGF | ? | + | + | − | 1,3 | GAS (IRF1) |
| PDGF | ? | + | + | − | 1,3 | |
| CSF-1 | ? | + | + | − | 1,3 | GAS (not IRF1) |

To construct a synthetic GAS containing promoter element, which is used in the Biological Assays described in Examples 13-14, a PCR based strategy is employed to generate a GAS-SV40 promoter sequence. The 5′ primer contains four tandem copies of the GAS binding site found in the IRF1 promoter and previously demonstrated to bind STATs upon induction with a range of cytokines (Rothman, et al., Immunity 1:457-468 (1994)), although other GAS or ISRE elements can be used instead. The 5′ primer also contains 18 bp of sequence complementary to the SV40 early promoter sequence and is flanked with an Xho I site. The sequence of the 5′ primer is: 5′-GCG CCT CGA GAT TTC CCC GAA ATC TAG ATT TCC CCG AAA TGA TTT CCC CGA AAT GAT TTC CCC GAA ATA TCT GCC ATC TCA ATT AG-3′ (SEQ ID NO: 19). The downstream primer is complementary to the SV40 promoter and is flanked with a Hin dIII site. The sequence of the 3′ primer is: 5′-GCG GCA AGC TTT TTG CAA AGC CTA GGC-3′ (SEQ ID NO:20).

PCR amplification is performed using the SV40 promoter template present in the β-gal: promoter plasmid obtained from Clontech. The resulting PCR fragment is digested with Xho I and Hin dIII and subcloned into BLSK2-(Stratagene). Sequencing with forward and reverse primers confirms that the insert contains the following sequence: 5′-CTC GAG ATT TCC CCG AAA TCT AGA TTT CCC CGA AAT GAT TTC CCC GAA ATG ATT TCC CCG AAA TAT CTG CCA TCT CAA TTA GTC AGC AAC CAT AGT CCC GCC CCT AAC TCC GCC CAT CCC GCC CCT AAC TCC GCC CAG TTC CGC CCA TTC TCC GCC CCA TGG CTG ACT AAT TTT TTT TAT TTA TGC AGA GGC CGA GGC CGC CTC GGC CTC TGA GCT ATT CCA GAA GTA GTG AGG AGG CTT TTT TGG AGG CCT AGG CTT TTG CAA AAA GCT T-3′ (SEQ ID NO:21).

With this GAS promoter element linked to the SV40 promoter, a GAS:SEAP2 reporter construct is next engineered. Here, the reporter molecule is a secreted alkaline phosphatase, or “SEAP”. Clearly, however, any reporter molecule can be instead of SEAP, in this or in any of the other Examples. Well-known reporter molecules that can be used instead of SEAP include chloramphenicol acetyltransferase (CAT), luciferase, alkaline phosphatase, β-galactosidase, green fluorescent protein (GFP), or any protein detectable by an antibody.

The above sequence confirmed synthetic GAS-SV40 promoter element is subcloned into the pSEAP-Promoter vector obtained from Clontech using Hin dIII and Xho I, effectively replacing the SV40 promoter with the amplified GAS:SV40 promoter element, to create the GAS-SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using Sal I and Not I, and inserted into a backbone vector containing the neomycin resistance gene, such as pGFP-1 (Clontech), using these restriction sites in the multiple cloning site, to create the GAS-SEAP/Neo vector. Once this vector is transfected into mammalian cells, this vector can then be used as a reporter molecule for GAS binding as described in Examples 13-14.

Other constructs can be made using the above description and replacing GAS with a different promoter sequence. For example, construction of reporter molecules containing NF-κB and EGR promoter sequences are described in Examples 15 and 16. However, many other promoters can be substituted using the protocols described in these Examples. For instance, SRE, IL-2, NFAT, or Osteocalcin promoters can be substituted, alone or in combination (e.g., GAS/NF-κB/EGR, GAS/NF-κB, Il-2/NFAT, or NF-κB/GAS). Similarly, other cell lines can be used to test reporter construct activity, such as HeLa (epithelial), HUVEC (endothelial), Reh (B-cell), Saos-2 (osteoblast), HUVAC (aortic) or cardiomyocyte.

### Example 13

### High-Throughput Screening Assay for T-Cell Activity

The following protocol is used to assess T-cell activity of CTGF-4 by determining whether CTGF-4 supernatant proliferates and/or differentiates T-cells. T-cell activity is assessed using the GAS/SEAP/Neo construct produced in Example 12. Thus, factors that increase SEAP activity indicate the ability to activate the Jaks-STATs signal transduction pathway. The T-cell used in this assay is Jurkat T-cells (ATCC Accession No. TIB-152), although Molt-3 cells (ATCC Accession No. CRL-1552) and Molt-4 cells (ATCC Accession No. CRL-1582) cells can also be used.

Jurkat T-cells are lymphoblastic CD4+ Th1 helper cells. In order to generate stable cell lines, approximately 2 million Jurkat cells are transfected with the GAS-SEAP/neo vector using DMRIE-C (Life Technologies; transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 μl of cells. Thus, it is either scaled up, or performed in multiple to generate sufficient cells for multiple 96 well plates. Jurkat cells are maintained in RPMI+10% serum with 1% Pen-Strep. Combine 2.5 mls of OPTI-MEM (Life Technologies) with 10 μg of plasmid DNA in a T25 flask. Add 2.5 ml OPTI-MEM containing 50 μl of DMRIE-C and incubate at room temperature for 15-45 mins.

During the incubation period, count cell concentration, spin down the required number of cells (107 per transfection), and resuspend in OPTI-MEM to a final concentration of 107 cells/ml. Then add 1 ml of 1×10⁷ cells in OPTI-MEM to T25 flask and incubate at 37° C. for 6 hrs. After the incubation, add 10 ml of RPMI+15% serum.

The Jurkat:GAS-SEAP stable reporter lines are maintained in RPMI+10% serum, 1 mg/ml Genticin, and 1% Pen-Strep. These cells are treated with supernatants containing CTGF-4 polypeptides or CTGF-4 induced polypeptides as produced by the protocol described in Example 11.

On the day of treatment with the supernatant, the cells should be washed and resuspended in fresh RPMI+10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 μl of cells into each well (therefore adding 100,000 cells per well).

After all the plates have been seeded, 50 μl of the supernatants are transferred directly from the 96 well plate containing the supernatants into each well using a 12 channel pipette. In addition, a dose of exogenous interferon gamma (0.1, 1.0, 10 ng) is added to wells H9, H10, and H 1I to serve as additional positive controls for the assay.

The 96 well dishes containing Jurkat cells treated with supernatants are placed in an incubator for 48 hrs (note: this time is variable between 48-72 hrs). 35/1 samples from each well are then transferred to an opaque 96 well plate using a 12 channel pipette. The opaque plates should be covered (using sellophene covers) and stored at −20° C. until SEAP assays are performed according to Example 17. The plates containing the remaining treated cells are placed at 4° C. and serve as a source of material for repeating the assay on a specific well if desired.

As a positive control, 100 Unit/ml interferon gamma can be used which is known to activate Jurkat T cells. Over 30 fold induction is typically observed in the positive control wells.

### Example 14

### High-Throughput Screening Assay Identifying Myeloid Activity

The following protocol is used to assess myeloid activity of CTGF-4 by determining whether CTGF-4 proliferates and/or differentiates myeloid cells. Myeloid cell activity is assessed using the GAS/SEAP/Neo construct produced in Example 12. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 12, a DEAE-Dextran method (Kharbanda, et. al., Cell Growth & Differentiation 5:259-265 (1994)) is used. First, harvest 2×10⁷ U937 cells and wash with PBS. The U937 cells are usually grown in RPMI 1640 medium containing 10% heat-inactivated fetal bovine serum (FBS) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 ug GAS-SEAP2 plasmid DNA, 140 mM NaCl, 5 mM KCl, 375 μM Na₂HPO₄7H₂O, 1 mM MgCl₂, and 675 μM CaCl₂. Incubate at 37° C. for 45 min.

Wash the cells with RPMI 1640 medium containing 10% FBS and then resuspend in 10 ml complete medium and incubate at 37° C. for 36 hr.

The GAS-SEAP/U937 stable cells are obtained by growing the cells in 400 μg/ml G418. The G418-free medium is used for routine growth but every one to two months, the cells should be re-grown in 400 tig/ml G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 μl cells per well in the 96-well plate (or 1×10⁵ cells/well).

Add 50 μl of the supernatant prepared by the protocol described in Example 11. Incubate at 37° C. for 48 to 72 hr. As a positive control, 100 Unit/ml interferon gamma can be used which is known to activate U937 cells. Over 30 fold induction is typically observed in the positive control wells. SEAP assay the supernatant according to the protocol described in Example 17.

### Example 15

### High-Throughput Screening Assay Identifying Neuronal Activity

When cells undergo differentiation and proliferation, a group of genes are activated through many different signal transduction pathways. One of these genes, EGR1 (early growth response gene 1), is induced in various tissues and cell types upon activation. The promoter of EGR1 is responsible for such induction. Using the EGR1 promoter linked to reporter molecules, activation of cells can be assessed by CTGF-4.

Particularly, the following protocol is used to assess neuronal activity in PC12 cell lines. PC12 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PC12 cells with a construct containing an EGR promoter linked to SEAP reporter, activation of PC12 cells by CTGF-4 can be assessed.

The EGR/SEAP reporter construct can be assembled by the following protocol. The EGR-1 promoter sequence (−633 to +1; Sakamoto, K., et al., Oncogene 6:867-871 (1991)) can be PCR amplified from human genomic DNA using the following primers. The 5′ primer has the sequence: 5′-GCG CTC GAG GGA TGA CAG CGA TAG AAC CCC GG-3′ (SEQ ID NO:22) and the 3′ primer has the sequence: 5′-GCG AAG CTT CGC GAC TCC CCG GAT CCG CCT C-3′ (SEQ ID NO:23).

Using the GAS:SEAP/Neo vector produced in Example 12, EGR1 amplified product can then be inserted into this vector. Linearize the GAS:SEAP/Neo vector using restriction enzymes Xho I and Hin dIII, removing the GAS/SV40 stuffer. Restrict the EGR1 amplified product with these same enzymes. Ligate the vector and the EGR1 promoter.

To prepare 96 well-plates for cell culture, two ml of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PC12 cells are routinely grown in RPMI-1640 medium (Bio Whittaker) containing 10% horse serum (JRH BIOSCIENCES, Cat. # 12449-78P), 5% heat-inactivated fetal bovine serum (FBS) supplemented with 100 units/ml penicillin and 100 tg/ml streptomycin on a precoated 10 cm tissue culture dish. One to four split is done every three to four days. Cells are removed from the plates by scraping and resuspended with pipetting up and down for more than 15 times.

Transfect the EGR/SEAP/Neo construct into PC12 using the Lipofectamine protocol described in Example 11. EGR-SEAP/PC 12 stable cells are obtained by growing the cells in 300 μg/ml G418. The G418-free medium is used for routine growth but every one to two months, the cells should be re-grown in 300 μg/ml G418 for couple of passages.

To assay for neuronal activity, a 10 cm plate with cells around 70 to 80% confluent is screened by removing the old medium. Wash the cells once with PBS (Phosphate buffered saline). Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 μl of the cell suspension to each well of 96-well plate (equivalent to 1×105 cells/well). Add 50 μl supernatant produced by Example 11, 37° C. for 48 to 72 hr. As a positive control, a growth factor known to activate PC12 cells through EGR can be used, such as 50 ng/ul of Neuronal Growth Factor (NGF). Over fifty-fold induction of SEAP is typically seen in the positive control wells. SEAP assay the supernatant according to Example 17.

### Example 16

### High-Throughput Screening Assay for T-Cell Activity

NF-kB (Nuclear Factor kB) is a transcription factor activated by a wide variety of agents including the inflammatory cytokines IL-1 and TNF, CD30 and CD40, lymphotoxin-alpha and lymphotoxin-beta, by exposure to LPS or thrombin, and by expression of certain viral gene products. As a transcription factor, NF-kB regulates the expression of genes involved in immune cell activation, control of apoptosis (NF-kB appears to shield cells from apoptosis), B- and T-cell development, anti-viral and antimicrobial responses, and multiple stress responses.

In non-stimulated conditions, NF-kB is retained in the cytoplasm with I-kB (Inhibitor kB). However, upon stimulation, 1-kB is phosphorylated and degraded, causing NF-KB to shuttle to the nucleus, thereby activating transcription of target genes. Target genes activated by NF-KB include IL-2, IL-6, GM-CSF, ICAM-1 and class 1 MHC.

Due to its central role and ability to respond to a range of stimuli, reporter constructs utilizing the NF-kB promoter element are used to screen the supernatants produced in Example 11. Activators or inhibitors of NF-KB would be useful in treating diseases. For example, inhibitors of NF-kB could be used to treat those diseases related to the acute or chronic activation of NF-kB, such as rheumatoid arthritis.

To construct a vector containing the NF-kB promoter element, a PCR based strategy is employed. The 5′ primer contains four tandem copies of the NF-KB binding site (5′-GGG GAC TTT CCC-3′; SEQ ID NO:24), 18 bp of sequence complementary to the 5′ end of the SV40 early promoter sequence, and is flanked with an Xho I site and has the following sequence: 5′-GCG GCC TCG AGG GGA CTT TCC CGG GGA CTT TCC GGG GAC TTT CCG GGA CTT TCC ATC CTG CCA TCT CAA TTA G-3′ (SEQ ID NO:25). The 3′ primer is complementary to the 3′ end of the SV40 promoter, is flanked with a Hin dIII site and has the following sequence: 5′-GCG GCA AGC TTT TTG CAA AGC CTA GGC-3′ (SEQ ID NO:26).

PCR amplification is performed using the SV40 promoter template present in the pbeta-gal:promoter plasmid obtained from Clontech. The resulting PCR fragment is digested with Xho I and Hin dIII and subcloned into BLSK2-(Stratagene). Sequencing with the T7 and T3 primers confirms the insert contains the following sequence: 5′-CTC GAG GGG ACT TTC CCG GGG ACT TTC CGG GGA CTT TCC GGG ACT TTC CAT CTG CCA TCT CAA TTA GTC AGC AAC CAT AGT CCC GCC CCT AAC TCC GCC CAT CCC GCC CCT AAC TCC GCC CAG TTC CGC CCA TTC TCC GCC CCA TGG CTG ACT AAT TTT TTT TAT TTA TGC AGA GGC CGA GGC CGC CTC GGC CTC TGA GCT ATT CCA GAA GTA GTG AGG AGG CTT TTT TGG AGG CCT AGG CTT TTG CAA AAA GCT T-3′ (SEQ ID NO:27).

Next, replace the SV40 minimal promoter element present in the pSEAP2-promoter plasmid (Clontech) with this NF-kB/SV40 fragment using Xho I and Hin dIII. However, this vector does not contain a neomycin resistance gene, and therefore, is not preferred for mammalian expression systems.

In order to generate stable mammalian cell lines, the NF-kB/SV40/SEAP cassette is removed from the above NF-kB/SEAP vector using restriction enzymes Sal I and Not I, and inserted into a vector containing neomycin resistance. Particularly, the NF-kB/SV40/SEAP cassette was inserted into pGFP-1 (Clontech), replacing the GFP gene, after restricting pGFP-1 with Sal I and Not I.

Once NF-kB/SV40/SEAP/Neo vector is created, stable Jurkat T-cells are created and maintained according to the protocol described in Example 13. Similarly, the method for assaying supernatants with these stable Jurkat T-cells is also described in Example 13. As a positive control, exogenous TNF alpha (0.1, 1, 10 ng) is added to wells H9, H10, and H11, with a 5-10 fold activation typically observed.

### Example 17

### Assay for SEAP Activity

As a reporter molecule for the assays described in Examples 13-16, SEAP activity is assayed using the Tropix Phospho-light Kit (Cat. BP-400) according to the following general procedure. The Tropix Phospho-light Kit supplies the Dilution, Assay, and Reaction Buffers used below.

Prime a dispenser with the 2.5×Dilution Buffer and dispense 15 μl of 2.5×dilution buffer into Optiplates containing 35 μl of a supernatant. Seal the plates with a plastic sealer and incubate at 65° C. for 30 min. Separate the Optiplates to avoid uneven heating.

Cool the samples to room temperature for 15 minutes. Empty the dispenser and prime with the Assay Buffer. Add 50 μl Assay Buffer and incubate at room temperature 5 min. Empty the dispenser and prime with the Reaction Buffer (see the table below). Add 50 μl Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 18

### High-Throughput Screening Assay Identifying Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-3, used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 ul of HBSS (Hank's Balanced Salt Solution) leaving 100 μl of buffer after the final wash.

A stock solution of 1 mg/ml fluo-3 is made in 10% pluronic acid DMSO. To load the cells with fluo-3, 50 μl of 12 ug/ml fluo-3 is added to each well. The plate is incubated at 37° C. in a CO₂ incubator for 60 min. The plate is washed four times in the Biotek washer with HBSS leaving 100 μl of buffer.

For non-adherent cells, the cells are spun down from culture media. Cells are re-suspended to 2-5×10⁶ cells/ml with HBSS in a 50-ml conical tube. 4 μl of 1 mg/ml fluo-3 solution in 10% pluronic acid DMSO is added to each ml of cell suspension. The tube is then placed in a 37° C. water bath for 30-60 min. The cells are washed twice with HBSS, resuspended to 1×10⁶ cells/ml, and dispensed into a microplate, 100 μl/well. The plate is centrifuged at 1000 rpm for 5 min. The plate is then washed once in Denley CellWash with 200 ul, followed by an aspiration step to 100 μl final volume.

For a non-cell based assay, each well contains a fluorescent molecule, such as fluo-3. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 μl. Increased emission at 530 nm indicates an extracellular signaling event caused by the a molecule, either CTGF-4 or a molecule induced by CTGF-4, which has resulted in an increase in the intracellular Ca²⁺ concentration.

### Example 19

### High-Throughput Screening Assay Identifying Tyrosine Kinase Activity

The Protein Tyrosine Kinases (PTK) represent a diverse group of transmembrane and cytoplasmic kinases. Within the Receptor Protein Tyrosine Kinase RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small proteins, but also membrane-bound and extracellular matrix proteins.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, lck, lyn, fyn) and non-receptor linked and cytosolic protein tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, identifying whether CTGF-4 or a molecule induced by CTGF-4 is capable of activating tyrosine kinase signal transduction pathways is of interest. Therefore, the following protocol is designed to identify such molecules capable of activating the tyrosine kinase signal transduction pathways.

Seed target cells (e.g., primary keratinocytes) at a density of approximately 25,000 cells per well in a 96 well Loprodyne Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/ml), gelatin (2%) or polylysine (50 mg/ml), all of which can be purchased from Sigma Chemicals (St. Louis, Mo.) or 10% Matrigel purchased from Becton Dickinson (Bedford, Mass.), or calf serum, rinsed with PBS and stored at 4° C. Cell growth on these plates is assayed by seeding 5,000 cells/well in growth medium and indirect quantitation of cell number through use of alamarBlue as described by the manufacturer Alamar Biosciences, Inc. (Sacramento, Calif.) after 48 hr. Falcon plate covers #3071 from Becton Dickinson (Bedford, Mass.) are used to cover the Loprodyne Silent Screen Plates. Falcon Microtest III cell culture plates can also be used in some proliferation experiments.

To prepare extracts, A431 cells are seeded onto the nylon membranes of Loprodyne plates (20,000/200 ml/well) and cultured overnight in complete medium. Cells are quiesced by incubation in serum-free basal medium for 24 hr. After 5-20 minutes treatment with EGF (60 ng/ml) or 50 ul of the supernatant produced in Example 11, the medium was removed and 100 ml of extraction buffer ((20 mM HEPES pH 7.5, 0.15 M NaCl, 1% Triton X-100, 0.1% SDS, 2 mM Na₃VO₄, 2 mM Na₄P₂O₇ and a cocktail of protease inhibitors (#1836170) obtained from Boeheringer Mannheim (Indianapolis, Ind.) is added to each well and the plate is shaken on a rotating shaker for 5 minutes at 4° C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4° C. at 16,000×g.

Test the filtered extracts for levels of tyrosine kinase activity. Although many methods of detecting tyrosine kinase activity are known, one method is described here. Generally, the tyrosine kinase activity of a supernatant is evaluated by determining its ability to phosphorylate a tyrosine residue on a specific substrate (a biotinylated peptide). Biotinylated peptides that can be used for this purpose include PSK1 (corresponding to amino acids 6-20 of the cell division kinase cdc2-p34) and PSK2 (corresponding to amino acids 1-17 of gastrin). Both peptides are substrates for a range of tyrosine kinases and are available from Boehringer Mannheim.

The tyrosine kinase reaction is set up by adding the following components in order. First, add 10 μl of 5 μM Biotinylated Peptide, then 10 μl ATP/Mg²⁺ (5 mM ATP/50 mM MgCl₂), then 10 μl of 5×Assay Buffer (40 mM imidazole hydrochloride, pH 7.3, 40 mM beta-glycerophosphate, 1 mM EGTA, 100 mM MgCl₂, 5 mM MnCl₂, 0.5 mg/ml BSA), then 5 μl of Sodium Vanadate(1 mM), and then 5 μl of water. Mix the components gently and preincubate the reaction mix at 30° C. for 2 min. Initial the reaction by adding 10 μl of the control enzyme or the filtered supernatant. The tyrosine kinase assay reaction is then terminated by adding 10 μl of 120 mm EDTA and place the reactions on ice.

Tyrosine kinase activity is determined by transferring 50 μl aliquot of reaction mixture to a microtiter plate (MTP) module and incubating at 37° C. for 20 min. This allows the streptavadin coated 96 well plate to associate with the biotinylated peptide. Wash the MTP module with 300 μl/well of PBS four times. Next add 75 μl of anti-phospolyrosine antibody conjugated to horse radish peroxidase(anti-P-Tyr-POD(0.5 μ/ml)) to each well and incubate at 37° C. for one hour. Wash the well as above.

Next add 100 μl of peroxidase substrate solution (Boehringer Mannheim) and incubate at room temperature for at least 5 min (up to 30 min). Measure the absorbance of the sample at 405 nm by using ELISA reader. The level of bound peroxidase activity is quantitated using an ELISA reader and reflects the level of tyrosine kinase activity.

### Example 20

### High-Throughput Screening Assay Identifying Phosphorylation Activity

As a potential alternative or complement to the assay of protein tyrosine kinase activity described in Example 19, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), IRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.1 ml of protein G (1 μg/ml) for 2 hr at room temp (RT). The plates are then rinsed with PBS and blocked with 3% BSA/PBS for 1 hr at RT. The protein G plates are then treated with 2 commercial monoclonal antibodies (100 ng/well) against Erk-1 and Erk-2 (1 hr at RT; available from Santa Cruz Biotechnology). To detect other molecules, this step can easily be modified by substituting a monoclonal antibody detecting any of the above described molecules. After 3-5 rinses with PBS, the plates are stored at 4° C. until use.

A431 cells are seeded at 20,000/well in a 96-well Loprodyne filterplate and cultured overnight in growth medium. The cells are then starved for 48 hr in basal medium (DMEM) and then treated with EGF (6 ng/well) or 50 Ill of the supernatants obtained in Example 11 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (1 μg/ml) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation by CTGF-4 or a molecule induced by CTGF-4.

### Example 21

### Method of Determining Alterations in the CTGF-4 Gene

RNA isolated from entire families or individual patients presenting with a phenotype of interest (such as a disease) is be isolated. cDNA is then generated from these RNA samples using protocols known in the art (see, Sambrook, et al., supra) The cDNA is then used as a template for PCR, employing primers surrounding regions of interest in SEQ ID NO:1. Suggested PCR conditions consist of 35 cycles at 95° C. for 30 seconds; 60-120 seconds at 52-58° C.; and 60-120 seconds at 70° C., using buffer solutions described (Sidransky, D., et al., Science 252:706 (1991)).

PCR products are then sequenced using primers labeled at their 5′ end with T4 polynucleotide kinase, employing SequiTherm Polymerase (Epicentre Technologies). The intron-exon borders of selected exons of CTGF-4 is also determined and genomic PCR products analyzed to confirm the results. PCR products harboring suspected mutations in CTGF-4 is then cloned and sequenced to validate the results of the direct sequencing.

PCR products of CTGF-4 are cloned into T-tailed vectors as described (Holton, T. A. and Graham, M. W., Nucl. Acids Res. 19:1156 (1991)) and sequenced with T7 polymerase (United States Biochemical). Affected individuals are identified by mutations in CTGF-4 not present in unaffected individuals.

Genomic rearrangements are also observed as a method of determining alterations in the CTGF-4 gene. Genomic clones isolated according to Example 2 are nick-translated with digoxigenindeoxy-uridine 5′-triphosphate (Boehringer Manheim), and FISH performed as described (Johnson, C., et al., Methods Cell Biol. 35:73-99 (1991)). Hybridization with the labeled probe is carried out using a vast excess of human cot-1 DNA for specific hybridization to the CTGF-4 genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters (Johnson, C., et al., Genet. Anal. Tech. Appl. 8:75 (1991)). Image collection, analysis and chromosomal fractional length measurements are performed using the ISee Graphical Program System (Inovision Corporation, Durham, N.C.). Chromosome alterations of the genomic region of CTGF-4 (hybridized by the probe) are identified as insertions, deletions, and translocations. These CTGF-4 alterations are used as a diagnostic marker for an associated disease.

### Example 22

### Method of Detecting Abnormal Levels of CTGF-4 in a Biological Sample

CTGF-4 polypeptides can be detected in a biological sample, and if an increased or decreased level of CTGF-4 is detected, this polypeptide is a marker for a particular phenotype. Methods of detection are numerous, and thus, it is understood that one skilled in the art can modify the following assay according to specific needs.

For example, antibody-sandwich ELISAs are used to detect CTGF-4 in a sample, preferably a biological sample. Wells of a microtiter plate are coated with specific antibodies to CTGF-4, at a final concentration of 0.2 to 10 μg/ml. The antibodies are either monoclonal or polyclonal and are produced by the method described in Example 10. The wells are blocked so that non-specific binding of CTGF-4 to the well is reduced.

The coated wells are then incubated for greater than 2 hours at RT with a sample containing CTGF-4. Preferably, serial dilutions of the sample should be used to validate results. The plates are then washed three times with deionized or distilled water to remove unbound CTGF-4.

Next, 50 μl of specific antibody-alkaline phosphatase conjugate, at a concentration of 25-400 ng, is added and incubated for 2 hours at room temperature. The plates are again washed three times with deionized or distilled water to remove unbounded conjugate.

Add 75 μl of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot CTGF-4 polypeptide concentration on the X-axis (log scale) and fluorescence or absorbance of the Y-axis (linear scale). Interpolate the concentration of the CTGF-4 in the sample using the standard curve.

### Example 23

### Formulating a Polypeptide

The CTGF-4 composition will be formulated and dosed in a fashion consistent with good medical practice, taking into account the clinical condition of the individual patient (especially the side effects of treatment with the CTGF-4 polypeptide alone), the site of delivery, the method of administration, the scheduling of administration, and other factors known to practitioners. The “effective amount” for purposes herein is thus determined by such considerations.

As a general proposition, the total pharmaceutically effective amount of CTGF-4 administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, CTGF-4 is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing CTGF-4 are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

CTGF-4 is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U., et al., Biopolymers 22:547-556 (1983)), poly (2-hydroxyethyl methacrylate; Langer, R., et al., J. Biomed. Mater. Res. 15:167-277 (1981); Langer, R. Chem. Tech. 12:98-105 (1982)), ethylene vinyl acetate (Langer, R., et al., supra) or poly-D-(−)-3-hydroxybutyric acid (EP 133,988). Sustained-release compositions also include liposomally entrapped CTGF-4 polypeptides. Liposomes containing the CTGF-4 are prepared by methods known per se (DE 3,218,121; Epstein, et al., Proc. Natl. Acad. Sci. USA 82:3688-3692 (1985); Hwang, et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324). Ordinarily, the liposomes are of the small (about 200-800 Angstroms) unilamellar type in which the lipid content is greater than about 30 mol percent cholesterol, the selected proportion being adjusted for the optimal secreted polypeptide therapy.

For parenteral administration, in one embodiment, CTGF-4 is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting CTGF-4 uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

CTGF-4 is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

CTGF-4 used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

CTGF-4 polypeptides ordinarily will be stored in unit or multi-dose containers, for example, sealed ampoules or vials, as an aqueous solution or as a lyophilized formulation for reconstitution. As an example of a lyophilized formulation, 10 ml vials are filled with 5 ml of sterile-filtered 1% (w/v) aqueous CTGF-4 polypeptide solution, and the resulting mixture is lyophilized. The infusion solution is prepared by reconstituting the lyophilized CTGF-4 polypeptide using bacteriostatic Water-For-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, CTGF-4 may be employed in conjunction with other therapeutic compounds.

### Example 24

### Method of Treating Decreased Levels of CTGF-4

The present invention relates to a method for treating an individual in need of a decreased level of CTGF-4 activity in the body comprising, administering to such an individual a composition comprising a therapeutically effective amount of CTGF-4 antagonist. Preferred antagonists for use in the present invention are CTGF-4-specific antibodies.

Moreover, it will be appreciated that conditions caused by a decrease in the standard or normal expression level of CTGF-4 in an individual can be treated by administering CTGF-4, preferably in the secreted or mature form. Thus, the invention also provides a method of treatment of an individual in need of an increased level of CTGF-4 polypeptide comprising administering to such an individual a pharmaceutical composition comprising an amount of CTGF-4 to increase the activity level of CTGF-4 in such an individual.

For example, a patient with decreased levels of CTGF-4 polypeptide receives a daily dose 0.1-100 μg/kg of the polypeptide for six consecutive days. Preferably, the polypeptide is in the secreted form. The exact details of the dosing scheme, based on administration and formulation, are provided in Example 23.

### Example 25

### Method of Treating Increased Levels of CTGF-4

The present invention also relates to a method for treating an individual in need of an increased level of CTGF-4 activity in the body comprising administering to such an individual a composition comprising a therapeutically effective amount of CTGF-4 or an agonist thereof.

Antisense technology is used to inhibit production of CTGF-4. This technology is one example of a method of decreasing levels of CTGF-4 polypeptide, preferably a secreted form, due to a variety of etiologies, such as cancer.

For example, a patient diagnosed with abnormally increased levels of CTGF-4 is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided in Example 23.

### Example 26

### Method of Treatment Using Gene Therapy

One method of gene therapy transplants fibroblasts, which are capable of expressing CTGF-4 polypeptides, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37° C. for approximately one week. At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with Eco RI and Hin dIII and subsequently treated with calf intestinal phosphatase. The linear vector is fractionated on agarose gel and purified, using glass beads.

The cDNA encoding CTGF-4 can be amplified using PCR primers which correspond to the 5′ and 3′ end sequences respectively as set forth in Example 1. Preferably, the 5′ primer contains an Eco RI site and a codon which corresponds to an initiating methionine and the 3′ primer includes a Hin dIII site. Equal quantities of the Moloney murine sarcoma virus linear backbone and the amplified Eco RI and Hin dIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector contains properly inserted CTGF-4.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the CTGF-4 gene is then added to the media and the packaging cells transduced with the vector. The packaging cells now produce infectious viral particles containing the CTGF-4 gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether CTGF-4 protein is produced.

The engineered fibroblasts are then transplanted onto the host, either alone or after having been grown to confluence on cytodex 3 microcarrier beads.

### Example 27

### Method of Treatment Using Gene Therapy—Ex Vivo

One method of gene therapy transplants fibroblasts, which are capable of expressing CTGF-4 polypeptides, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37 degree C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding CTGF-4 can be amplified using PCR primers which correspond to the 5′ and 3′ end sequences respectively as set forth in Example 1. Preferably, the 5′ primer contains an Eco RI site and the 3′ primer includes a Hin dIII site. Equal quantities of the Moloney murine sarcoma virus linear backbone and the amplified Eco RI and Hin dIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector contains properly inserted CTGF-4.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the CTGF-4 gene is then added to the media and the packaging cells transduced with the vector. The packaging cells now produce infectious viral particles containing the CTGF-4 gene(the packaging cells are now referred to as producer cells).

Fresh media is added to the transduced producer cells, and subsequently, the media is harvested from a 10. cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether CTGF-4 protein is produced.

The engineered fibroblasts are then transplanted onto the host, either alone or after having been grown to confluence on cytodex 3 microcarrier beads.

### Example 28

### Gene Therapy Using Endogenous CTGF-4 Gene

Another method of gene therapy according to the present invention involves operably associating the endogenous CTGF-4 sequence with a promoter via homologous recombination as described, for example, in U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989). This method involves the activation of a gene which is present in the target cells, but which is not expressed in the cells, or is expressed at a lower level than desired.

Polynucleotide constructs are made which contain a promoter and targeting sequences, which are homologous to the 5′ non-coding sequence of endogenous CTGF-4, flanking the promoter. The targeting sequence will be sufficiently near the 5′ end of CTGF-4 so the promoter will be operably linked to the endogenous sequence upon homologous recombination. The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter.

The amplified promoter and the amplified targeting sequences are digested with the appropriate restriction enzymes and subsequently treated with calf intestinal phosphatase. The digested promoter and digested targeting sequences are added together in the presence of T4 DNA ligase. The resulting mixture is maintained under conditions appropriate for ligation of the two fragments. The construct is size fractionated on an agarose gel then purified by phenol extraction and ethanol precipitation.

In this Example, the polynucleotide constructs are administered as naked polynucleotides via electroporation. However, the polynucleotide constructs may also be administered with transfection-facilitating agents, such as liposomes, viral sequences, viral particles, precipitating agents, etc. Such methods of delivery are known in the art.

Once the cells are transfected, homologous recombination will take place which results in the promoter being operably linked to the endogenous CTGF-4 sequence. This results in the expression of CTGF-4 in the cell. Expression may be detected by immunological staining, or any other method known in the art.

Fibroblasts are obtained from a subject by skin biopsy. The resulting tissue is placed in DMEM+10% fetal calf serum. Exponentially growing or early stationary phase fibroblasts are trypsinized and rinsed from the plastic surface with nutrient medium. An aliquot of the cell suspension is removed for counting, and the remaining cells are subjected to centrifugation. The supernatant is aspirated and the pellet is resuspended in 5 ml of electroporation buffer (20 mM HEPES pH 7.3, 137 mM NaCl, 5 mM KCl, 0.7 mM Na₂ HPO₄, 6 mM dextrose). The cells are recentrifuged, the supernatant aspirated, and the cells resuspended in electroporation buffer containing 1 mg/ml acetylated bovine serum albumin. The final cell suspension contains approximately 3×10⁶ cells/ml. Electroporation should be performed immediately following resuspension.

Plasmid DNA is prepared according to standard techniques. For example, to construct a plasmid for targeting to the CTGF-4 locus, plasmid pUC18 (MBI Fermentas, Amherst, N.Y.) is digested with Hin dIII. The CMV promoter is amplified by PCR with an XbaI site on the 5′ end and a BamHI site on the 3′end. Two CTGF-4 non-coding sequences are amplified via PCR: one CTGF-4 non-coding sequence (CTGF-4 fragment 1) is amplified with a HindIII site at the 5′ end and an Xba site at the 3′end; the other CTGF-4 non-coding sequence (CTGF-4 fragment 2) is amplified with a BamHI site at the 5′end and a HindIII site at the 3′end. The CMV promoter and CTGF-4 fragments are digested with the appropriate enzymes (CMV promoter—XbaI and BamHI; CTGF-4 fragment 1—XbaI; CTGF-4 fragment 2—BamHI) and ligated together. The resulting ligation product is digested with HindIII, and ligated with the HindIII-digested pUC18 plasmid.

Plasmid DNA is added to a sterile cuvette with a 0.4 cm electrode gap (Bio-Rad). The final DNA concentration is generally at least 120 μg/ml. 0.5 ml of the cell suspension (containing approximately 1.5.×10⁶ cells) is then added to the cuvette, and the cell suspension and DNA solutions are gently mixed. Electroporation is performed with a Gene-Pulser apparatus (Bio-Rad). Capacitance and voltage are set at 960 μF and 250-300 V, respectively. As voltage increases, cell survival decreases, but the percentage of surviving cells that stably incorporate the introduced DNA into their genome increases dramatically. Given these parameters, a pulse time of approximately 14-20 mSec should be observed.

Electroporated cells are maintained at room temperature for approximately 5 min, and the contents of the cuvette are then gently removed with a sterile transfer pipette. The cells are added directly to 10 ml of prewarmed nutrient media (DMEM with 15% calf serum) in a 10 cm dish and incubated at 37° C. The following day, the media is aspirated and replaced with 10 ml of fresh media and incubated for a further 16-24 hours.

The engineered fibroblasts are then injected into the host, either alone or after having been grown to confluence on cytodex 3 microcarrier beads. The fibroblasts now produce the protein product. The fibroblasts can then be introduced into a patient as described above.

### Example 29

### Method of Treatment Using Gene Therapy—In Vivo

Another aspect of the present invention is using in vivo gene therapy methods to treat disorders, diseases and conditions. The gene therapy method relates to the introduction of naked nucleic acid (DNA, RNA, and antisense DNA or RNA) CTGF-4 sequences into an animal to increase or decrease the expression of the CTGF-4 polypeptide. The CTGF-4 polynucleotide may be operatively linked to a promoter or any other genetic elements necessary for the expression of the CTGF-4 polypeptide by the target tissue. Such gene therapy and delivery techniques and methods are known in the art, see, for example, WO90/11092, WO98/11779; U.S. Pat. Nos. 5,693,622, 5,705,151, 5,580,859; Tabata H. et al. (1997) Cardiovasc. Res. 35(3):470-479, Chao J et al. (1997) Pharmacol. Res. 35(6):517-522, Wolff J. A. (1997) Neuromuscul. Disord. 7(5):314-318, Schwartz B. et al. (1996) Gene Ther. 3(5):405-411, Tsurumi Y. et al. (1996) Circulation 94(12):3281-3290 (incorporated herein by reference).

The CTGF-4 polynucleotide constructs may be delivered by any method that delivers injectable materials to the cells of an animal, such as, injection into the interstitial space of tissues (heart, muscle, skin, lung, liver, intestine and the like). The CTGF-4 polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the CTGF-4 polynucleotides may also be delivered in liposome formulations (such as those taught in Felgner P. L. et al. (1995) Ann. NY Acad. Sci. 772:126-139 and Abdallah B. et al. (1995) Biol. Cell 85(1):1-7) which can be prepared by methods well known to those skilled in the art.

The CTGF-4 polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The CTGF-4 polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked CTGF-4 polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked CTGF-4 polynucleotide constructs can be delivered to arteries during angioplasty by the catheter used in the procedure.

The dose response effects of injected CTGF-4 polynucleotide in muscle in vivo is determined as follows. Suitable CTGF-4 template DNA for production of mRNA coding for CTGF-4 polypeptide is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The CTGF-4 template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for CTGF-4 protein expression. A time course for CTGF-4 protein expression may be done in a similar fashion except that quadriceps from different mice are harvested at different times. Persistence of CTGF-4 DNA in muscle following injection may be determined by Southern blot analysis after preparing total cellular DNA and HIT supernatants from injected and control mice. The results of the above experimentation in mice can be use to extrapolate proper dosages and other treatment parameters in humans and other animals using CTGF-4 naked DNA.

### Example 30

### CTGF-4 Transgenic Animals

The CTGF-4 polypeptides can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred.

Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art. The contents of each of the documents recited in this paragraph is herein incorporated by reference in its entirety.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of CTGF-4 polypeptides, studying conditions and/or disorders associated with aberrant CTGF-4 expression, and in screening for compounds effective in ameliorating such conditions and/or disorders.

### Example 31

### CTGF-4 Knock-Out Animals

Endogenous CTGF-4 gene expression can also be reduced by inactivating or “knocking out” the CTGF-4 gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the CTGF-4 polypeptides. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Knock-out animals of the invention have uses which include, but are not limited to, animal model systems useful in elaborating the biological function of CTGF-4 polypeptides, studying conditions and/or disorders associated with aberrant CTGF-4 expression, and in screening for compounds effective in ameliorating such conditions and/or disorders.

### Example 32

### Assays Detecting Stimulation or Inhibition of B cell Proliferation and Differentiation

Generation of functional humoral immune responses requires both soluble and cognate signaling between B-lineage cells and their microenvironment. Signals may impart a positive stimulus that allows a B-lineage cell to continue its programmed development, or a negative stimulus that instructs the cell to arrest its current developmental pathway. To date, numerous stimulatory and inhibitory signals have been found to influence B cell responsiveness including IL-2, IL-4, IL-5, IL-6, IL-7, IL10, IL-13, IL-14 and IL-15. Interestingly, these signals are by themselves weak effectors but can, in combination with various co-stimulatory proteins, induce activation, proliferation, differentiation, homing, tolerance and death among B cell populations.

One of the best studied classes of B-cell co-stimulatory proteins is the TNF-superfamily. Within this family CD40, CD27, and CD30 along with their respective ligands CD154, CD70, and CD153 have been found to regulate a variety of immune responses. Assays which allow for the detection and/or observation of the proliferation and differentiation of these B-cell populations and their precursors are valuable tools in determining the effects various proteins may have on these B-cell populations in terms of proliferation and differentiation. Listed below are two assays designed to allow for the detection of the differentiation, proliferation, or inhibition of B-cell populations and their precursors.

In Vitro Assay—Purified CTGF-4 protein, or truncated forms thereof, is assessed for its ability to induce activation, proliferation, differentiation or inhibition and/or death in B-cell populations and their precursors. The activity of CTGF-4 protein on purified human tonsillar B cells, measured qualitatively over the dose range from 0.1 to 10,000 ng/mL, is assessed in a standard B-lymphocyte co-stimulation assay in which purified tonsillar B cells are cultured in the presence of either formalin-fixed Staphylococcus aureus Cowan I (SAC) or immobilized anti-human IgM antibody as the priming agent. Second signals such as IL-2 and IL-15 synergize with SAC and IgM crosslinking to elicit B cell proliferation as measured by tritiated-thymidine incorporation. Novel synergizing agents can be readily identified using this assay. The assay involves isolating human tonsillar B cells by magnetic bead (MACS) depletion of CD3-positive cells. The resulting cell population is greater than 95% B cells as assessed by expression of CD45R(B220).

Various dilutions of each sample are placed into individual wells of a 96-well plate to which are added 10⁵ B-cells suspended in culture medium (RPMI 1640 containing 10% FBS, 5×10⁻⁵M 2ME, 100U/ml penicillin, 10 ug/ml streptomycin, and 10⁻⁵ dilution of SAC) in a total volume of 150 ul. Proliferation or inhibition is quantitated by a 20 h pulse (1 uCi/well) with 3H-thymidine (6.7 Ci/mM) beginning 72 h post factor addition. The positive and negative controls are IL2 and medium respectively.

In Vivo Assay—BALB/c mice are injected (i.p.) twice per day with buffer only, or 2 mg/Kg of CTGF-4 protein, or truncated forms thereof. Mice receive this treatment for 4 consecutive days, at which time they are sacrificed and various tissues and serum collected for analyses. Comparison of H&E sections from normal and CTGF-4 protein-treated spleens identify the results of the activity of CTGF-4 protein on spleen cells, such as the diffusion of peri-arterial lymphatic sheaths, and/or significant increases in the nucleated cellularity of the red pulp regions, which may indicate the activation of the differentiation and proliferation of B-cell populations. Immunohistochemical studies using a B cell marker, anti-CD45R(B220), are used to determine whether any physiological changes to splenic cells, such as splenic disorganization, are due to increased B-cell representation within loosely defined B-cell zones that infiltrate established T-cell regions.

Flow cytometric analyses of the spleens from CTGF-4 protein-treated mice is used to indicate whether CTGF-4 protein specifically increases the proportion of ThB+, CD45R(B220)dull B cells over that which is observed in control mice.

Likewise, a predicted consequence of increased mature B-cell representation in vivo is a relative increase in serum Ig titers. Accordingly, serum IgM and IgA levels are compared between buffer and CTGF-4 protein-treated mice.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 33

### T Cell Proliferation Assay

A CD3-induced proliferation assay is performed on PBMCs and is measured by the uptake of ³H-thymidine. The assay is performed as follows. Ninety-six well plates are coated with 100 microliters/well of mAb to CD3 (HIT3a, Pharmingen) or isotype-matched control mAb (B33.1) overnight at 4° C. (1 microgram/ml in 0.05M bicarbonate buffer, pH 9.5), then washed three times with PBS. PBMC are isolated by F/H gradient centrifugation from human peripheral blood and added to quadruplicate wells (5×10⁴/well) of mAb coated plates in RPMI containing 10% FCS and P/S in the presence of varying concentrations of CTGF-4 protein (total volume 200 microliters). Relevant protein buffer and medium alone are controls. After 48 hr. culture at 37° C., plates are spun for 2 min. at 1000 rpm and 100 microliters of supernatant is removed and stored −20° C. for measurement of IL-2 (or other cytokines) if effect on proliferation is observed. Wells are supplemented with 100 microliters of medium containing 0.5 microCuries of ³H-thymidine and cultured at 37° C. for 18-24 hr. Wells are harvested and incorporation of ³H-thymidine used as a measure of proliferation. Anti-CD3 alone is the positive control for proliferation. IL-2 (100 U/ml) is also used as a control which enhances proliferation. Control antibody which does not induce proliferation of T cells is used as the negative controls for the effects of CTGF-4 proteins.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 34

### Effect of CTGF-4 on the Expression of MHC Class II, Costimulatory and Adhesion Molecules and Cell Differentiation of Monocytes and Monocyte-Derived Human Dendritic Cells

Dendritic cells are generated by the expansion of proliferating precursors found in the peripheral blood: adherent PBMC or elutriated monocytic fractions are cultured for 7-10 days with GM-CSF (50 ng/ml) and IL-4 (20 ng/ml). These dendritic cells have the characteristic phenotype of immature cells (expression of CD1, CD80, CD86, CD40 and MHC class II antigens). Treatment with activating factors, such as TNF-alpha, causes a rapid change in surface phenotype (increased expression of MHC class I and II, costimulatory and adhesion molecules, downregulation of FCgammaRII, upregulation of CD83). These changes correlate with increased antigen-presenting capacity and with functional maturation of the dendritic cells.

FACS analysis of surface antigens is performed as follows. Cells are treated 1-3 days with increasing concentrations of CTGF-4 or LPS (positive control), washed with PBS containing 1% BSA and 0.02 mM sodium azide, and then incubated with 1:20 dilution of appropriate FITC- or PE-labeled monoclonal antibodies for 30 minutes at 4° C. After an additional wash, the labeled cells are analyzed by flow cytometry on a FACScan (Becton Dickinson).

Effect on the production of cytokines. Cytokines generated by dendritic cells, in particular IL-12, are important in the initiation of T-cell dependent immune responses. IL-12 strongly influences the development of Th1 helper T-cell immune response, and induces cytotoxic T and NK cell function. An ELISA is used to measure the IL-12 release as follows. Dendritic cells (10⁶/ml) are treated with increasing concentrations of CTGF-4 for 24 hours. LPS (100 ng/ml) is added to the cell culture as positive control. Supernatants from the cell cultures are then collected and analyzed for IL-12 content using commercial ELISA kit (e.g, R & D Systems (Minneapolis, Minn.)). The standard protocols provided with the kits are used.

Effect on the expression of MHC Class II, costimulatory and adhesion molecules. Three major families of cell surface antigens can be identified on monocytes: adhesion molecules, molecules involved in antigen presentation, and Fc receptor. Modulation of the expression of MHC class II antigens and other costimulatory molecules, such as B7 and ICAM-1, may result in changes in the antigen presenting capacity of monocytes and ability to induce T cell activation. Increase expression of Fe receptors may correlate with improved monocyte cytotoxic activity, cytokine release and phagocytosis.

FACS analysis is used to examine the surface antigens as follows. Monocytes are treated 1-5 days with increasing concentrations of CTGF-4 or LPS (positive control), washed with PBS containing 1% BSA and 0.02 mM sodium azide, and then incubated with 1:20 dilution of appropriate FITC- or PE-labeled monoclonal antibodies for 30 minutes at 4° C. After an additional wash, the labeled cells are analyzed by flow cytometry on a FACScan (Becton Dickinson).

Monocyte activation and/or increased survival. Assays for molecules that activate (or alternatively, inactivate) monocytes and/or increase monocyte survival (or alternatively, decrease monocyte survival) are known in the art and may routinely be applied to determine whether a molecule of the invention functions as an inhibitor or activator of monocytes. CTGF-4, agonists, or antagonists of CTGF-4 can be screened using the three assays described below. For each of these assays, Peripheral blood mononuclear cells (PBMC) are purified from single donor leukopacks (American Red Cross, Baltimore, Md.) by centrifugation through a Histopaque gradient (Sigma). Monocytes are isolated from PBMC by counterflow centrifugal elutriation.

1. Monocyte Survival Assay. Human peripheral blood monocytes progressively lose viability when cultured in absence of serum or other stimuli. Their death results from internally regulated process (apoptosis). Addition to the culture of activating factors, such as TNF-alpha dramatically improves cell survival and prevents DNA fragmentation. Propidium iodide (PI) staining is used to measure apoptosis as follows. Monocytes are cultured for 48 hours in polypropylene tubes in serum-free medium (positive control), in the presence of 100 ng/ml TNF-alpha (negative control), and in the presence of varying concentrations of the compound to be tested. Cells are suspended at a concentration of 2×10⁶/ml in PBS containing PI at a final concentration of 5 micrograms/ml, and then incubaed at room temperature for 5 minutes before FACScan analysis. PI uptake has been demonstrated to correlate with DNA fragmentation in this experimental paradigm.

2. Effect on cytokine release. An important function of monocytes/macrophages is their regulatory activity on other cellular populations of the immune system through the release of cytokines after stimulation. An ELISA to measure cytokine release is performed as follows. Human monocytes are incubated at a density of 5×10⁵ cells/ml with increasing concentrations of CTGF-4 and under the same conditions, but in the absence of CTGF-4. For EL-12 production, the cells are primed overnight with IFN (100 U/ml) in presence of CTGF-4. LPS (10 ng/ml) is then added. Conditioned media are collected after 24 h and kept frozen until use. Measurement of TNF-alpha, IL-10, MCP-1 and IL-8 is then performed using a commercially available ELISA kit (e.g, R & D Systems (Minneapolis, Minn.)) and applying the standard protocols provided with the kit.

3. Oxidative burst. Purified monocytes are plated in 96-w plate at 2-1×10⁵ cell/well. Increasing concentrations of CTGF-4 are added to the wells in a total volume of 0.2 ml culture medium (RPMI 1640+10% FCS, glutamine and antibiotics). After 3 days incubation, the plates are centrifuged and the medium is removed from the wells. To the macrophage monolayers, 0.2 ml per well of phenol red solution (140 mM NaCl, 10 mM potassium phosphate buffer pH 7.0, 5.5 mM dextrose, 0.56 mM phenol red and 19 U/ml of HRPO) is added, together with the stimulant (200 nM PMA). The plates are incubated at 37° C. for 2 hours and the reaction is stopped by adding 20 μl 1N NaOH per well. The absorbance is read at 610 nm. To calculate the amount of H₂O₂ produced by the macrophages, a standard curve of a H₂O₂ solution of known molarity is performed for each experiment.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 35

### CTGF-4 Biological Effects

Astrocyte and Neuronal Assays. Recombinant CTGF-4, expressed in Escherichia coli and purified as described above, can be tested for activity in promoting the survival, neurite outgrowth, or phenotypic differentiation of cortical neuronal cells and for inducing the proliferation of glial fibrillary acidic protein immunopositive cells, astrocytes. The selection of cortical cells for the bioassay is based on the prevalent expression of FGF-1 and FGF-2 in cortical structures and on the previously reported enhancement of cortical neuronal survival resulting from FGF-2 treatment. A thymidine incorporation assay, for example, can be used to elucidate CTGF-4's activity on these cells.

Moreover, previous reports describing the biological effects of FGF-2 (basic FGF) on cortical or hippocampal neurons in vitro have demonstrated increases in both neuron survival and neurite outgrowth (Walicke, P. et al., “Fibroblast growth factor promotes survival of dissociated hippocampal neurons and enhances neurite extension.” Proc. Natl. Acad. Sci. USA 83:3012-3016. (1986), assay herein incorporated by reference in its entirety). However, reports from experiments done on PC-12 cells suggest that these two responses are not necessarily synonymous and may depend on not only which FGF is being tested but also on which receptor(s) are expressed on the target cells. Using the primary cortical neuronal culture paradigm, the ability of CTGF-4 to induce neurite outgrowth can be compared to the response achieved with FGF-2 using, for example, a thymidine incorporation assay.

Fibroblast and endothelial cell assays. Human lung fibroblasts are obtained from Clonetics (San Diego, Calif.) and maintained in growth media from Clonetics. Dermal microvascular endothelial cells are obtained from Cell Applications (San Diego, Calif.). For proliferation assays, the human lung fibroblasts and dermal microvascular endothelial cells can be cultured at 5,000 cells/well in a 96-well plate for one day in growth medium. The cells are then incubated for one day in 0.1% BSA basal medium. After replacing the medium with fresh 0.1% BSA medium, the cells are incubated with the test proteins for 3 days. Alamar Blue (Alamar Biosciences, Sacramento, Calif.) is added to each well to a final concentration of 10%. The cells are incubated for 4 hr. Cell viability is measured by reading in a CytoFluor fluorescence reader. For the PGE₂ assays, the human lung fibroblasts are cultured at 5,000 cells/well in a 96-well plate for one day. After a medium change to 0.1% BSA basal medium, the cells are incubated with FGF-2 or CTGF-4 with or without IL-1α for 24 hours. The supernatants are collected and assayed for PGE₂ by EIA kit (Cayman, Ann Arbor, Mich.). For the IL-6 assays, the human lung fibroblasts are cultured at 5,000 cells/well in a 96-well plate for one day. After a medium change to 0.1% BSA basal medium, the cells are incubated with FGF-2 or CTGF-4 with or without IL-1α for 24 hours. The supernatants are collected and assayed for IL-6 by ELISA kit (Endogen, Cambridge, Mass.).

Human lung fibroblasts are cultured with FGF-2 or CTGF-4 for 3 days in basal medium before the addition of Alamar Blue to assess effects on growth of the fibroblasts. FGF-2 should show a stimulation at 10-2500 ng/ml which can be used to compare stimulation with CTGF-4.

Parkinson Models. The loss of motor function in Parkinson's disease is attributed to a deficiency of striatal dopamine resulting from the degeneration of the nigrostriatal dopaminergic projection neurons. An animal model for Parkinson's that has been extensively characterized involves the systemic administration of 1-methyl-4 phenyl 1,2,3,6-tetrahydropyridine (MPTP). In the CNS, MPTP is taken-up by astrocytes and catabolized by monoamine oxidase B to 1-methyl-4-phenyl pyridine (MPP⁺) and released. Subsequently, MPP⁺ is actively accumulated in dopaminergic neurons by the high-affinity reuptake transporter for dopamine. MPP⁺ is then concentrated in mitochondria by the electrochemical gradient and selectively inhibits nicotidamide adenine disphosphate: ubiquinone oxidoreductionase (complex I), thereby interfering with electron transport and eventually generating oxygen radicals.

It has been demonstrated in tissue culture paradigms that FGF-2 (basic FGF) has trophic activity towards nigral dopaminergic neurons (Ferrari et al., Dev. Biol. 1989). Recently, Dr. Unsicker's group has demonstrated that administering FGF-2 in gel foam implants in the striatum results in the near complete protection of nigral dopaminergic neurons from the toxicity associated with MPTP exposure (Otto and Unsicker, J. Neuroscience, 1990).

Based on the data with FGF-2, CTGF-4 can be evaluated to determine whether it has an action similar to that of FGF-2 in enhancing dopaminergic neuronal survival in vitro and it can also be tested in vivo for protection of dopaminergic neurons in the striatum from the damage associated with MPTP treatment. The potential effect of CTGF-4 is first examined in vitro in a dopaminergic neuronal cell culture paradigm. The cultures are prepared by dissecting the midbrain floor plate from gestation day 14 Wistar rat embryos. The tissue is dissociated with trypsin and seeded at a density of 200,000 cells/cm² on polyorthinine-laminin coated glass coverslips. The cells are maintained in Dulbecco's Modified Eagle's medium and F12 medium containing hormonal supplements (N1). The cultures are fixed with paraformaldehyde after 8 days in vitro and are processed for tyrosine hydroxylase, a specific marker for dopminergic neurons, immunohistochemical staining. Dissociated cell cultures are prepared from embryonic rats. The culture medium is changed every third day and the factors are also added at that time.

Since the dopaminergic neurons are isolated from animals at gestation day 14, a developmental time which is past the stage when the dopaminergic precursor cells are proliferating, an increase in the number of tyrosine hydroxylase immunopositive neurons would represent an increase in the number of dopaminergic neurons surviving in vitro. Therefore, if CTGF-4 acts to prolong the survival of dopaminergic neurons, it would suggest that CTGF-4 may be involved in Parkinson's Disease.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 36

### The Effect of CTGF-4 on the Growth of Vascular Endothelial Cells

On day 1, human umbilical vein endothelial cells (HUVEC) are seeded at 2-5×10⁴ cells/35 mm dish density in M199 medium containing 4% fetal bovine serum (FBS), 16 units/ml heparin, and 50 units/ml endothelial cell growth supplements (ECGS, Biotechnique, Inc.). On day 2, the medium is replaced with M199 containing 10% FBS, 8 units/ml heparin. CTGF-4 protein of SEQ ID NO. 2, and positive controls, such as VEGF and basic FGF (bFGF) are added, at varying concentrations. On days 4 and 6, the medium is replaced. On day 8, cell number is determined with a Coulter Counter.

An increase in the number of HUVEC cells indicates that CTGF-4 may proliferate vascular endothelial cells.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 37

### Stimulatory Effect of CTGF-4 on the Proliferation of Vascular Endothelial Cells

For evaluation of mitogenic activity of growth factors, the colorimetric MTS (3-(4,5-dimethylthiazol-2-yl)-5-(3-carboxymethoxyphenyl)-2-(4-sulfophenyl)₂H-tetrazolium) assay with the electron coupling reagent PMS (phenazine methosulfate) was performed (CellTiter 96 AQ, Promega). Cells are seeded in a 96-well plate (5,000 cells/well) in 0.1 mL serum-supplemented medium and are allowed to attach overnight. After serum-starvation for 12 hours in 0.5%FBS, conditions (bFGF, VEGF₁₆₅ or CTGF-4 in 0.5% FBS) with or without Heparin (8 U/ml) are added to wells for 48 hours. 20 mg of MTS/PMS mixture (1:0.05) are added per well and allowed to incubate for 1 hour at 37° C. before measuring the absorbance at 490 nm in an ELISA plate reader. Background absorbance from control wells (some media, no cells) is subtracted, and seven wells are performed in parallel for each condition. See, Leak et al. In Vitro Cell. Dev. Biol. 30A:512-518 (1994).

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 38

### Inhibition of PDGF-Induced Vascular Smooth Muscle Cell Proliferation Stimulatory Effect

HAoSMC proliferation can be measured, for example, by BrdUrd incorporation. Briefly, subconfluent, quiescent cells grown on the 4-chamber slides are transfected with CRP or FITC-labeled AT2-3LP. Then, the cells are pulsed with 10% calf serum and 6 mg/ml BrdUrd. After 24 h, immunocytochemistry is performed by using BrdUrd Staining Kit (Zymed Laboratories). In brief, the cells are incubated with the biotinylated mouse anti-BrdUrd antibody at 4° C. for 2 h after being exposed to denaturing solution and then incubated with the streptavidin-peroxidase and diaminobenzidine. After counterstaining with hematoxylin, the cells are mounted for microscopic examination, and the BrdUrd-positive cells are counted. The BrdUrd index is calculated as a percent of the BrdUrd-positive cells to the total cell number. In addition, the simultaneous detection of the BrdUrd staining (nucleus) and the FITC uptake (cytoplasm) is performed for individual cells by the concomitant use of bright field illumination and dark field-UV fluorescent illumination. See, Hayashida et al., J. Biol. Chem. 6:271(36):21985-21992 (1996).

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 39

### Stimulation of Endothelial Migration

This example will be used to explore the possibility that CTGF-4 may stimulate lymphatic endothelial cell migration.

Endothelial cell migration assays are performed using a 48 well microchemotaxis chamber (Neuroprobe Inc., Cabin John, MD; Falk, W., et al., J. Immunological Methods 1980;33:239-247). Polyvinylpyrrolidone-free polycarbonate filters with a pore size of 8 um (Nucleopore Corp. Cambridge, Mass.) are coated with 0.1% gelatin for at least 6 hours at room temperature and dried under sterile air. Test substances are diluted to appropriate concentrations in M199 supplemented with 0.25% bovine serum albumin (BSA), and 25 ul of the final dilution is placed in the lower chamber of the modified Boyden apparatus. Subconfluent, early passage (2-6) HUVEC or BMEC cultures are washed and trypsinized for the minimum time required to achieve cell detachment. After placing the filter between lower and upper chamber, 2.5×10⁵ cells suspended in 50 ul M199 containing 1% FBS are seeded in the upper compartment. The apparatus is then incubated for 5 hours at 37° C. in a humidified chamber with 5% CO₂ to allow cell migration. After the incubation period, the filter is removed and the upper side of the filter with the non-migrated cells is scraped with a rubber policeman. The filters are fixed with methanol and stained with a Giemsa solution (Diff-Quick, Baxter, McGraw Park, Ill.). Migration is quantified by counting cells of three random high-power fields (40×) in each well, and all groups are performed in quadruplicate.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 40

### Stimulation of Nitric Oxide Production by Endothelial Cells

Nitric oxide released by the vascular endothelium is believed to be a mediator of vascular endothelium relaxation. Thus, CTGF-4 activity can be assayed by determining nitric oxide production by endothelial cells in response to CTGF-4.

Nitric oxide is measured in 96-well plates of confluent microvascular endothelial cells after 24 hours starvation and a subsequent 4 hr exposure to various levels of a positive control (such as VEGF-1) and CTGF-4. Nitric oxide in the medium is determined by use of the Griess reagent to measure total nitrite after reduction of nitric oxide-derived nitrate by nitrate reductase. The effect of CTGF-4 on nitric oxide release is examined on HUVEC.

Briefly, NO release from cultured HUVEC monolayer is measured with a NO-specific polarographic electrode connected to a NO meter (Iso-NO, World Precision Instruments Inc.) (1049). Calibration of the NO elements is performed according to the following equation:

2KNO₂+2KI+2H₂SO₄62NO+I₂+2H₂O+2K₂SO₄

The standard calibration curve is obtained by adding graded concentrations of KNO₂ (0, 5, 10, 25, 50, 100, 250, and 500 mmol/L) into the calibration solution containing KI and H₂SO₄. The specificity of the Iso-NO electrode to NO is previously determined by measurement of NO from authentic NO gas (1050). The culture medium is removed and HUVECs are washed twice with Dulbecco's phosphate buffered saline. The cells are then bathed in 5 ml of filtered Krebs-Henseleit solution in 6-well plates, and the cell plates are kept on a slide warmer (Lab Line Instruments Inc.) To maintain the temperature at 37° C. The NO sensor probe is inserted vertically into the wells, keeping the tip of the electrode 2 mm under the surface of the solution, before addition of the different conditions. S-nitroso acetyl penicillamin (SNAP) is used as a positive control. The amount of released NO is expressed as picomoles per 1×10⁶ endothelial cells. All values reported are means of four to six measurements in each group (number of cell culture wells). See, Leak et al. Biochem. and Biophys. Res. Comm. 217:96-105 (1995).

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 41

### Effect of CTGF-4 on Cord Formation in Angiogenesis

Another step in angiogenesis is cord formation, marked by differentiation of endothelial cells. This bioassay measures the ability of microvascular endothelial cells to form capillary-like structures (hollow structures) when cultured in vitro.

CADMEC (microvascular endothelial cells) are purchased from Cell Applications, Inc. as proliferating (passage 2) cells and are cultured in Cell Applications' CADMEC Growth Medium and used at passage 5. For the in vitro angiogenesis assay, the wells of a 48-well cell culture plate are coated with Cell Applications' Attachment Factor Medium (200 ml/well) for 30 min. at 37° C. CADMEC are seeded onto the coated wells at 7,500 cells/well and cultured overnight in Growth Medium. The Growth Medium is then replaced with 300 mg Cell Applications Chord Formation Medium containing control buffer or CTGF-4 (0.1 to 100 ng/ml) and the cells are cultured for an additional 48 hr. The numbers and lengths of the capillary-like chords are quantitated through use of the Boeckeler VIA-170 video image analyzer. All assays are done in triplicate.

Commercial (R&D) VEGF (50 ng/ml) is used as a positive control. b-esteradiol (1 ng/ml) is used as a negative control. The appropriate buffer (without protein) is also utilized as a control.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 42

### Angiogenic Effect on Chick Chorioallantoic Membrane

Chick chorioallantoic membrane (CAM) is a well-established system to examine angiogenesis. Blood vessel formation on CAM is easily visible and quantifiable. The ability of CTGF-4 to stimulate angiogenesis in CAM can be examined.

Fertilized eggs of the White Leghorn chick (Gallus gallus) and the Japanese qual (Coturnix coturnix) are incubated at 37.8° C. and 80% humidity. Differentiated CAM of 16-day-old chick and 13-day-old qual embryos is studied with the following methods.

On Day 4 of development, a window is made into the egg shell of chick eggs. The embryos are checked for normal development and the eggs sealed with cellotape. They are further incubated until Day 13. Thermanox coverslips (Nunc, Naperville, Ill.) are cut into disks of about 5 mm in diameter. Sterile and salt-free growth factors are dissolved in distilled water and about 3.3 mg/5 ml are pipetted on the disks. After air-drying, the inverted disks are applied on CAM. After 3 days, the specimens are fixed in 3% glutaraldehyde and 2% formaldehyde and rinsed in 0.12 M sodium cacodylate buffer. They are photographed with a stereo microscope [Wild M8] and embedded for semi- and ultrathin sectioning as described above. Controls are performed with carrier disks alone.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 43

### Angiogenesis Assay Using a Matrigel Implant in Mouse

In vivo angiogenesis assay of CTGF-4 measures the ability of an existing capillary network to form new vessels in an implanted capsule of murine extracellular matrix material (Matrigel). The protein is mixed with the liquid Matrigel at 4° C. and the mixture is then injected subcutaneously in mice where it solidifies. After 7 days, the solid “plug” of Matrigel is removed and examined for the presence of new blood vessels. Matrigel is purchased from Becton Dickinson Labware/Collaborative Biomedical Products.

When thawed at 4° C. the Matrigel material is a liquid. The Matrigel is mixed with CTGF-4 at 150 ng/ml at 4 degree C. and drawn into cold 3 ml syringes. Female C57Bl/6 mice approximately 8 weeks old are injected with the mixture of Matrigel and experimental protein at 2 sites at the midventral aspect of the abdomen (0.5 ml/site). After 7 days, the mice are sacrificed by cervical dislocation, the Matrigel plugs are removed and cleaned (i.e., all clinging membranes and fibrous tissue is removed). Replicate whole plugs are fixed in neutral buffered 10% formaldehyde, embedded in paraffin and used to produce sections for histological examination after staining with Masson's Trichrome. Cross sections from 3 different regions of each plug are processed. Selected sections are stained for the presence of vWF. The positive control for this assay is bovine basic FGF (150 ng/ml). Matrigel alone is used to determine basal levels of angiogenesis.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 44

### Rescue of Ischemia in Rabbit Lower Limb Model

To study the in vivo effects of CTGF-4 on ischemia, a rabbit hindlimb ischemia model is created by surgical removal of one femoral arteries as described previously (Takeshita, S. et al., Am J. Pathol 147:1649-1660 (1995)). The excision of the femoral artery results in retrograde propagation of thrombus and occlusion of the external iliac artery. Consequently, blood flow to the ischermic limb is dependent upon collateral vessels originating from the internal iliac artery (Takeshita, S. et al. Am J. Pathol 147:1649-1660 (1995)). An interval of 10 days is allowed for post-operative recovery of rabbits and development of endogenous collateral vessels. At 10 day post-operatively (day 0), after performing a baseline angiogram, the internal iliac artery of the ischemic limb is transfected with 500 mg naked CTGF-4 expression plasmid by arterial gene transfer technology using a hydrogel-coated balloon catheter as described (Riessen, R. et al. Hum Gene Ther. 4:749-758 (1993); Leclerc, G. et al. J. Clin. Invest. 90: 936-944 (1992)). When CTGF-4 is used in the treatment, a single bolus of 500 mg CTGF-4 protein or control is delivered into the internal iliac artery of the ischemic limb over a period of 1 min. through an infusion catheter. On day 30, various parameters are measured in these rabbits: (a) BP ratio—The blood pressure ratio of systolic pressure of the ischemic limb to that of normal limb; (b) Blood Flow and Flow Reserve—Resting FL: the blood flow during undilated condition and Max FL: the blood flow during fully dilated condition (also an indirect measure of the blood vessel amount) and Flow Reserve is reflected by the ratio of max FL: resting FL; (c) Angiographic Score—This is measured by the angiogram of collateral vessels. A score is determined by the percentage of circles in an overlaying grid that with crossing opacified arteries divided by the total number m the rabbit thigh; (d) Capillary density—The number of collateral capillaries determined in light microscopic sections taken from hindlimbs.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 45

### Effect of CTGF-4 on Vasodilation

Since dilation of vascular endothelium is important in reducing blood pressure, the ability of CTGF-4 to affect the blood pressure in spontaneously hypertensive rats (SHR) is examined. Increasing doses (0, 10, 30, 100, 300, and 900 mg/kg) of the CTGF-4 are administered to 13-14 week old spontaneously hypertensive rats (SHR). Data are expressed as the mean+/−SEM. Statistical analysis are performed with a paired t-test and statistical significance is defined as p<0.05 vs. the response to buffer alone.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 46

### Rat Ischemic Skin Flap Model

The evaluation parameters include skin blood flow, skin temperature, and factor VIII immunohistochemistry or endothelial alkaline phosphatase reaction. CTGF-4 expression, during the skin ischemia, is studied using in situ hybridization.

The study in this model is divided into three parts as follows:

a) Ischemic skin

b) Ischemic skin wounds

c) Normal wounds

The experimental protocol includes:

a) Raising a 3×4 cm, single pedicle full-thickness random skin flap (myocutaneous flap over the lower back of the animal).

b) An excisional wounding (4-6 mm in diameter) in the ischemic skin (skin-flap).

c) Topical treatment with CTGF-4 of the excisional wounds (day 0, 1, 2, 3, 4 post-wounding) at the following various dosage ranges: 1 mg to 100 mg.

d) Harvesting the wound tissues at day 3, 5, 7, 10, 14 and 21 post-wounding for histological, immunohistochemical, and in situ studies.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 47

### Peripheral Arterial Disease Model

Angiogenic therapy using CTGF-4 is a novel therapeutic strategy to obtain restoration of blood flow around the ischemia in case of peripheral arterial diseases. The experimental protocol includes:

a) One side of the femoral artery is ligated to create ischemic muscle of the hindlimb, the other side of hindlimb serves as a control.

b) CTGF-4 protein, in a dosage range of 20 mg-500 mg, is delivered intravenously and/or intramuscularly 3 times (perhaps more) per week for 2-3 weeks.

c) The ischemic muscle tissue is collected after ligation of the femoral artery at 1, 2, and 3 weeks for the analysis of CTGF-4 expression and histology. Biopsy is also performed on the other side of normal muscle of the contralateral hindlimb.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 48

### Ischemic Myocardial Disease Model

CTGF-4 is evaluated as a potent mitogen capable of stimulating the development of collateral vessels, and restructuring new vessels after coronary artery occlusion. Alteration of CTGF-4 expression is investigated in situ. The experimental protocol includes:

a) The heart is exposed through a left-side thoracotomy in the rat. Immediately, the left coronary artery is occluded with a thin suture (6-0) and the thorax is closed.

b) CTGF-4 protein, in a dosage range of 20 mg-500 mg, is delivered intravenously and/or intramuscularly 3 times (perhaps more) per week for 2-4 weeks.

c) Thirty days after the surgery, the heart is removed and cross-sectioned for morphometric and in situ analyzes.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 49

### Rat Corneal Wound Healing Model

This animal model shows the effect of CTGF-4 on neovascularization. The experimental protocol includes:

a) Making a 1-1.5 mm long incision from the center of cornea into the stromal layer.

b) Inserting a spatula below the lip of the incision facing the outer corner of the eye.

c) Making a pocket (its base is 1-1.5 mm form the edge of the eye).

d) Positioning a pellet, containing 50 ng-5 ug of CTGF-4, within the pocket.

e) CTGF-4 treatment can also be applied topically to the corneal wounds in a dosage range of 20 mg-500 mg (daily treatment for five days).

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 50

### Diabetic Mouse and Glucocorticoid-Impaired Wound Healing Models

A. Diabetic db+/db+ Mouse Model.

To demonstrate that CTGF-4 accelerates the healing process, the genetically diabetic mouse model of wound healing is used. The full thickness wound healing model in the db+/db+ mouse is a well characterized, clinically relevant and reproducible model of impaired wound healing. Healing of the diabetic wound is dependent on formation of granulation tissue and re-epithelialization rather than contraction (Gartner, M. H. et al., J. Surg. Res. 52:389 (1992); Greenhalgh, D. G. et al., Am. J. Pathol. 136:1235 (1990)).

The diabetic animals have many of the characteristic features observed in Type II diabetes mellitus. Homozygous (db+/db+) mice are obese in comparison to their normal heterozygous (db+/+m) littermates. Mutant diabetic (db+/db+) mice have a single autosomal recessive mutation on chromosome 4 (db+)(Coleman et al. Proc. Natl. Acad. Sci. USA 77:283-293 (1982)). Animals show polyphagia, polydipsia and polyuria. Mutant diabetic mice (db+/db+) have elevated blood glucose, increased or normal insulin levels, and suppressed cell-mediated immunity (Mandel et al., J. Immunol. 120:1375 (1978); Debray-Sachs, M. et al., Clin. Exp. Immunol. 51(1):1-7 (1983); Leiter et al., Am. J. of Pathol. 114:46-55 (1985)). Peripheral neuropathy, myocardial complications, and microvascular lesions, basement membrane thickening and glomerular filtration abnormalities have been described in these animals (Norido, F. et al., Exp. Neurol. 83(2):221-232 (1984); Robertson et al., Diabetes 29(1):60-67 (1980); Giacomelli et al., Lab Invest. 40(4):460-473 (1979); Coleman, D. L., Diabetes 31 (Suppl):1-6 (1982)). These homozygous diabetic mice develop hyperglycemia that is resistant to insulin analogous to human type II diabetes (Mandel et al., J. Immunol. 120:1375-1377 (1978)).

The characteristics observed in these animals suggests that healing in this model may be similar to the healing observed in human diabetes (Greenhalgh, et al., Am. J. of Pathol. 136:1235-1246 (1990)).

Genetically diabetic female C57BL/KsJ (db+/db+) mice and their non-diabetic (db+/+m) heterozygous littermates are used in this study (Jackson Laboratories). The animals are purchased at 6 weeks of age and are 8 weeks old at the beginning of the study. Animals are individually housed and received food and water ad libitum. All manipulations are performed using aseptic techniques. The experiments are conducted according to the rules and guidelines of Human Genome Sciences, Inc. Institutional Animal Care and Use Committee and the Guidelines for the Care and Use of Laboratory Animals.

Wounding protocol is performed according to previously reported methods (Tsuboi, R. and Rifkin, D. B., J. Exp. Med. 172:245-251 (1990)). Briefly, on the day of wounding, animals are anesthetized with an intraperitoneal injection of Avertin (0.01 mg/mL), 2,2,2-tribromoethanol and 2-methyl-2-butanol dissolved in deionized water. The dorsal region of the animal is shaved and the skin washed with 70% ethanol solution and iodine. The surgical area is dried with sterile gauze prior to wounding. An 8 mm full-thickness wound is then created using a Keyes tissue punch. Immediately following wounding, the surrounding skin is gently stretched to eliminate wound expansion. The wounds are left open for the duration of the experiment. Application of the treatment is given topically for 5 consecutive days commencing on the day of wounding. Prior to treatment, wounds are gently cleansed with sterile saline and gauze sponges.

Wounds are visually examined and photographed at a fixed distance at the day of surgery and at two day intervals thereafter. Wound closure is determined by daily measurement on days 1-5 and on day 8. Wounds are measured horizontally and vertically using a calibrated Jameson caliper. Wounds are considered healed if granulation tissue is no longer visible and the wound is covered by a continuous epithelium.

CTGF-4 is administered using at a range different doses of CTGF-4, from 4 mg to 500 mg per wound per day for 8 days in vehicle. Vehicle control groups received 50 mL of vehicle solution.

Animals are euthanized on day 8 with an intraperitoneal injection of sodium pentobarbital (300 mg/kg). The wounds and surrounding skin are then harvested for histology and immunohistochemistry. Tissue specimens are placed in 10% neutral buffered formalin in tissue cassettes between biopsy sponges for further processing.

Three groups of 10 animals each (5 diabetic and 5 non-diabetic controls) are evaluated: 1) Vehicle placebo control, 2) CTGF-4.

Wound closure is analyzed by measuring the area in the vertical and horizontal axis and obtaining the total square area of the wound. Contraction is then estimated by establishing the differences between the initial wound area (day 0) and that of post treatment (day 8). The wound area on day 1 is 64 mm², the corresponding size of the dermal punch. Calculations are made using the following formula:

[Open area on day 8]-[Open area on day 1]/[Open area on day 1]

Specimens are fixed in 10% buffered formalin and paraffin embedded blocks are sectioned perpendicular to the wound surface (5 mm) and cut using a Reichert-Jung microtome. Routine hematoxylin-eosin (H&E) staining is performed on cross-sections of bisected wounds. Histologic examination of the wounds are used to assess whether the healing process and the morphologic appearance of the repaired skin is altered by treatment with CTGF-4. This assessment included verification of the presence of cell accumulation, inflammatory cells, capillaries, fibroblasts, re-epithelialization and epidermal maturity (Greenhalgh, D. G. et al., Am. J. Pathol. 136:1235 (1990)). A calibrated lens micrometer is used by a blinded observer.

Tissue sections are also stained immunohistochemically with a polyclonal rabbit anti-human keratin antibody using ABC Elite detection system. Human skin is used as a positive tissue control while non-immune IgG is used as a negative control. Keratinocyte growth is determined by evaluating the extent of reepithelialization of the wound using a calibrated lens micrometer.

Proliferating cell nuclear antigen/cyclin (PCNA) in skin specimens is demonstrated by using anti-PCNA antibody (1:50) with an ABC Elite detection system. Human colon cancer served as a positive tissue control and human brain tissue is used as a negative tissue control. Each specimen included a section with omission of the primary antibody and substitution with non-immune mouse IgG. Ranking of these sections is based on the extent of proliferation on a scale of 0-8, the lower side of the scale reflecting slight proliferation to the higher side reflecting intense proliferation.

Experimental data are analyzed using an unpaired t test. A p value of <0.05 is considered significant.

B. Steroid Impaired Rat Model

The inhibition of wound healing by steroids has been well documented in various in vitro and in vivo systems (Wahl, S. M. Glucocorticoids and Wound healing. In: Anti-Inflammatory Steroid Action: Basic and Clinical Aspects. 280-302 (1989); Wahl, S. M. et al., J. Immunol. 115: 476-481 (1975); Werb, Z. et al., J. Exp. Med. 147:1684-1694 (1978)). Glucocorticoids retard wound healing by inhibiting angiogenesis, decreasing vascular permeability (Ebert, R. H., et al., An. Intern. Med. 37:701-705 (1952)), fibroblast proliferation, and collagen synthesis (Beck, L. S. et al., Growth Factors. 5: 295-304 (1991); Haynes, B. F. et al., J. Clin. Invest. 61: 703-797 (1978)) and producing a transient reduction of circulating monocytes (Haynes, B. F., et al., J. Clin. Invest. 61: 703-797 (1978); Wahl, S. M., “Glucocorticoids and wound healing”, In: Antiinflammatory Steroid Action: Basic and Clinical Aspects, Academic Press, New York, pp. 280-302 (1989)). The systemic administration of steroids to impaired wound healing is a well establish phenomenon in rats (Beck, L. S. et al., Growth Factors. 5: 295-304 (1991); Haynes, B. F., et al., J. Clin. Invest. 61: 703-797 (1978); Wahl, S. M., “Glucocorticoids and wound healing”, In: Antuinflammatory Steroid Action: Basic and Clinical Aspects, Academic Press, New York, pp. 280-302 (1989); Pierce, G. F. et al., Proc. Natl. Acad. Sci. USA 86: 2229-2233 (1989)).

To demonstrate that CTGF-4 can accelerate the healing process, the effects of multiple topical applications of CTGF-4 on full thickness excisional skin wounds in rats in which healing has been impaired by the systemic administration of methylprednisolone is assessed.

Young adult male Sprague Dawley rats weighing 250-300 g (Charles River Laboratories) are used in this example. The animals are purchased at 8 weeks of age and are 9 weeks old at the beginning of the study. The healing response of rats is impaired by the systemic administration of methylprednisolone (17 mg/kg/rat intramuscularly) at the time of wounding. Animals are individually housed and received food and water ad libitum. All manipulations are performed using aseptic techniques. This study is conducted according to the rules and guidelines of Human Genome Sciences, Inc. Institutional Animal Care and Use Committee and the Guidelines for the Care and Use of Laboratory Animals.

The wounding protocol is followed according to section A, above. On the day of wounding, animals are anesthetized with an intramuscular injection of ketamine (50 mg/kg) and xylazine (5 mg/kg). The dorsal region of the animal is shaved and the skin washed with 70% ethanol and iodine solutions. The surgical area is dried with sterile gauze prior to wounding. An 8 mm full-thickness wound is created using a Keyes tissue punch. The wounds are left open for the duration of the experiment. Applications of the testing materials are given topically once a day for 7 consecutive days commencing on the day of wounding and subsequent to methylprednisolone administration. Prior to treatment, wounds are gently cleansed with sterile saline and gauze sponges.

Wounds are visually examined and photographed at a fixed distance at the day of wounding and at the end of treatment. Wound closure is determined by daily measurement on days 1-5 and on day 8. Wounds are measured horizontally and vertically using a calibrated Jameson caliper. Wounds are considered healed if granulation tissue is no longer visible and the wound is covered by a continuous epithelium.

CTGF-4 is administered using at a range different doses of CTGF-4, from 4 mg to 500 mg per wound per day for 8 days in vehicle. Vehicle control groups received 50 mL of vehicle solution.

Animals are euthanized on day 8 with an intraperitoneal injection of sodium pentobarbital (300 mg/kg). The wounds and surrounding skin are then harvested for histology. Tissue specimens are placed in 10% neutral buffered formalin in tissue cassettes between biopsy sponges for further processing.

Four groups of 10 animals each (5 with methylprednisolone and 5 without glucocorticoid) are evaluated: 1) Untreated group 2) Vehicle placebo control 3) CTGF-4 treated groups.

Wound closure is analyzed by measuring the area in the vertical and horizontal axis and obtaining the total area of the wound. Closure is then estimated by establishing the differences between the initial wound area (day 0) and that of post treatment (day 8). The wound area on day 1 is 64 mm², the corresponding size of the dermal punch. Calculations are made using the following formula:

[Open area on day 8]-[Open area on day 1]/[Open area on day 1]

Specimens are fixed in 10% buffered formalin and paraffin embedded blocks are sectioned perpendicular to the wound surface (5 mm) and cut using an Olympus microtome. Routine hematoxylin-eosin (H&E) staining is performed on cross-sections of bisected wounds. Histologic examination of the wounds allows assessment of whether the healing process and the morphologic appearance of the repaired skin is improved by treatment with CTGF-4. A calibrated lens micrometer is used by a blinded observer to determine the distance of the wound gap.

Experimental data are analyzed using an unpaired t test. A p value of <0.05 is considered significant.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 51

### Lymphadema Animal Model

The purpose of this experimental approach is to create an appropriate and consistent lymphedema model for testing the therapeutic effects of CTGF-4 in lymphangiogenesis and re-establishment of the lymphatic circulatory system in the rat hind limb. Effectiveness is measured by swelling volume of the affected limb, quantification of the amount of lymphatic vasculature, total blood plasma protein, and histopathology. Acute lymphedema is observed for 7-10 days. Perhaps more importantly, the chronic progress of the edema is followed for up to 3-4 weeks.

Prior to beginning surgery, blood sample is drawn for protein concentration analysis. Male rats weighing approximately ˜350 g are dosed with Pentobarbital. Subsequently, the right legs are shaved from knee to hip. The shaved area is swabbed with gauze soaked in 70% EtOH. Blood is drawn for serum total protein testing. Circumference and volumetric measurements are made prior to injecting dye into paws after marking 2 measurement levels (0.5 cm above heel, at mid-pt of dorsal paw). The intradermal dorsum of both right and left paws are injected with 0.05 ml of 1% Evan's Blue. Circumference and volumetric measurements are then made following injection of dye into paws.

Using the knee joint as a landmark, a mid-leg inguinal incision is made circumferentially allowing the femoral vessels to be located. Forceps and hemostats are used to dissect and separate the skin flaps. After locating the femoral vessels, the lymphatic vessel that runs along side and underneath the vessel(s) is located. The main lymphatic vessels in this area are then electrically coagulated or suture ligated.

Using a microscope, muscles in back of the leg (near the semitendinosis and adductors) are bluntly dissected. The popliteal lymph node is then located. The 2 proximal and 2 distal lymphatic vessels and distal blood supply of the popliteal node are then and ligated by suturing. The popliteal lymph node, and any accompanying adipose tissue, is then removed by cutting connective tissues.

Care is taken to control any mild bleeding resulting from this procedure. After lymphatics are occluded, the skin flaps are sealed by using liquid skin (Vetbond) (AJ Buck). The separated skin edges are sealed to the underlying muscle tissue while leaving a gap of ˜0.5 cm around the leg. Skin also may be anchored by suturing to underlying muscle when necessary.

To avoid infection, animals are housed individually with mesh (no bedding). Recovering animals are checked daily through the optimal edematous peak, which typically occurred by day 5-7. The plateau edematous peak are then observed. To evaluate the intensity of the lymphedema, the circumference and volumes of 2 designated places on each paw before operation and daily for 7 days are measured. The effect plasma proteins on lymphedema is determined and whether protein analysis is a useful testing perimeter is also investigated. The weights of both control and edematous limbs are evaluated at 2 places. Analysis is performed in a blind manner.

Circumference Measurements: Under brief gas anesthetic to prevent limb movement, a cloth tape is used to measure limb circumference. Measurements are done at the ankle bone and dorsal paw by 2 different people then those 2 readings are averaged. Readings are taken from both control and edematous limbs.

Volumetric Measurements: On the day of surgery, animals are anesthetized with Pentobarbital and are tested prior to surgery. For daily volumetrics animals are under brief halothane anesthetic (rapid immobilization and quick recovery), both legs are shaved and equally marked using waterproof marker on legs. Legs are first dipped in water, then dipped into instrument to each marked level then measured by Buxco edema software(Chen/Victor). Data is recorded by one person, while the other is dipping the limb to marked area.

Blood-plasma protein measurements: Blood is drawn, spun, and serum separated prior to surgery and then at conclusion for total protein and Ca2+ comparison.

Limb Weight Comparison: After drawing blood, the animal is prepared for tissue collection. The limbs are amputated using a quillitine, then both experimental and control legs are cut at the ligature and weighed. A second weighing is done as the tibio-cacaneal joint is disarticulated and the foot is weighed.

Histological Preparations: The transverse muscle located behind the knee (popliteal) area is dissected and arranged in a metal mold, filled with freezeGel, dipped into cold methylbutane, placed into labeled sample bags at −80° C. until sectioning. Upon sectioning, the muscle is observed under fluorescent microscopy for lymphatics.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

### Example 52

### Suppression of TNF Alpha-Induced Adhesion Molecule Expression by CTGF-4

The recruitment of lymphocytes to areas of inflammation and angiogenesis involves specific receptor-ligand interactions between cell surface adhesion molecules (CAMs) on lymphocytes and the vascular endothelium. The adhesion process, in both normal and pathological settings, follows a multi-step cascade that involves intercellular adhesion molecule-1 (ICAM-1), vascular cell adhesion molecule-1 (VCAM-1), and endothelial leukocyte adhesion molecule-1 (E-selectin) expression on endothelial cells (EC). The expression of these molecules and others on the vascular endothelium determines the efficiency with which leukocytes may adhere to the local vasculature and extravasate into the local tissue during the development of an inflammatory response. The local concentration of cytokines and growth factor participate in the modulation of the expression of these CAMs.

Tumor necrosis factor alpha (TNF-alpha), a potent proinflammatory cytokine, is a stimulator of all three CAMs on endothelial cells and may be involved in a wide variety of inflammatory responses, often resulting in a pathological outcome.

The potential of CTGF-4 to mediate a suppression of TNF-alpha induced CAM expression can be examined. A modified ELISA assay which uses ECs as a solid phase absorbent is employed to measure the amount of CAM expression on TNF-alpha treated ECs when co-stimulated with a member of the FGF family of proteins.

To perform the experiment, human umbilical vein endothelial cell (HUVEC) cultures are obtained from pooled cord harvests and maintained in growth medium (EGM-2; Clonetics, San Diego, Calif.) supplemented with 10% FCS and 1% penicillin/streptomycin in a 37 degree C. humidified incubator containing 5% CO₂. HUVECs are seeded in 96-well plates at concentrations of 1×10⁴ cells/well in EGM medium at 37° C. for 18-24 hrs or until confluent. The monolayers are subsequently washed 3 times with a serum-free solution of RPMI-1640 supplemented with 100 U/ml penicillin and 100 mg/ml streptomycin, and treated with a given cytokine and/or growth factor(s) for 24 h at 37° C. Following incubation, the cells are then evaluated for CAM expression.

Human Umbilical Vein Endothelial cells (HUVECs) are grown in a standard 96 well plate to confluence. Growth medium is removed from the cells and replaced with 90 ul of 199 Medium (10% FBS). Samples for testing and positive or negative controls are added to the plate in triplicate (in 10 ul volumes). Plates are incubated at 37° C. for either 5 h (selectin and integrin expression) or 24 h (integrin expression only). Plates are aspirated to remove medium and 100 μl of 0.1% paraformaldehyde-PBS(with Ca++ and Mg++) is added to each well. Plates are held at 4° C. for 30 min.

Fixative is then removed from the wells and wells are washed 1× with PBS(+Ca,Mg)+0.5% BSA and drained. Do not allow the wells to dry. Add 10 μl of diluted primary antibody to the test and control wells. Anti-ICAM-1-Biotin, Anti-ICAM-1-Biotin and Anti-E-selectin-Biotin are used at a concentration of 10 μg/ml (1:10 dilution of 0.1 mg/ml stock antibody). Cells are incubated at 37° C. for 30 min. in a humidified environment. Wells are washed ×3 with PBS(+Ca,Mg)+0.5% BSA.

Then add 20 μl of diluted ExtrAvidin-Alkaline Phosphotase (1:5,000 dilution) to each well and incubated at 37° C. for 30 min. Wells are washed X3 with PBS(+Ca,Mg)+0.5% BSA. 1 tablet of p-Nitrophenol Phosphate pNPP is dissolved in 5 ml of glycine buffer (pH 10.4). 100 μl of pNPP substrate in glycine buffer is added to each test well. Standard wells in triplicate are prepared from the working dilution of the ExtrAvidin-Alkaline Phosphotase in glycine buffer: 1:5,000 (10⁰)>10^{-0.5}>10⁻¹>10^{-1.5}.5 μl of each dilution is added to triplicate wells and the resulting AP content in each well is 5.50 ng, 1.74 ng, 0.55 ng, 0.18 ng. 100 μl of pNNP reagent must then be added to each of the standard wells. The plate must be incubated at 37° C. for 4 h. A volume of 50 μl of 3M NaOH is added to all wells. The results are quantified on a plate reader at 405 nm. The background subtraction option is used on blank wells filled with glycine buffer only. The template is set up to indicate the concentration of AP-conjugate in each standard well [5.50 ng; 1.74 ng; 0.55 ng; 0.18 ng]. Results are indicated as amount of bound AP-conjugate in each sample.

The studies described in this example tested activity in CTGF-4 protein. However, one skilled in the art could easily modify the exemplified studies to test the activity of CTGF-4 polynucleotides (e.g., gene therapy), agonists, and/or antagonists of CTGF-4.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference.

Further, the Sequence Listing submitted herewith, and the Sequence Listings submitted with U.S. application Ser. No. 09/325,019, filed on Jun. 3, 1999 (to which the present application claims priority under 35 U.S.C. §120) and U.S. Provisional Application No. 60/088,320, filed on Jun. 5, 1998 (to which the present application claims benefit under 35 U.S.C. §119(e)), in both computer and paper forms, are hereby incorporated by reference in entirety.

What is claimed is:

1. An isolated nucleic acid molecule comprising a polynucleotide having a nucleotide sequence at least 95% identical to a sequence selected from the group consisting of:
(a) a polynucleotide fragment of SEQ ID NO:1 or a polynucleotide fragment of the cDNA sequence included in ATCC Deposit No: 209816;
(b) a polynucleotide encoding a polypeptide fragment of SEQ ID NO:2 or the cDNA sequence included in ATCC Deposit No: 209816;
(c) a polynucleotide encoding an IGF-binding domain of SEQ ID NO:2 or the cDNA sequence included in ATCC Deposit No: 209816;
(d) a polynucleotide encoding a von Willebrand Factor Type C Repeat domain of SEQ ID NO:2 or the cDNA sequence included in ATCC Deposit No: 209816;
(e) a polynucleotide encoding a Sulfated Glycoconjugate Binding Motif domain of SEQ ID NO:2 or the cDNA sequence included in ATCC Deposit No: 209816;
(f) a polynucleotide encoding a C-terminal Dimerization and Receptor Binding domain of SEQ ID NO:2 or the cDNA sequence included in ATCC Deposit No: 209816;
(g) a polynucleotide encoding a polypeptide epitope of SEQ ID NO:2 or the cDNA sequence included in ATCC Deposit No: 209816;
(h) a polynucleotide encoding a polypeptide of SEQ ID NO:2 or the cDNA sequence included in ATCC Deposit No: 209816 having biological activity;
(i) a polynucleotide which is a variant of SEQ ID NO:1;
(j) a polynucleotide which is an allelic variant of SEQ ID NO:1;
(k) a polynucleotide which encodes a species homolog of the SEQ ID NO:2;
(l) a polynucleotide capable of hybridizing under stringent conditions to any one of the polynucleotides specified in (a)-(k), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding a mature form or a secreted protein.

3. The isolated nucleic acid molecule of claim 1, wherein the polynucleotide fragment comprises a nucleotide sequence encoding the sequence identified as SEQ ID NO:2 or the coding sequence included in ATCC Deposit No: 209816.

4. The isolated nucleic acid molecule of claim 1, wherein the polynucleotide fragment comprises the entire nucleotide sequence of SEQ ID NO:1 or the cDNA sequence included in ATCC Deposit No: 209816.

5. The isolated nucleic acid molecule of claim 2, wherein the nucleotide sequence comprises sequential nucleotide deletions from either the C-terminus or the N-terminus.

6. The isolated nucleic acid molecule of claim 3, wherein the nucleotide sequence comprises sequential nucleotide deletions from either the C-terminus or the N-terminus.

7. A recombinant vector comprising the isolated nucleic acid molecule of claim 1.

8. A method of making a recombinant host cell comprising the isolated nucleic acid molecule of claim 1.

9. A recombinant host cell produced by the method of claim 9.

10. The recombinant host cell of claim 9 comprising vector sequences.

11. An isolated polypeptide comprising an amino acid sequence at least 95% identical to a sequence selected from the group consisting of:
(a) a polypeptide fragment of SEQ ID NO:2 or the encoded sequence included in ATCC Deposit No: 209816;
(b) a polypeptide fragment of SEQ ID NO:2 or the encoded sequence included in ATCC Deposit No: 209816 having biological activity;
(c) a polypeptide domain of SEQ ID NO:2 or the encoded sequence included in ATCC Deposit No: 209816;
(d) a polypeptide epitope of SEQ ID NO:2 or the encoded sequence included in ATCC Deposit No: 209816;
(e) a mature form of a secreted protein;
(f) a full-length secreted protein;
(g) a variant of SEQ ID NO:2;
(h) an allelic variant of SEQ ID NO:2; or
(i) a species homologue of the SEQ ID NO:2.

12. The isolated polypeptide of claim 11, wherein the mature form or the full length secreted protein comprises sequential amino acid deletions from either the C-terminus or the N-terminus.

13. An isolated antibody that binds specifically to the isolated polypeptide of claim 11.

14. A recombinant host cell that expresses the isolated polypeptide of claim 11.

15. A method of making an isolated polypeptide comprising:
(a) culturing the recombinant host cell of claim 14 under conditions such that said polypeptide is expressed; and
(b) recovering said polypeptide.

16. The polypeptide produced by claim 15.

17. A method for preventing, treating, or ameliorating a medical condition which comprises administering to a mammalian subject a therapeutically effective amount of the polypeptide of claim 11.

18. A method of diagnosing a pathological condition or a susceptibility to a pathological condition in a subject related to expression or activity of a secreted protein comprising:
(a) determining the presence or absence of a mutation in the polynucleotide of claim 1;
(b) diagnosing a pathological condition or a susceptibility to a pathological condition based on the presence or absence of said mutation.

19. A method of diagnosing a, pathological condition or a susceptibility to a pathological condition in a subject related to expression or activity of a secreted protein comprising:
(a) determining the presence or amount of expression of the polypeptide of claim 11 in a biological sample;
(b) diagnosing a pathological condition or a susceptibility to a pathological condition based on the presence or amount of expression of the polypeptide.

20. A method for identifying binding partner to the polypeptide of claim 11 comprising:
(a) contacting the polypeptide of claim 11 with a binding partner; and
(b) determining whether the binding partner effects an activity of the polypeptide.

21. The gene corresponding to the cDNA sequence of SEQ ID NO:2.

22. A method of identifying an activity in a biological assay, wherein the method comprises:
(a) expressing SEQ ID NO:1 in a cell;
(b) isolating the supernatant;
(c) detecting an activity in a biological assay; and
(d) identifying the protein in the supernatant having the activity.

23. The product produced by the method of claim 22.

24. A method for preventing, treating, or ameliorating a medical condition which comprises administering to a mammalian subject a therapeutically effective amount of the polynucleotide of claim 1.


SEQUENCE LISTING (27 sequences)

SEQ ID NO: 1 - DNA, 3658 bp, Homo sapiens
  CDS (3)..(1007)
cggactttaccccagctccactggaggacacctcctcacgcccccaattctgcaagtggccatgtgagtgcccgccatccccaccccgctgcccgctgggggtcagcctcatcacagatggctgtgagtgctgtaagatgtgcgctcagcagcttggggacaactgcacggaggctgccatctgtgacccccaccggggcctctactgtgactacagcggggaccgcccgaggtacgcaataggagtgtgtgcacaggtggtcggtgtgggctgcgtcctggatggggtgcgctacaacaacggccagtccttccagcctaactgcaagtacaactgcacgtgcatcgacggcgcggtgggctgcacaccactgtgcctccgagtgcgccccccgcgtctctggtgcccccacccgcggcgcgtgagcatacctggccactgctgtgagcagtgggtatgtgaggacgacgccaagaggccacgcaagaccgcaccccgtgacacaggagccttcgatgctgtgggtgaggtggaggcatggcacaggaactgcatagcctacacaagcccctggagcccttgctccaccagctgcggcctgggggtctccactcggatctccaatgttaacgcccagtgctggcctgagcaagagagccgcctctgcaacttgcggccatgcgatgtggacatccatacactcattaaggcagggaagaagtgtctggctgtgtaccagccagaggcatccatgaacttcacacttgcgggctgcatcagcacacgctcctatcaacccaagtactgtggagtttgcatggacaataggtgctgcatcccctacaagtctaagactatcgacgtgtccttccagtgtcctgatgggcttggcttctcccgccaggtcctatggattaatgcctgcttctgtaacctgagctgtaggaatcccaatgacatctttgctgacttggaatcctaccctgacttctcagaaattgccaactaggcaggcacaaatcttgggtcttggggactaacccaatgcctgtgaagcagtcagcccttatggccaataacttttcaccaatgagccttagttaccctgatctggacccttggcctccatttctgtctctaaccattcaaatgacgcctgatggtgctgctcaggcccatgctatgagttttctccttgatatcattcagcatctactctaaagaaaaatgcctgtctctagctgttctggactacacccaagcctgatccagcctttccaagtcactagaagtcctgctggatcttgcctaaatcccaagaaatggaatcaggtagacttttaatatcactaatttcttctttagatgccaaaccacaagactctttgggtccattcagatgaatagatggaatttggaacaatagaataatctattatttggagcctgccaagaggtactgtaatgggtaattctgacgtcagcgcaccaaaactatcctgattccaaatatgtatgcacctcaaggtcatcaaacatttgccaagtgagttgaatagttgcttaattttgatttttaatggaaagttgtatccattaacctgggcattgttgaggttaagtttctcttcacccctacactgtgaagggtacagattaggtttgtcccagtcagaaataaaatttgataaacattcctgttgatgggaaaagcccccagttaatactccagagacagggaaaggtcagcccatttcagaaggaccaattgactctcacactgaatcagctgctgactggcagggctttgggcagttggccaggctcttccttgaatcttctcccttgtcctgcttggggttcataggaattggtaaggcctctggactggcctgtctggcccctgagagtggtgccctggaacactcctctactcttacagagccttgagagacccagctgcagaccatgccagacccactgaaatgaccaagacaggttcaggtaggggtgtgggtcaaaccaagaagtgggtgcccttggtagcagcctggggtgacctctagagctggaggctgtgggactccaggggcccccgtgttcaggacacatctattgcagagactcatttcacagcctttcgttctgctgaccaaatggccagttttctggtaggaagatggaggtttaccagttgtttagaaacagaaatagacttaataaaggtttaaagctgaagaggttgaagctaaaaggaaaaggttgttgttaatgaatatcaggctattatttattgtattaggaaaatataatatttactgttagaattcttttatttagggccttttctgtgccagacattgctctcagtgctttgcatgtattagctcactgaatcttcacgacaatgttgagaagttcccattattatttctgttcttacaaatgtgaaacggaagctcatagaggtgagaaaactcaaccagagtcacccagttggtgactgggaaagttaggattcagatcgaaattggactgtctttataacccatattttccccctgtttttagagcttccaaatgtgtcagaataggaaaacattgcaataaatggcttgattttttaatgtcatttttccctcttatagtctttctagctccttttcaaaagacgagaatatctgattttctgataatttaggtgcttaagcatccaaaatacatgggacacacaaaaatccaggaatcccctgtagcttattccctctttcccatcggaaccagctctcatcacacatttaaaagatgattctgtttacccaatgctgcatattgaatgttgtgtagttattcacagggaattctgtgcagtgtgcagagagattcctaaacgggaaaaggactgggaatacatcctccttactgtgacctccccaaaacctagtccagtgcaaggtatacagtggtgctcattaaatacttgatgaatacaggaagctgtgcatgtgttcctacttttattcgaagctctcttcttccaaagctacatgaaaatagaattttaacagtcaaaattttatattaagtgtcttagcaaaagagacatttaatatttcaaagaaatgcatatgtatgtatacatatatttgtgtatgcgtatgcaagaattcttgtataaagagaattcactccatgaatgatctcttctgtaagtcagtgtgaatcatgttagattttctgagagtgaaaacacctgccatctacaaattacaaggctggataacagctcactccatttgaaattcagtggaaacccaagagctaggttcttactgaatttgcatctcaatttgggaaactgaacttagctttcaaagatcataggaagtctggttggagaaactagggattattctggcaatgggtggaggaaggtggtcagaataacccagtcgccattggttttgagaaacggaactatcttatgcagagcccggagggcaagtctcaaacccatgggttgaagccatggagaaggaaatttggatccaatgtaatgaagctctttctaagtcagaatttccctgcaatggtgtggcctgattcaataaaaattaagaataataaatataatggaaaaaaaaaaaaaaaaaaaaa

SEQ ID NO: 2 - protein, 335 aa, Homo sapiens
DFTPAPLEDTSSRPQFCKWPCECPPSPPRCPLGVSLITDGCECCKMCAQQLGDNCTEAAICDPHRGLYCDYSGDRPRYAIGVCAQVVGVGCVLDGVRYNNGQSFQPNCKYNCTCIDGAVGCTPLCLRVRPPRLWCPHPRRVSIPGHCCEQWVCEDDAKRPRKTAPRDTGAFDAVGEVEAWHRNCIAYTSPWSPCSTSCGLGVSTRISNVNAQCWPEQESRLCNLRPCDVDIHTLIKAGKKCLAVYQPEASMNFTLAGCISTRSYQPKYCGVCMDNRCCIPYKSKTIDVSFQCPDGLGFSRQVLWINACFCNLSCRNPNDIFADLESYPDFSEIAN

SEQ ID NO: 3 - protein, 367 aa, Mus musculus
MRWLLPWTLAAVAVLRVGNILATALSPTPTTMTFTPAPLEETTTRPEFCKWPCECPQSPPRCPLGVSLITDGCECCKICAQQLGDNCTEAAICDPHRGLYCDYSGDRPRYAIGVCAQVVGVGCVLDGVRYTNGESFQPNCRYNCTCIDGTVGCTPLCLSPRPPRLWCRQPRHVRVPGQCCEQWVCDDDARRPRQTALLDTRAFAASGAVEQRYENCIAYTSPWSPCSTTCGLGISTRISNVNARCWPEQESRLCNLRPCDVDIQLHIKAGKKCLAVYQPEEATNFTLAGCVSTRTYRPKYCGVCTDNRCCIPYKSKTISVDFQCPEGPGFSRQVLWINACFCNLSCRNPNDIFADLESYPDFEEIAN

SEQ ID NO: 4 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 5 - protein, 381 aa, Homo sapiens [sequence omitted]

SEQ ID NO: 6 - protein, 357 aa, Homo sapiens
MQSVQSTSFCLRKQCLCLTFLLLHLLGQVAATQRCPPQCPGRCPATPPTCAPGVRAVLDGCSCCLVCARQRGESCSDLEPCDESSGLYCDRSADPSNQTGICTAVEGDNCVFDGVIYRSGEKFQPSCKFQCTCRDGQIGCVPRCQLDVLLPEPNCPAPRKVEVPGECCEKWICGPDEEDSLGGLTLAAYRPEATLGVEVSDSSVNCIEQTTEWTACSKSCGMGFSTRVTNRNRQCEMLKQTRLCMVRPCEQEPEQPTDKKGKKCLRTKKSLKAIHLQFKNCTSLHTYKPRFCGVCSDGRCCTPHNTKTIQAEFQCSPGQIVKKPVMVIGTCTCHTNCPKNNEAFLQELELKTTRGKM

SEQ ID NO: 7 - DNA, 540 bp, Homo sapiens
  misc_feature (162): n equals a, t, g or c
ggcacagttttttttttccattatatttattattcttaatttttattgaatcaggccacaccattgcagggaaattctgacttagaaagagcttcattacattggatccaaatttccttctccatggcttcaacccatgggtctgagacttgccctccggcnctgcataagatagttccgtttctcaaaaccaatggcgactgggttattctgaccaccttcctccacccattgccagaataatccctagtttctccaaccagacttcctatgatctttgaaagctaattcagtttcccaaattggagatgcaattcnagtaagaacctagccctngggtttccanggaattccaatgggagtgagccgttatccagcctgnaaattngtaaanggcaggnggtttccanccccagaaaatncaacaggattcacacggcttacggaggggtcctccaggggggattcccttaaaacagaatctggcaaaggnncncaantttggaacaacaagcatcctggaaataaagncctggcagg

SEQ ID NO: 8 - DNA, 539 bp, Homo sapiens
  misc_feature (5): n equals a, t, g or c
ggcanagttttttttttccattatatttattattcttaatttttattgaatcaggccacaccattgcagggaaattctgacttagaaagagcttcattacattggatccaaatttccttctccatggcttcaacccatgggtctgagacttgccctccggntnctgcataagatagttccgtttctcaaaaccaatggcgactgggttattctgaccaccttcctccacccattgccagaataatccctagtttctccaaccagacttcctatgatctttgaaagctaagttcatttcccaattgagatgcaattccagtaagaaccaagccttggggttnccanggatttcaatgggntgngcgttnnccagcntgnaattggnaaaggcaggggtttncacccccggaaaaccaaaagggttccaccggcttnacgaggggccntccagggggaatncctttaanaagatctggaagggancccanttngtnaaaaaagncttctggaaaaaagccttgcaggctaaaaatgggggg

SEQ ID NO: 9 - DNA, 311 bp, Homo sapiens
  misc_feature (177): n equals a, t, g or c
attgaatgttgtgtagttattcacagggaattctgtgcagtgtgcagagagattcctaaacgggaaaaggactgggaatacatcctccttactgtgacctccccaaaacctagtccagtgcaaggtatacagtggtgctcattaaatacttgatgaatacaggaagctgtgcatgtnttcctacttttnttcgaagctctcttcttccaaagctacatgaaaatagaattttaacagtcaaaattttatattaagtgccttagcaaaagagacatttaatattttcaaagaaatgcatatgtatgtataca

SEQ ID NO: 10 - DNA, 197 bp, Homo sapiens
ctcttctgtaagtcagtgtgaatcatgttagattttctgagagtgaaaacacctgccatctacaaattacaaggctggataacagctcactccatttgaaattcagtggaaacccaagagctaggttcttactggaatttgcatctcaatttgggaaactgaacttagctttcaaagatcataggaagtcttgttgg

SEQ ID NO: 11 - DNA, 484 bp, Homo sapiens
  misc_feature (39): n equals a, t, g or c
ggcagagtgtatacatatatttgtgtatgcgtatgaagnaattcttgtataaagagaattcactccatgaatganctcttctgtagtnnagtgtgaatcatgtnagatttnctaagagtgaaaaacacctgccatctacaaattnacaaggctggataacagctncactnccatttgaaaattcagtgggaaacccaagagctaggttcttactggaatttgccatctncaatttgggnaaactgaaacttaggctttccaaaggttcatagggaagtctgggttggaggaaactaggggattattcctgggcaatggggtgggaggnaggtgggtncagaattaaccccgttcgncctttggttttgaggaacggnantatcttatggcgngcccnggggaagttcttcggaccctngggttnnaggcctgggggagggaattttgggtcccatgtatgaggtctttctaggtcnggatttcc

SEQ ID NO: 12 - DNA, 236 bp, Homo sapiens
  misc_feature (38): n equals a, t, g or c
ggcacagcgctcagcagcttggggacaactgcacggangctgccatctgtgacccccaccggccgctgctactgtgactacatcggggacccacgaggtacgcaatagggagtgtgtgcacaggtggtcggtgtgggctgcgtcctgggatggggtgcgctacaacaacggaccagtccttnccagcctaaactggcaatgacaactgccacgtgncatncggacggnnacggtgg

SEQ ID NO: 13 - DNA, 32 bp, Homo sapiens
cgcggatccgcgatggactttaccccagctcc

SEQ ID NO: 14 - DNA, 39 bp, Homo sapiens
ctagtctagactaggttggcaatttctgagaagtcaggg

SEQ ID NO: 15 - DNA, 30 bp, Homo sapiens
cgcggatccgcgcgactttaccccagctcc

SEQ ID NO: 16 - DNA, 39 bp, Homo sapiens
ctagggtaccctaggttggcaatttctgagaagtcaggg

SEQ ID NO: 17 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 18 - protein, 5 aa, Homo sapiens
  SITE (3): Xaa equals any amino acid
WSXWS

SEQ ID NO: 19 - DNA, 86 bp, Homo sapiens
gcgcctcgagatttccccgaaatctagatttccccgaaatgatttccccgaaatgatttccccgaaatatctgccatctcaattag

SEQ ID NO: 20 - DNA, 27 bp, Homo sapiens
gcggcaagctttttgcaaagcctaggc

SEQ ID NO: 21 - DNA, 271 bp, Homo sapiens
ctcgagatttccccgaaatctagatttccccgaaatgatttccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 22 - DNA, 32 bp, Homo sapiens
gcgctcgagggatgacagcgatagaaccccgg

SEQ ID NO: 23 - DNA, 31 bp, Homo sapiens
gcgaagcttcgcgactccccggatccgcctc

SEQ ID NO: 24 - DNA, 12 bp, Homo sapiens
ggggactttccc

SEQ ID NO: 25 - DNA, 73 bp, Homo sapiens
gcggcctcgaggggactttcccggggactttccggggactttccgggactttccatcctgccatctcaattag

SEQ ID NO: 26 - DNA, 27 bp, Homo sapiens
gcggcaagctttttgcaaagcctaggc

SEQ ID NO: 27 - DNA, 256 bp, Homo sapiens
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt